(19) United States
(12) Patent Application Publication
(10) Pub. No.: US 2013/0210809 A1
(43) Pub. Date: Aug. 15, 2013

(54) NOVEL 2-AMINO-4-PYRAZOLYL-THIAZOLE DERIVATIVES AND THEIR USE AS ALLOSTERIC MODULATORS OF METABOTROPIC GLUTAMATE RECEPTORS

(71) Applicants: Christelle Boléa, Geneva (CH); Sylvain Celanire, Geneva (CH); Cédric Boudou, Geneva (CH); Lam Tang, Geneva (CH); Jean-Philippe Rocher, Geneva (CH); Nigel John Liverton, Harleysville, PA (US)

(72) Inventors: Christelle Boléa, Geneva (CH); Sylvain Celanire, Geneva (CH); Cédric Boudou, Geneva (CH); Lam Tang, Geneva (CH); Jean-Philippe Rocher, Geneva (CH); Nigel John Liverton, Harleysville, PA (US)

(21) Appl. No.: 13/808,899

(22) PCT Filed: Jul. 12, 2011
(86) PCT No.: PCT/US11/01222

§ 371 Date: Mar. 25, 2013

Related U.S. Application Data
(60) Provisional application No. 61/399,564, filed on Jul. 14, 2010.

(51) Int. Cl. C07D 417/14; A61K 45/06; A61K 31/506; A61K 31/53; A61K 31/5377; A61K 31/4439; A61K 31/433; C07D 487/04; A61K 31/541

(52) U.S. Cl. CPC C07D 417/14; C07D 487/04; A61K 45/06; A61K 31/506; A61K 31/541; A61K 31/5377; A61K 31/4439; A61K 31/433; A61K 31/53
USPC 514/217; 544/331; 544/58.2; 544/122; 544/124; 546/270.7; 548/128; 544/182; 514/275; 514/227.8; 514/236.5; 514/341; 514/361; 514/242

(57) ABSTRACT

The present invention relates to novel compounds of Formula (1), wherein M, A and B are defined as in Formula (I); invention compounds are modulators of metabotropic glutamate receptors-subtype 4 (“mGluR4”) which are useful for the treatment or prevention of central nervous system disorders as well other disorders modulated by mGluR4 receptors. The invention is also directed to pharmaceutical compositions and the use of such compounds in the manufacture of medicaments, as well as to the use of such compounds for the prevention and treatment of such diseases in which mGluR4 is involved.

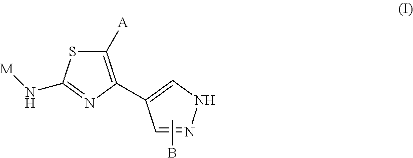

### SUMMARY OF THE INVENTION

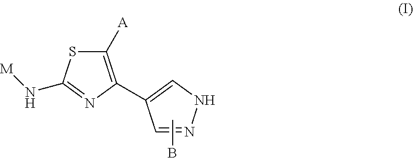

The present invention relates to novel compounds of Formula (I), wherein M, A and B are defined as in Formula (I); invention compounds are modulators of metabotropic glutamate receptors-subtype 4 (“mGluR4”) which are useful for the treatment or prevention of central nervous system disorders as well as other disorders modulated by mGluR4 receptors. The invention is also directed to pharmaceutical compositions and the use of such compounds in the manufacture of medicaments, as well as to the use of such compounds for the prevention and treatment of such diseases in which mGluR4 is involved.

### BACKGROUND OF THE INVENTION

Glutamate is the major amino-acid transmitter in the mammalian central nervous system (CNS). Glutamate plays a major role in numerous physiological functions, such as learning and memory but also sensory perception, development of synaptic plasticity, motor control, respiration and regulation of cardiovascular function. Furthermore, glutamate is at the center of several different neurological and psychiatric diseases, where there is an imbalance in glutamatergic neurotransmission.

Glutamate mediates synaptic neurotransmission through the activation of ionotropic glutamate receptor channels (iGluRs), namely the NMDA, AMPA and kainate receptors which are responsible for fast excitatory transmission (Nakanishi et al., (1998) Brain Res. Rev., 26:230-235).

In addition, glutamate activates metabotropic glutamate receptors (mGluRs) which have a more modulatory role that contributes to the fine-tuning of synaptic efficacy.

The mGluRs are G protein-coupled receptors (GPCRs) with seven-transmembrane spanning domains and belong to GPCR family 3 along with the calcium-sensing, GABAb and pheromone receptors.

The mGluR family is composed of eight members. They are classified into three groups (group I comprising mGluR1 and mGluR5; group II comprising mGluR2 and mGluR3; group III comprising mGluR4, mGluR6, mGluR7 and mGluR8) according to sequence homology, pharmacological profile and nature of intracellular signalling cascades activated (Schoepp et al., (1999) Neuropharmacology, 38:1431-1476).

Glutamate activates the mGluRs through binding to the large extracellular amino-terminal domain of the receptor, herein called the orthosteric binding site. This activation induces a conformational change of the receptor which results in the activation of the G-protein and intracellular signalling pathways.

In the central nervous system, mGluR4 receptors are expressed most intensely in the cerebellar cortex, basal ganglia, sensory relay nuclei of the thalamus and hippocampus (Bradley et al., (1999) Journal of Comparative Neurology, 407:33-46; Corti et al., (2002) Neuroscience, 110:403-420). The mGluR4 subtype is negatively coupled to adenylate cyclase via activation of the Gαi/o protein, is expressed primarily on presynaptic terminals, functioning as an autoreceptor or heteroceptor and activation of mGluR4 leads to decreases in transmitter release from presynaptic terminals (Corti et al., (2002) Neuroscience, 110:403-420; Millan et al., (2002) Journal of Biological Chemistry, 277:47796-47803; Valenti et al., (2003) Journal of Neuroscience, 23:7218-7226).

Orthosteric agonists of mGluR4 are not selective and activate the other Group III mGluRs (Schoepp et al., (1999) Neuropharmacology, 38:1431-1476). The Group III orthosteric agonist L-AP4 (L-2-amino-4-phosphonobutyrate) was able to reduce motor deficits in animal models of Parkinson's disease (Valenti et al., (2003) J. Neurosci., 23:7218-7226) and decrease excitotoxicity (Bruno et al., (2000) J. Neurosci., 20; 6413-6420) and these effects appear to be mediated through mGluR4 (Marino et al., (2005) Curr. Topics Med. Chem., 5:885-895). In addition to L-AP4, ACPT-1, another selective group III mGluR agonist has been shown to caused a dose and structure-dependent decrease in haloperidol-induced catalepsy and attenuated haloperidol-increased Proenkephalin mRNA expression in the striatum (Konieczny et al., (2007) Neuroscience, 145:611-620). Furthemore, Lopez et al. (2007, J. Neuroscience, 27:6701-6711) have shown that bilateral infusions of ACPT-I or L-AP4 into the globus pallidus fully reversed the severe akinetic deficits produced by 6-hydroxydopamine lesions of nigrostriatal dopamine neurons in a reaction-time task without affecting the performance of controls. In addition, the reversal of haloperidol-induced catalepsy by intrapallidal ACPT-1 was prevented by concomitant administration of a selective group III receptor antagonist (RS)-alpha-cyclopropyl-4-phosphonophenylglycine. The opposite effects produced by group III mGluR activation in the SNr strongly suggest a role of mGluR4 rather than others mGluR receptor sub-types in normalizing basal ganglia activity (Lopez et al. 2007).

These results suggest that, among mGluR subtypes, mGluR4 is believed to be the most interesting novel drug target for the treatment of Parkinson's disease (for a review see Conn et al., (2005) Nature Review Neuroscience, 6:787-798).

Symptoms of Parkinson's disease appear to be due to an imbalance in the direct and indirect output pathways of the basal ganglia, and reduction of transmission at the inhibitory GABAergic striato-pallidal synapse in the indirect pathway may result in alleviation of these symptoms (Marino et al., (2002) Amino Acids, 23:185-191).

mGluR4 is more abundant in striato-pallidal synapses than in striato-nigral synapses, and its localization suggests function as a presynaptic heteroreceptor on GABAergic neurons (Bradley et al., (1999) Journal of Comparative Neurology, 407:33-46) suggesting that selective activation or positive modulation of mGluR4 would decrease GABA release in this synapse thereby decreasing output of the indirect pathway and reducing or eliminating the Parkinson's disease symptoms. Classical treatment of Parkinsonism typically involves the use of levodopa combined with carbidopa (SINEMET™) or benserazide (MADOPAR™). Dopamine agonists such as bromocriptine (PARLODEL™), lisuride and pergolide (CELANCE™) act directly on dopamine receptors and are also used for the treatment of Parkinsonism. These molecules have the same side-effect profile as levodopa.

A new avenue for developing selective compounds acting at mGluRs is to identify molecules that act through allosteric mechanisms, modulating the receptor by binding to a site different from the highly conserved orthosteric binding site.

Positive allosteric modulators of mGluRs have emerged recently as novel pharmacological entities offering this attractive alternative. This type of molecule has been discovered for mGluR1, mGluR2, mGluR4, mGluR5, mGluR7 and mGluR8 (Knoflach F. et al. (2001) Proc. Natl. Acad. Sci. USA, 98:13402-13407; Johnson M. P. et al., (2002) Neuropharmacology, 43:799-808; O'Brien J. A. et al., (2003) Mol. Pharmacol., 64:731-740; Johnson M. P. et al., (2003) J. Med. Chem., 46:3189-3192; Marino M. J. et al., (2003) Proc. Natl. Acad. Sci. USA, 100:13668-13673; Mitsukawa K. et al., (2005) Proc. Natl. Acad. Sci. USA, 102(51):18712-18717; Wilson J. et al., (2005) Neuropharmacology, 49:278; for a review see Mutel V., (2002) Expert Opin. Ther. Patents, 12:1-8; Kew J. N., (2004) Pharmacol. Ther., 104(3):233-244; Johnson M. P. et al., (2004) Biochem. Soc. Trans., 32:881-887; recently Ritzen A., Mathiesen, J. M. and Thomsen C., (2005) Basic Clin. Pharmacol. Toxicol., 97:202-213).

In particular molecules have been described as mGluR4 positive allosteric modulators (Maj et al., (2003) Neuropharmacology, 45:895-906; Mathiesen et al., (2003) British Journal of Pharmacology, 138:1026-1030). It has been demonstrated that such molecules have been characterized in in vitro systems as well as in rat brain slices where they potentiated the effect of L-AP4 in inhibiting transmission at the striatopallidal synapse. These compounds do not activate the receptor by themselves (Marino et al., (2003) Proc. Nat. Acad. Sci. USA, 100:13668-13673). Rather, they enable the receptor to produce a maximal response to a concentration of glutamate or the Group III orthosteric agonist L-AP4 which by itself induces a minimal response.

PHCCC (N-phenyl-7-(hydroxyimino)cyclopropa[b]chromen-1a-carboxamide), a positive allosteric modulator of mGluR4 not active on other mGluRs (Maj et al., (2003) Neuropharmacology, 45:895-906), has been shown to be efficacious in animal models of Parkinson's disease thus representing a potential novel therapeutic approach for Parkinson's disease as well as for other motor disorders and disturbances (Marino et al., (2003) Proc. Nat. Acad. Sci. USA, 100:13668-13673), neurodegeneration in Parkinson's disease (Marino et al., (2005) Curr. Topics Med. Chem., 5:885-895; Valenti et al., (2005) J. Pharmacol. Exp. Ther., 313:1296-1304; Vernon et al., (2005) Eur. J. Neurosci., 22:1799-1806, Battaglia et al., (2006) J. Neurosci., 26:7222-7229), and neurodegeneration in Alzheimer's disease or due to ischemic or traumatic insult (Maj et al., (2003) Neuropharmacology, 45:895-906).

PHCCC also has been shown to be active in an animal model of anxiety (Stachowicz et al., (2004) Eur. J. Pharmacol., 498:153-156). Previously, ACPT-1 has been shown to produce a dose-dependent anti-conflict effect after intrahippocampal administration and anti-depressant-like effects in rats after intracerebroventricular administration (Tatarczynska et al., (2002) Pol. J. Pharmacol., 54(6):707-710). More recently, ACPT-1 has also been shown to have anxiolytic-like effects in the stress-induced hyperthermia, in the elevated-plus maze in mice and in the Vogel conflict test in rats when injected intraperitoneally (Stachowicz et al., (2009) Neuropharmacology, 57(3): 227-234).

Activation of mGluR4 receptors which are expressed in α- and F-cells in the islets of Langerhans inhibits glucagon secretion. Molecules which activate or potentiate the agonist activity of these receptors may be an effective treatment for hyperglycemia, one of the symptoms of type 2 diabetes (Uehara et al., (2004) Diabetes, 53:998-1006).

The β-chemokine RANTES is importantly involved in neuronal inflammation and has been implicated in the pathophysiology of multiple sclerosis. Activation of Group III mGluRs with L-AP4 reduced the synthesis and release of RANTES in wild-type cultured astrocytes, whereas the ability of L-AP4 to inhibit RANTES was greatly decreased in astrocyte cultures from mGluR4 knockout mice (Besong et al., (2002) Journal of Neuroscience, 22:5403-5411). These data suggest that positive allosteric modulators of mGluR4 may be an effective treatment for neuroinflammatory disorders of the central nervous system, including multiple sclerosis and related disorders.

Two different variants of the mGluR4 receptor are expressed in taste tissues and may function as receptors for the umami taste sensation (Monastyrskaia et al., (1999) Br. J. Pharmacol., 128:1027-1034; Toyono et al., (2002) Arch. Histol. Cytol., 65:91-96).

Thus positive allosteric modulators of mGluR4 may be useful as taste agents, flavour agents, flavour enhancing agents or food additives.

There is anatomical evidence that the majority of vagal afferents innervating gastric muscle express group III mGluRs (mGluR4, mGluR6, mGluR7 and mGluR8) and actively transport receptors to their peripheral endings (Page et al., (2005) Gastroenterology, 128:402-10). Recently, it was shown that the activation of peripheral group III mGluRs inhibited vagal afferents mechanosensitivity in vitro which translates into reduced triggering of transient lower esophageal sphincter relaxations and gastroesophageal reflux in vivo (Young et al., (2008) Neuropharmacol, 54:965-975). Labelling for mGluR4 and mGluR8 was abundant in gastric vagal afferents in the nodose ganglion, at their termination sites in the nucleus tractus solitarius and in gastric vagal motoneurons. These data suggest that positive allosteric modulators of mGluR4 may be an effective treatment for gastroesophageal reflux disease (GERD) and lower esophageal disorders and gastro-intestinal disorders.

International patent publication WO2005/007096 has described mGluR4 receptor positive allosteric modulator useful, alone or in combination with a neuroleptic agent, for treating or preventing movement disorders. However, none of the specifically disclosed compounds are structurally related to the compounds of the invention. Recently, new mGluR4 receptor positive allosteric modulators have been described: pyrazolo[3,4-d]pyrimidine derivatives (Niswender et al., (2008) Bioorganic & Medicinal Chemistry Letters, 18(20):5626-5630), functionalised benzylidene hydrazinyl-3-methylquinazoline and bis-2,3-dihydroquinazolin-4(1H)-one (Williams et al., (2009) Bioorganic & Medicinal Chemistry Letters, 19:962-966) and heterobiarylamides (Engers et al, (2009) Journal of Medicinal Chemistry, 52 (14): 4115-4118). Niswender et al., has described (±)-cis-2-(3,5-dichlorophenylcarbamoyl)cyclohexane carboxylic acid (2008) Molecular Pharmacology, 74(5):1345-1358), as a positive allosteric modulator of mGluR4 also having agonist activity. This moderately active molecule has demonstrated evidence of efficacy following icy injection in rat models of Parkinson's disease. International patent publications WO2009/010454 and WO2009/010455 have mentioned amido derivatives and novel heteroaromatic derivatives, respectively, as positive allosteric modulators of metabotropic glutamate receptors. The subject of the latter case has been examined in the following article East Stephen P. et al., (2010) Expert Opin. Ther. Patents, 20(3) 441-445. Williams R. et al., has described in (2010) ACS Chemical Neuroscience, 1(6): 411-419, the “Re-exploration of the PHCCC scaffold”.

International patent publications WO2010/079239 and WO2011/010222 have described new amino-thiazole derivatives and new ether-thiazole derivatives, respectively.

International patent publication WO2010/079238 has described novel tricyclic heteroaromatic derivatives and their use as positive allosteric modulators of mGluRs. More recently, a review on new progress on the identification of metabotropic glutamate 4 receptor ligands and their potential utility as CNS therapeutics (Robichaud A. et al., (14 Jun. 2011) ACS Chemical Neuroscience, DOI: 10.1021/cn200043e, http://pubs.acs.org) has cited some of the examples described in the WO2010/079238 patent application; Hong S.-P et al, (20 Jun. 2011) J. Med. Chem., DOI: 10.1021/jm200290z, http://pubs.acs.org) have described tricyclic thiazolopyrazole derivatives as metabotropic glutamate receptor 4 positive allosteric modulators.

International patent publication WO2004/110350 describes a class of aminothiazole compounds as modulators of amyloid-beta (Aβ) levels. Synthesis of 4-pyrazolylthiazoles and 4-pyrazolylmercaptoimidazoles using [hydroxyl(tosyloxy)iodo]benzene has been described by Singh et al. in (1997) Journal of the Indian Chemical Society, 74(11-12): 940-942.

The present inventors have discovered novel aminothiazole compounds of general Formula (I) which, surprisingly, show potent activity and selectivity on the mGluR4 receptor. The compounds of the invention demonstrate advantageous properties over compounds of the prior art. Improvements have been observed in one or more of the following characteristics of the compounds of the invention: the potency on the target, the selectivity for the target, the bioavailability, the brain penetration, and the activity in behavioural models.

Such aminothiazole derivatives are useful for treating or preventing a condition in a mammal, including a human, the treatment or prevention of which is affected or facilitated by the neuromodulatory effect of mGluR4 modulators. In the case of the treatment of movement disorders such as Parkinson's disease, the compounds of the invention can be used alone or in combination with an agent selected from the group consisting of: levodopa, levodopa with a selective extracerebral decarboxylase inhibitor, carbidopa, entacapone, a COMT inhibitor, a dopamine agonist, an anticholinergic, a cholinergic agonist, a butyrophenone neuroleptic agent, a diphenylbutylpiperidine neuroleptic agent, a heterocyclic dibenzazepine neuroleptic agent, an indolone neuroleptic agent, a phenothiazine neuroleptic agent, a thioxanthene neuroleptic agent, an NMDA receptor antagonist, an MAO-B inhibitor, an mGluR5 antagonist or an A_{2A} antagonist.

### DETAILED DESCRIPTION OF THE INVENTION

The invention relates to compounds having metabotropic glutamate receptor 4 modulator activity. In its most general compound aspect, the present invention provides a compound according to Formula (I),

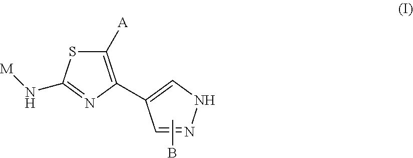

a pharmaceutically acceptable acid or base addition salt thereof, a stereochemically isomeric form thereof and an N-oxide form thereof, wherein:
A radical is selected from the group of hydrogen, halogen, —CN, —CF₃ and an optionally substituted radical selected from the group of —(C₁-C₆)alkyl, —(C₁-C₆)haloalkyl, —(C₃-C₂)cycloalkyl, —(C₁-C₆)cyanoalkyl, —(C₁-C₆)alkylene-heteroaryl, —(C₁-C₆)alkylene-heterocycle, —(C₁-C₆)alkylene-aryl, aryl, heteroaryl, heterocycle, —(C₀-C₆)alkyl-OR¹, —O—(C₂-C₆)alkylene-OR¹, —NR¹(C₂-C₆)alkylene-OR², —(C₃-C₂)cycloalkyl-(C₁-C₆)alkyl, —O—(C₃-C₂)cycloalkyl-(C₁-C₆)alkyl, —NR¹—(C₃-C₇)cycloalkyl-(C₁-C₆)alkyl, —(C₁-C₆)haloalkylene-OR¹, —(C₁-C₆)haloalkylene-NR¹R², —(C₀-C₆)alkyl-S—R′, —O—(C₂-C₆)alkyl ene-S—R¹, —NR¹—(C₂-C₆)alkylene-S—R², —(C₀-C₆)alkyl-S(═O)—R¹, —O—(C₁-C₆)alkylene-S(═O)—R¹, —NR¹—(C₁-C₆)alkylene-S(═O)—R², —(C₀-C₆)alkyl-S(═O)₂—R¹, —O—(C₁-C₆)alkylene-S(═O)₂—R¹, —NR¹—(C₁-C₆)alkylene-S(═O)₂—R², —(C₀-C₆)alkyl-NR¹R², —O—(C₂-C₆)alkylene-NR¹R², —NR¹—(C₂-C₆)alkylene-NR²R³, —(C₀-C₆)alkyl-S(═O)₂NR¹R², —O—(C₁-C₆)alkylene-S(═O)₂NR¹R², —NR¹—(C₁-C₆)alkylene-S(═O)₂NR²R³, —(C₀-C₆)alkyl-NR¹—S(═O)₂R², —O—(C₂-C₆)alkylene-NR¹—S(═O)₂R², —NR¹—(C₂-C₆)alkylene-NR²—S(═O)₂R³, —(C₀-C₆)alkyl-C(═O)—NR¹R², —O—(C₁-C₆)alkylene-C(═O)—NR¹R², —NR¹—(C₁-C₆)alkylene-C(═O)—NR²R³, —(C₀-C₆)alkyl-NR C(═O)—R², —O—(C₂-C₆)alkylene-NR¹C(═O)—R², —NR¹—(C₂-C₆)alkylene-NR²C(═O)—R³, —(C₀-C₆)alkyl-C(═O)—R¹, —O—(C₁-C₆)alkylene-C(═O)—R¹ and —NR¹—(C₁-C₆)alkylene-C(═O)—R²;
R¹, R² and R³ are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₄-C₁₀)alkylene-cycloalkyl, heteroaryl, —(C₁-C₆)alkylene-heteroaryl, aryl, heterocycle, —(C₁-C₆)alkylene-heterocycle and —(C₁-C₆)alkylene-aryl;
Any two radicals of R (R¹, R² or R³) may be taken together to form an optionally substituted 3 to 10 membered carbocyclic or heterocyclic ring;
B radical is selected from the group of hydrogen, halogen, —CN, —OH, —CF₃, —SH, —NH₂ and an optionally substituted radical selected from the group of —(C₁-C₆)alkyl, —(C₁-C₆)haloalkyl, —(C₃-C₇)cycloalkyl, —(C₁-C₆)cyanoalkyl, —(C₁-C₆)alkylene-heteroaryl, —(C₁-C₆)alkylene-heterocycle, —(C₁-C₆)alkylene-aryl, aryl, heteroaryl, heterocycle, —(C₀-C₆)allyl-OR⁴, —O—(C₂-C₆)allylene-OR⁴, —NR⁴(C₂-C₆)alkylene-OR⁵, —(C₃-C₇)cycloalkyl-(C₁-C₆)alkyl, —O—(C₃-C₇)cycloalkyl-(C₁-C₆)alkyl, —NR⁴—(C₃-C₇)cycloalkyl-(C₁-C₆)alkyl, —(C₁-C₆)haloalkylene-OR⁴, —(C₁-C₆)haloalkylene-NR⁴R⁵, —(C₀-C₆)alkyl-S—R⁴, —O—(C₂-C₆)alkylene-S—R⁴, —NR⁴—(C₂-C₆)alkylene-S—R⁵, —(C₀-C₆)alkyl-S(═O)—R⁴, —O—(C₁-C₆)allylene-S(═O)—R⁴, —NR⁴—(C₁-C₆)alkylene-S(═O)—R⁵, —(C₀-C₆)alkyl-S(═O)₂—R⁴, —O—(C₁-C₆)alkylene-S(═O)₂—R⁴, —NR⁴—(C₁-C₆)alkylene-S(═O)₂—R⁵, —(C₀-C₆)alkyl-NR⁴R⁵, —O—(C₂-C₆)alkylene-NR⁴R⁵, —NR⁴—(C₂-C₆)allylene-NR⁵R⁶, —(C₀-C₆)alkyl-S(═O)₂NR⁴R⁵, —O—(C₁-C₆)alkylene-S(═O)₂NR⁴R⁵, —NR⁴—(C₁-C₆)alkylene-S(═O)₂NR⁵R⁶, —(C₀-C₆)alkyl-NR⁴—S(═O)₂R⁵, —O—(C₂-C₆)alkylene-NR⁴—S(═O)₂R⁵, —NR⁴—(C₂-C₆)alkylene-NR⁵—S(═O)₂R⁶, —(C₀-C₆)alkyl-C(═O)—NR⁴R⁵, —O—(C₁-C₆)alkylene-C(═O)—NR⁴R⁵, —NR⁴—(C₁-C₆)alkylene-C(═O)—NR⁵R⁶, —(C₀-C₆)alkyl-NR⁴C(═O)—R⁵, —O—(C₂-C₆)alkylene-NR⁴C(═O)—R⁵, —NR⁴—(C₂-C₆)alkylene-NR⁵C(═O)—R⁶, —(C₀-C₆)alkyl-C(═O)—R⁴, —O—(C₁-C₆)alkylene-C(═O)—R⁴ and NR⁴—(C₁-C₆)allylene-C(═O)—R⁵;
R⁴, R⁵ and R⁶ are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₄-C₁₀)alkylene-cycloalkyl, heteroaryl, —(C₁-C₆)alkylene-heteroaryl, aryl, heterocycle, —(C₁-C₆)alkylene-heterocycle and —(C₁-C₆)alkylene-aryl;
Any two radicals of R (R⁴, R⁵ or R⁶) may be taken together to form an optionally substituted 3 to 10 membered carbocyclic or heterocyclic ring;
M is an optionally substituted heteroaryl;
provided that according to proviso (i) the compound is not:
4-(3-Methyl-1H-pyrazol-4-yl)-N-(pyridin-2-yl)thiazol-2-amine
N-(Pyridin-2-yl)-4-(3-(trifluoromethyl)-1H-pyrazol-4-yl)thiazol-2-amine
4-(3-Isopropyl-1H-pyrazol-4-yl)-N-(pyridin-2-yl)thiazol-2-amine
5-Methyl-4-(3-methyl-1H-pyrazol-4-yl)-N-(pyridin-2-yl)thiazol-2-amine
5-Methyl-4-(1H-pyrazol-4-yl)-N-(pyridin-2-yl)thiazol-2-amine
5-Methyl-N-(6-methylpyridin-2-yl)-4-(1H-pyrazol-4-yl)thiazol-2-amine
5-Methyl-N-(4-methylpyridin-2-yl)-4-(1H-pyrazol-4-yl)thiazol-2-amine
N-(3,5-Difluoropyridin-2-yl)-5-methyl-4-(1H-pyrazol-4-yl)thiazol-2-amine
4-(3-Methyl-1H-pyrazol-4-yl)-N-(4-methylpyridin-2-yl)thiazol-2-amine
5-Ethyl-4-(1H-pyrazol-4-yl)-N-(pyridin-2-yl)thiazol-2-amine
4-(3-Methyl-1H-pyrazol-4-yl)-N-(6-methylpyridin-2-yl)thiazol-2-amine
N-(5-Fluoropyridin-2-yl)-5-methyl-4-(1H-pyrazol-4-yl)thiazol-2-amine
4-(1H-Pyrazol-4-yl)-N-(pyridin-2-yl)thiazol-2-amine
N-(6-Chloropyridin-2-yl)-5-methyl-4-(1H-pyrazol-4-yl)thiazol-2-amine
5-Chloro-4-(1H-pyrazol-4-yl)-N-(pyridin-2-yl)thiazol-2-amine
N-(6-Fluoropyridin-2-yl)-5-methyl-4-(1H-pyrazol-4-yl)thiazol-2-amine
5-Methyl-N-(pyrazin-2-yl)-4-(1H-pyrazol-4-yl)thiazol-2-amine
N-(3-Fluoropyridin-2-yl)-5-methyl-4-(1H-pyrazol-4-yl)thiazol-2-amine
4-(1H-Pyrazol-4-yl)-2-(pyridin-2-ylamino)thiazol-5-carbonitrile
N-(6-Ethylpyridin-2-yl)-5-methyl-4-(1H-pyrazol-4-yl)thiazol-2-amine
N-(6-Chloropyridin-2-yl)-4-(1H-pyrazol-4-yl)thiazol-2-amine
N-(6-Fluoropyridin-2-yl)-4-(1H-pyrazol-4-yl)thiazol-2-amine
5-Chloro-N-(6-methylpyridin-2-yl)-4-(1H-pyrazol-4-yl)thiazol-2-amine
N-(6-Methylpyridin-2-yl)-4-(1H-pyrazol-4-yl)thiazol-2-amine
4-(1H-Pyrazol-4-yl)-N-(pyridin-2-yl)-5-(trifluoromethyl)thiazol-2-amine
5-Phenyl-4-(1H-pyrazol-4-yl)-N-(pyridin-2-yl)thiazol-2-amine
5-Fluoro-4-(1H-pyrazol-4-yl)-N-(pyridin-2-yl)thiazol-2-amine
(4-(1H-Pyrazol-4-yl)-2-(pyridin-2-ylamino)thiazol-5-yl)methanol
N-(6-Methoxypyridin-2-yl)-5-methyl-4-(1H-pyrazol-4-yl)thiazol-2-amine
4-(3-Cyclopropyl-1H-pyrazol-4-yl)-N-(pyridin-2-yl)thiazol-2-amine
4-(3-Ethyl-1H-pyrazol-4-yl)-N-(pyridin-2-yl)thiazol-2-amine;
   provided that according to proviso (ii) the compound is not:
6-(5-Methyl-4-(1H-pyrazol-4-yl)thiazol-2-ylamino)picolinonitrile
5-Morpholino-4-(1H-pyrazol-4-yl)-N-(pyridin-2-yl)thiazol-2-amine
2-(5-Methyl-4-(1H-pyrazol-4-yl)thiazol-2-ylamino)nicotinonitrile
5-(Piperidin-1-yl)-4-(1H-pyrazol-4-yl)-N-(pyridin-2-yl)thiazol-2-amine
4-(3-(Furan-2-yl)-1H-pyrazol-4-yl)-N-(pyridin-2-yl)thiazol-2-amine
4-(3-Isobutyl-1H-pyrazol-4-yl)-N-(pyridin-2-yl)thiazol-2-amine
4-(1H-Pyrazol-4-yl)-N-(pyridin-2-yl)-5-(pyrrolidin-1-yl)thiazol-2-amine
5-Fluoro-N-(6-fluoropyridin-2-yl)-4-(1H-pyrazol-4-yl)thiazol-2-amine
5-Fluoro-N-(6-methylpyridin-2-yl)-4-(1H-pyrazol-4-yl)thiazol-2-amine
N⁵,N⁵-Dimethyl-4-(1H-pyrazol-4-yl)-N²-(pyridin-2-yl)thiazole-2,5-diamine
N-(6-Chloropyridin-2-yl)-5-fluoro-4-(1H-pyrazol-4-yl)thiazol-2-amine
N-(6-Iodopyridin-2-yl)-5-methyl-4-(1H-pyrazol-4-yl)thiazol-2-amine
N-(3-Iodopyridin-2-yl)-5-methyl-4-(1H-pyrazol-4-yl)thiazol-2-amine
5-Methyl-4-(1H-pyrazol-4-yl)-N-(pyrimidin-2-yl)thiazol-2-amine
4-(5-Chloro-1H-pyrazol-4-yl)-N-(pyridin-2-yl)thiazol-2-amine
5-(Methoxymethyl)-4-(1H-pyrazol-4-yl)-N-(pyridin-2-yl)thiazol-2-amine
N-(4-Fluoropyridin-2-yl)-5-methyl-4-(1H-pyrazol-4-yl)thiazol-2-amine
5-((Diethylamino)methyl)-4-(1H-pyrazol-4-yl)-N-(pyridin-2-yl)thiazol-2-amine
5-(Morpholinomethyl)-4-(1H-pyrazol-4-yl)-N-(pyridin-2-yl)thiazol-2-amine
5-(Ethoxymethyl)-4-(1H-pyrazol-4-yl)-N-(pyridin-2-yl)thiazol-2-amine
N-(3-Fluoro-6-methylpyridin-2-yl)-5-methyl-4-(1H-pyrazol-4-yl)thiazol-2-amine
N-(5-Methoxypyridin-2-yl)-5-methyl-4-(1H-pyrazol-4-yl)thiazol-2-amine
5-Fluoro-N-(pyrazin-2-yl)-4-(1H-pyrazol-4-yl)thiazol-2-amine
4-(2-(Pyridin-2-ylamino)thiazol-4-yl)-1H-pyrazole-5-carbonitrile
N-(1-Methyl-1H-pyrazol-3-yl)-4-(1H-pyrazol-4-yl)thiazol-2-amine
5-Fluoro-N-(5-fluoropyridin-2-yl)-4-(1H-pyrazol-4-yl)thiazol-2-amine
N-(Pyridin-2-yl)-4-(3-(2,2,2-trifluoroethyl)-1H-pyrazol-4-yl)thiazol-2-amine
N-(4-Methylpyrimidin-2-yl)-4-(1H-pyrazol-4-yl)thiazol-2-amine
5-Methyl-N-(4-methylpyrimidin-2-yl)-4-(1H-pyrazol-4-yl)thiazol-2-amine
5-Methyl-N-(5-methylpyrimidin-2-yl)-4-(1H-pyrazol-4-yl)thiazol-2-amine
4-(3-Fluoro-1H-pyrazol-4-yl)-N-(pyridin-2-yl)thiazol-2-amine
4-(3-Phenyl-1H-pyrazol-4-yl)-N-(pyridin-2-yl)thiazol-2-amine
5-Methyl-4-(1H-pyrazol-4-yl)-N-(pyrimidin-4-yl)thiazol-2-amine
4-(3-(Phenylsulfonyl)-1H-pyrazol-4-yl)-N-(pyridin-2-yl)thiazol-2-amine
4-(3-(Methoxymethyl)-1H-pyrazol-4-yl)-N-(pyridin-2-yl)thiazol-2-amine
N-(2-Methylthiazol-4-yl)-4-(1H-pyrazol-4-yl)thiazol-2-amine
4-(1H-Pyrazol-4-yl)-N-(thiazol-2-yl)thiazol-2-amine
N-(6-(Fluoromethyl)pyridin-2-yl)-5-methyl-4-(1H-pyrazol-4-yl)thiazol-2-amine
N-(6-(Difluoromethyl)pyridin-2-yl)-5-methyl-4-(1H-pyrazol-4-yl)thiazol-2-amine
5-Chloro-4-(1H-pyrazol-4-yl)-N-(pyrimidin-2-yl)thiazol-2-amine
N-(4-Ethylpyrimidin-2-yl)-5-methyl-4-(1H-pyrazol-4-yl)thiazol-2-amine
N-(5-Fluoropyrimidin-2-yl)-5-methyl-4-(1H-pyrazol-4-yl)thiazol-2-amine
4-(5-(Dimethylamino)-1H-pyrazol-4-yl)-N-(pyridin-2-yl)thiazol-2-amine
5-Chloro-N-(4-methylpyrimidin-2-yl)-4-(1H-pyrazol-4-yl)thiazol-2-amine
4-(1H-Pyrazol-4-yl)-N-(pyrimidin-2-yl)-5-(trifluoromethyl)thiazol-2-amine
N-(4-Isopropylpyrimidin-2-yl)-5-methyl-4-(1H-pyrazol-4-yl)thiazol-2-amine
N-(4-Methoxypyrimidin-2-yl)-4-(1H-pyrazol-4-yl)thiazol-2-amine
1-(4-(2-(4-Methylpyrimidin-2-ylamino)thiazol-4-yl)-1H-pyrazol-5-yl)ethanone
N-(5-Fluoropyrimidin-2-yl)-4-(1H-pyrazol-4-yl)thiazol-2-amine
4-(5-(Dimethylamino)-1H-pyrazol-4-yl)-N-(4-methylpyrimidin-2-yl)thiazol-2-amine
N-(4-Methylpyrimidin-2-yl)-4-(3-(piperidin-1-yl)-1H-pyrazol-4-yl)thiazol-2-amine
N-(4-Methylpyrimidin-2-yl)-4-(1H-pyrazol-4-yl)-5-(trifluoromethyl)thiazol-2-amine
4-(1H-Pyrazol-4-yl)-N-(pyrimidin-2-yl)thiazol-2-amine
N-(4-Methylpyrimidin-2-yl)-4-(3-morpholino-1H-pyrazol-4-yl)thiazol-2-amine
N-(4-Cyclopropylpyrimidin-2-yl)-5-methyl-4-(1H-pyrazol-4-yl)thiazol-2-amine
4-(5-(Diethylamino)-1H-pyrazol-4-yl)-N-(4-methylpyrimidin-2-yl)thiazol-2-amine
4-(5-(Dimethylamino)-1H-pyrazol-4-yl)-N-(pyrimidin-2-yl)thiazol-2-amine
Cyclopropyl(4-(2-(pyrimidin-2-ylamino)thiazol-4-yl)-1H-pyrazol-5-yl)methanone
1-(4-(2-(Pyrimidin-2-ylamino)thiazol-4-yl)-1H-pyrazol-5-yl)ethanone
N⁵-(2-Methoxyethyl)-N⁵-methyl-4-(1H-pyrazol-4-yl)-N²-(pyrimidin-2-yl)thiazole-2,5-diamine
N-(5-Fluoro-4-methylpyrimidin-2-yl)-4-(1H-pyrazol-4-yl)thiazol-2-amine
N-(5-Fluoro-4-methylpyrimidin-2-yl)-5-methyl-4-(1H-pyrazol-4-yl)thiazol-2-amine
4-(3-(Ethyl(methyl)amino)-1H-pyrazol-4-yl)-N-(pyrimidin-2-yl)thiazol-2-amine
4-(3-(Methylamino)-1H-pyrazol-4-yl)-N-(pyrimidin-2-yl)thiazol-2-amine
4-(5-(4-Fluorophenyl)-1H-pyrazol-4-yl)-N-(pyrimidin-2-yl)thiazol-2-amine
4-(5-(Methoxymethyl)-1H-pyrazol-4-yl)-N-(pyrimidin-2-yl)thiazol-2-amine
5-Cyclobutyl-4-(1H-pyrazol-4-yl)-N-(pyrimidin-2-yl)thiazol-2-amine
N-(Pyrimidin-2-yl)-4-(3-(pyrrolidin-1-yl)-1H-pyrazol-4-yl)thiazol-2-amine
4-(3-((2-Methoxyethyl)(methyl)amino)-1H-pyrazol-4-yl)-N-(pyrimidin-2-yl)thiazol-2-amine
N-(4-Methylpyrimidin-2-yl)-4-(3-(pyrrolidin-1-yl)-1H-pyrazol-4-yl)thiazol-2-amine
N-(4-Methylpyrimidin-2-yl)-4-(3-phenyl-1H-pyrazol-4-yl)thiazol-2-amine
(4-(2-(4-Methylpyrimidin-2-ylamino)thiazol-4-yl)-1H-pyrazol-3-yl)methanol
4-(3-(Methoxymethyl)-1H-pyrazol-4-yl)-N-(4-methylpyrimidin-2-yl)thiazol-2-amine
4-(3-(Ethylamino)-1H-pyrazol-4-yl)-N-(4-methylpyrimidin-2-yl)thiazol-2-amine and
4-(3-(2-Methoxyethyl)-1H-pyrazol-4-yl)-N-(4-methylpyrimidin-2-yl)thiazol-2-amine.

In a more preferred aspect of Formula (I), the invention provides a compound wherein:

A radical is selected from the group of hydrogen, halogen, —CN, —CF₃, and an optionally substituted radical selected from the group of —(C₁-C₆)alkyl, —(C₁-C₆)haloalkyl, —(C₃-C₇)cycloalkyl, —(C₁-C₆)cyanoalkyl, heterocycle, heteroaryl, aryl, —(C₀-C₆)alkyl-OR¹, —NR¹(C₂-C₆)alkylene-OR² and —(C₀-C₆)alkyl-NR¹R²;
R¹ and R² are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₄-C₁₀)alkylene-cycloalkyl, heteroaryl, —(C₁-C₆)alkylene-heteroaryl, aryl, heterocycle, —(C₁-C₆)alkylene-heterocycle and —(C₁-C₆)alkylene-aryl;
Any two radicals of R (R¹ or R²) may be taken together to form an optionally substituted 3 to 10 membered carbocyclic or heterocyclic ring;
B radical is selected from the group of hydrogen, halogen, —CN, —CF₃, and an optionally substituted radical selected from the group of —(C₁-C₆)alkyl, —(C₁-C₆)haloalkyl, —(C₃-C₇)cycloalkyl, aryl, —(C₁-C₆)alkylene-aryl, heterocycle, —(C₀-C₆)alkyl-OR⁴, —NR⁴(C₂-C₆)allylene-OR⁵, —(C₀-C₆)alkyl-NR⁴R⁵, —C(═O)—NR⁴R⁵, —(C₀-C₆)alkyl-S(═O)₂—R⁴, —(C₀-C₆)allyl-C(═O)—R⁴, heteroaryl and aryl-(C₁-C₆)alkylene-heterocycle;
R⁴ and R⁵ are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₄-C₁₀)alkylene-cyc loalkyl, heteroaryl, —(C₁-C₆)alkylene-heteroaryl, aryl, heterocycle, —(C₁-C₆)alkylene-heterocycle and —(C₁-C₆)alkylene-aryl; and,
Any two radicals of R (R⁴ or R⁵) may be taken together to form an optionally substituted 3 to 10 membered carbocyclic or heterocyclic ring;
M is an optionally substituted heteroaryl;
provided that according to proviso (i) the compound is not:
4-(3-Methyl-1H-pyrazol-4-yl)-N-(pyridin-2-yl)thiazol-2-amine
N-(Pyridin-2-yl)-4-(3-(trifluoromethyl)-1H-pyrazol-4-yl)thiazol-2-amine
4-(3-Isopropyl-1H-pyrazol-4-yl)-N-(pyridin-2-yl)thiazol-2-amine
5-Methyl-4-(3-methyl-1H-pyrazol-4-yl)-N-(pyridin-2-yl)thiazol-2-amine
5-Methyl-4-(1H-pyrazol-4-yl)-N-(pyridin-2-yl)thiazol-2-amine
5-Methyl-N-(6-methylpyridin-2-yl)-4-(1H-pyrazol-4-yl)thiazol-2-amine
5-Methyl-N-(4-methylpyridin-2-yl)-4-(1H-pyrazol-4-yl)thiazol-2-amine
N-(3,5-Difluoropyridin-2-yl)-5-methyl-4-(1H-pyrazol-4-yl)thiazol-2-amine
4-(3-Methyl-1H-pyrazol-4-yl)-N-(4-methylpyridin-2-yl)thiazol-2-amine
5-Ethyl-4-(1H-pyrazol-4-yl)-N-(pyridin-2-yl)thiazol-2-amine
4-(3-Methyl-1H-pyrazol-4-yl)-N-(6-methylpyridin-2-yl)thiazol-2-amine
N-(5-Fluoropyridin-2-yl)-5-methyl-4-(1H-pyrazol-4-yl)thiazol-2-amine
4-(1H-Pyrazol-4-yl)-N-(pyridin-2-yl)thiazol-2-amine
N-(6-Chloropyridin-2-yl)-5-methyl-4-(1H-pyrazol-4-yl)thiazol-2-amine
5-Chloro-4-(1H-pyrazol-4-yl)-N-(pyridin-2-yl)thiazol-2-amine
N-(6-Fluoropyridin-2-yl)-5-methyl-4-(1H-pyrazol-4-yl)thiazol-2-amine
5-Methyl-N-(pyrazin-2-yl)-4-(1H-pyrazol-4-yl)thiazol-2-amine
N-(3-Fluoropyridin-2-yl)-5-methyl-4-(1H-pyrazol-4-yl)thiazol-2-amine
4-(1H-Pyrazol-4-yl)-2-(pyridin-2-ylamino)thiazol-5-carbonitrile
N-(6-Ethylpyridin-2-yl)-5-methyl-4-(1H-pyrazol-4-yl)thiazol-2-amine
N-(6-Chloropyridin-2-yl)-4-(1H-pyrazol-4-yl)thiazol-2-amine
N-(6-Fluoropyridin-2-yl)-4-(1H-pyrazol-4-yl)thiazol-2-amine
5-Chloro-N-(6-methylpyridin-2-yl)-4-(1H-pyrazol-4-yl)thiazol-2-amine
N-(6-Methylpyridin-2-yl)-4-(1H-pyrazol-4-yl)thiazol-2-amine
4-(1H-Pyrazol-4-yl)-N-(pyridin-2-yl)-5-(trifluoromethyl)thiazol-2-amine
5-Phenyl-4-(1H-pyrazol-4-yl)-N-(pyridin-2-yl)thiazol-2-amine
5-Fluoro-4-(1H-pyrazol-4-yl)-N-(pyridin-2-yl)thiazol-2-amine
(4-(1H-Pyrazol-4-yl)-2-(pyridin-2-ylamino)thiazol-5-yl)methanol
N-(6-Methoxypyridin-2-yl)-5-methyl-4-(1H-pyrazol-4-yl)thiazol-2-amine
4-(3-Cyclopropyl-1H-pyrazol-4-yl)-N-(pyridin-2-yl)thiazol-2-amine and
4-(3-Ethyl-1H-pyrazol-4-yl)-N-(pyridin-2-yl)thiazol-2-amine;
   provided that according to proviso (iii) the compound is not:
6-(5-Methyl-4-(1H-pyrazol-4-yl)thiazol-2-ylamino)picolinonitrile
5-Morpholino-4-(1H-pyrazol-4-yl)-N-(pyridin-2-yl)thiazol-2-amine
2-(5-Methyl-4-(1H-pyrazol-4-yl)thiazol-2-ylamino)nicotinonitrile
5-(Piperidin-1-yl)-4-(1H-pyrazol-4-yl)-N-(pyridin-2-yl)thiazol-2-amine
4-(3-(Furan-2-yl)-1H-pyrazol-4-yl)-N-(pyridin-2-yl)thiazol-2-amine
4-(3-Isobutyl-1H-pyrazol-4-yl)-N-(pyridin-2-yl)thiazol-2-amine
4-(1H-Pyrazol-4-yl)-N-(pyridin-2-yl)-5-(pyrrolidin-1-yl)thiazol-2-amine
5-Fluoro-N-(6-fluoropyridin-2-yl)-4-(1H-pyrazol-4-yl)thiazol-2-amine
5-Fluoro-N-(6-methylpyridin-2-yl)-4-(1H-pyrazol-4-yl)thiazol-2-amine
N⁵,N⁵-Dimethyl-4-(1H-pyrazol-4-yl)-N²-(pyridin-2-yl)thiazole-2,5-diamine
N-(6-Chloropyridin-2-yl)-5-fluoro-4-(1H-pyrazol-4-yl)thiazol-2-amine
N-(6-Iodopyridin-2-yl)-5-methyl-4-(1H-pyrazol-4-yl)thiazol-2-amine
N-(3-Iodopyridin-2-yl)-5-methyl-4-(1H-pyrazol-4-yl)thiazol-2-amine
5-Methyl-4-(1H-pyrazol-4-yl)-N-(pyrimidin-2-yl)thiazol-2-amine
4-(5-Chloro-1H-pyrazol-4-yl)-N-(pyridin-2-yl)thiazol-2-amine
5-(Methoxymethyl)-4-(1H-pyrazol-4-yl)-N-(pyridin-2-yl)thiazol-2-amine
N-(4-Fluoropyridin-2-yl)-5-methyl-4-(1H-pyrazol-4-yl)thiazol-2-amine
5-((Diethylamino)methyl)-4-(1H-pyrazol-4-yl)-N-(pyridin-2-yl)thiazol-2-amine
5-(Ethoxymethyl)-4-(1H-pyrazol-4-yl)-N-(pyridin-2-yl)thiazol-2-amine
N-(3-Fluoro-6-methylpyridin-2-yl)-5-methyl-4-(1H-pyrazol-4-yl)thiazol-2-amine
N-(5-Methoxypyridin-2-yl)-5-methyl-4-(1H-pyrazol-4-yl)thiazol-2-amine
5-Fluoro-N-(pyrazin-2-yl)-4-(1H-pyrazol-4-yl)thiazol-2-amine
4-(2-(Pyridin-2-ylamino)thiazol-4-yl)-1H-pyrazole-5-carbonitrile
N-(1-Methyl-1H-pyrazol-3-yl)-4-(1H-pyrazol-4-yl)thiazol-2-amine
5-Fluoro-N-(5-fluoropyridin-2-yl)-4-(1H-pyrazol-4-yl)thiazol-2-amine
N-(Pyridin-2-yl)-4-(3-(2,2,2-trifluoroethyl)-1H-pyrazol-4-yl)thiazol-2-amine
N-(4-Methylpyrimidin-2-yl)-4-(1H-pyrazol-4-yl)thiazol-2-amine
5-Methyl-N-(4-methylpyrimidin-2-yl)-4-(1H-pyrazol-4-yl)thiazol-2-amine
5-Methyl-N-(5-methylpyrimidin-2-yl)-4-(1H-pyrazol-4-yl)thiazol-2-amine
4-(3-Fluoro-1H-pyrazol-4-yl)-N-(pyridin-2-yl)thiazol-2-amine
4-(3-Phenyl-1H-pyrazol-4-yl)-N-(pyridin-2-yl)thiazol-2-amine
5-Methyl-4-(1H-pyrazol-4-yl)-N-(pyrimidin-4-yl)thiazol-2-amine
4-(3-(Phenylsulfonyl)-1H-pyrazol-4-yl)-N-(pyridin-2-yl)thiazol-2-amine
4-(3-(Methoxymethyl)-1H-pyrazol-4-yl)-N-(pyridin-2-yl)thiazol-2-amine
N-(2-Methylthiazol-4-yl)-4-(1H-pyrazol-4-yl)thiazol-2-amine
4-(1H-Pyrazol-4-yl)-N-(thiazol-2-yl)thiazol-2-amine
N-(6-(Fluoromethyl)pyridin-2-yl)-5-methyl-4-(1H-pyrazol-4-yl)thiazol-2-amine
N-(6-(Difluoromethyl)pyridin-2-yl)-5-methyl-4-(1H-pyrazol-4-yl)thiazol-2-amine
5-Chloro-4-(1H-pyrazol-4-yl)-N-(pyrimidin-2-yl)thiazol-2-amine
N-(4-Ethylpyrimidin-2-yl)-5-methyl-4-(1H-pyrazol-4-yl)thiazol-2-amine
N-(5-Fluoropyrimidin-2-yl)-5-methyl-4-(1H-pyrazol-4-yl)thiazol-2-amine
4-(5-(Dimethylamino)-1H-pyrazol-4-yl)-N-(pyridin-2-yl)thiazol-2-amine
5-Chloro-N-(4-methylpyrimidin-2-yl)-4-(1H-pyrazol-4-yl)thiazol-2-amine
4-(1H-Pyrazol-4-yl)-N-(pyrimidin-2-yl)-5-(trifluoromethyl)thiazol-2-amine
N-(4-Isopropylpyrimidin-2-yl)-5-methyl-4-(1H-pyrazol-4-yl)thiazol-2-amine
N-(4-Methoxypyrimidin-2-yl)-4-(1H-pyrazol-4-yl)thiazol-2-amine
1-(4-(2-(4-Methylpyrimidin-2-ylamino)thiazol-4-yl)-1H-pyrazol-5-yl)ethanone
N-(5-Fluoropyrimidin-2-yl)-4-(1H-pyrazol-4-yl)thiazol-2-amine
4-(5-(Dimethylamino)-1H-pyrazol-4-yl)-N-(4-methylpyrimidin-2-yl)thiazol-2-amine
N-(4-Methylpyrimidin-2-yl)-4-(3-(piperidin-1-yl)-1H-pyrazol-4-yl)thiazol-2-amine
N-(4-Methylpyrimidin-2-yl)-4-(1H-pyrazol-4-yl)-5-(trifluoromethyl)thiazol-2-amine
4-(1H-Pyrazol-4-yl)-N-(pyrimidin-2-yl)thiazol-2-amine
N-(4-Methylpyrimidin-2-yl)-4-(3-morpholino-1H-pyrazol-4-yl)thiazol-2-amine
N-(4-Cyclopropylpyrimidin-2-yl)-5-methyl-4-(1H-pyrazol-4-yl)thiazol-2-amine
4-(5-(Diethylamino)-1H-pyrazol-4-yl)-N-(4-methylpyrimidin-2-yl)thiazol-2-amine
4-(5-(Dimethylamino)-1H-pyrazol-4-yl)-N-(pyrimidin-2-yl)thiazol-2-amine
Cyclopropyl(4-(2-(pyrimidin-2-ylamino)thiazol-4-yl)-1H-pyrazol-5-yl)methanone
1-(4-(2-(Pyrimidin-2-ylamino)thiazol-4-yl)-1H-pyrazol-5-yl)ethanone
N⁵-(2-Methoxyethyl)-N⁵-methyl-4-(1H-pyrazol-4-yl)-N²-(pyrimidin-2-yl)thiazole-2,5-diamine
N-(5-Fluoro-4-methylpyrimidin-2-yl)-4-(1H-pyrazol-4-yl)thiazol-2-amine
N-(5-Fluoro-4-methylpyrimidin-2-yl)-5-methyl-4-(1H-pyrazol-4-yl)thiazol-2-amine
4-(3-(Ethyl(methyl)amino)-1H-pyrazol-4-yl)-N-(pyrimidin-2-yl)thiazol-2-amine
4-(3-(Methylamino)-1H-pyrazol-4-yl)-N-(pyrimidin-2-yl)thiazol-2-amine
4-(5-(4-Fluorophenyl)-1H-pyrazol-4-yl)-N-(pyrimidin-2-yl)thiazol-2-amine
4-(5-(Methoxymethyl)-1H-pyrazol-4-yl)-N-(pyrimidin-2-yl)thiazol-2-amine
5-Cyclobutyl-4-(1H-pyrazol-4-yl)-N-(pyrimidin-2-yl)thiazol-2-amine
N-(Pyrimidin-2-yl)-4-(3-(pyrrolidin-1-yl)-1H-pyrazol-4-yl)thiazol-2-amine
4-(3-((2-Methoxyethyl)(methyl)amino)-1H-pyrazol-4-yl)-N-(pyrimidin-2-yl)thiazol-2-amine
N-(4-Methylpyrimidin-2-yl)-4-(3-(pyrrolidin-1-yl)-1H-pyrazol-4-yl)thiazol-2-amine
N-(4-Methylpyrimidin-2-yl)-4-(3-phenyl-1H-pyrazol-4-yl)thiazol-2-amine
(4-(2-(4-Methylpyrimidin-2-ylamino)thiazol-4-yl)-1H-pyrazol-3-yl)methanol
4-(3-(Methoxymethyl)-1H-pyrazol-4-yl)-N-(4-methylpyrimidin-2-yl)thiazol-2-amine
4-(3-(Ethylamino)-1H-pyrazol-4-yl)-N-(4-methylpyrimidin-2-yl)thiazol-2-amine and
4-(3-(2-Methoxyethyl)-1H-pyrazol-4-yl)-N-(4-methylpyrimidin-2-yl)thiazol-2-amine.

In a more preferred aspect of Formula (I), the invention provides a compound wherein:

M is an optionally substituted pyrimidinyl;
provided that according to proviso (iv) the compound is not:
5-Methyl-4-(1H-pyrazol-4-yl)-N-(pyrimidin-2-yl)thiazol-2-amine
N-(4-Methylpyrimidin-2-yl)-4-(1H-pyrazol-4-yl)thiazol-2-amine
5-Methyl-N-(4-methylpyrimidin-2-yl)-4-(1H-pyrazol-4-yl)thiazol-2-amine
5-Methyl-N-(5-methylpyrimidin-2-yl)-4-(1H-pyrazol-4-yl)thiazol-2-amine
5-Methyl-4-(1H-pyrazol-4-yl)-N-(pyrimidin-4-yl)thiazol-2-amine
5-Chloro-4-(1H-pyrazol-4-yl)-N-(pyrimidin-2-yl)thiazol-2-amine
N-(4-Ethylpyrimidin-2-yl)-5-methyl-4-(1H-pyrazol-4-yl)thiazol-2-amine
N-(5-Fluoropyrimidin-2-yl)-5-methyl-4-(1H-pyrazol-4-yl)thiazol-2-amine
5-Chloro-N-(4-methylpyrimidin-2-yl)-4-(1H-pyrazol-4-yl)thiazol-2-amine
4-(1H-Pyrazol-4-yl)-N-(pyrimidin-2-yl)-5-(trifluoromethyl)thiazol-2-amine
N-(4-Isopropylpyrimidin-2-yl)-5-methyl-4-(1H-pyrazol-4-yl)thiazol-2-amine
N-(4-Methoxypyrimidin-2-yl)-4-(1H-pyrazol-4-yl)thiazol-2-amine
1-(4-(2-(4-Methylpyrimidin-2-ylamino)thiazol-4-yl)-1H-pyrazol-5-yl)ethanone
N-(5-Fluoropyrimidin-2-yl)-4-(1H-pyrazol-4-yl)thiazol-2-amine
4-(5-(Dimethylamino)-1H-pyrazol-4-yl)-N-(4-methylpyrimidin-2-yl)thiazol-2-amine
N-(4-Methylpyrimidin-2-yl)-4-(3-(piperidin-1-yl)-1H-pyrazol-4-yl)thiazol-2-amine
N-(4-Methylpyrimidin-2-yl)-4-(1H-pyrazol-4-yl)-5-(trifluoromethyl)thiazol-2-amine
4-(1H-Pyrazol-4-yl)-N-(pyrimidin-2-yl)thiazol-2-amine
N-(4-Methylpyrimidin-2-yl)-4-(3-morpholino-1H-pyrazol-4-yl)thiazol-2-amine
N-(4-Cyclopropylpyrimidin-2-yl)-5-methyl-4-(1H-pyrazol-4-yl)thiazol-2-amine
4-(5-(Diethylamino)-1H-pyrazol-4-yl)-N-(4-methylpyrimidin-2-yl)thiazol-2-amine
4-(5-(Dimethylamino)-1H-pyrazol-4-yl)-N-(pyrimidin-2-yl)thiazol-2-amine
Cyclopropyl(4-(2-(pyrimidin-2-ylamino)thiazol-4-yl)-1H-pyrazol-5-yl)methanone
1-(4-(2-(Pyrimidin-2-ylamino)thiazol-4-yl)-1H-pyrazol-5-yl)ethanone
N⁵-(2-Methoxyethyl)-N⁵-methyl-4-(1H-pyrazol-4-yl)-N²-(pyrimidin-2-yl)thiazole-2,5-diamine
N-(5-Fluoro-4-methylpyrimidin-2-yl)-4-(1H-pyrazol-4-yl)thiazol-2-amine
N-(5-Fluoro-4-methylpyrimidin-2-yl)-5-methyl-4-(1H-pyrazol-4-yl)thiazol-2-amine
4-(3-(Ethyl(methyl)amino)-1H-pyrazol-4-yl)-N-(pyrimidin-2-yl)thiazol-2-amine
4-(3-(Methylamino)-1H-pyrazol-4-yl)-N-(pyrimidin-2-yl)thiazol-2-amine
4-(5-(4-Fluorophenyl)-1H-pyrazol-4-yl)-N-(pyrimidin-2-yl)thiazol-2-amine
4-(5-(Methoxymethyl)-1H-pyrazol-4-yl)-N-(pyrimidin-2-yl)thiazol-2-amine
5-Cyclobutyl-4-(1H-pyrazol-4-yl)-N-(pyrimidin-2-yl)thiazol-2-amine
N-(Pyrimidin-2-yl)-4-(3-(pyrrolidin-1-yl)-1H-pyrazol-4-yl)thiazol-2-amine
4-(3-((2-Methoxyethyl)(methyl)amino)-1H-pyrazol-4-yl)-N-(pyrimidin-2-yl)thiazol-2-amine
N-(4-Methylpyrimidin-2-yl)-4-(3-(pyrrolidin-1-yl)-1H-pyrazol-4-yl)thiazol-2-amine
N-(4-Methylpyrimidin-2-yl)-4-(3-phenyl-1H-pyrazol-4-yl)thiazol-2-amine
(4-(2-(4-Methylpyrimidin-2-ylamino)thiazol-4-yl)-1H-pyrazol-3-yl)methanol
4-(3-(Methoxymethyl)-1H-pyrazol-4-yl)-N-(4-methylpyrimidin-2-yl)thiazol-2-amine
4-(3-(Ethylamino)-1H-pyrazol-4-yl)-N-(4-methylpyrimidin-2-yl)thiazol-2-amine and
4-(3-(2-Methoxyethyl)-1H-pyrazol-4-yl)-N-(4-methylpyrimidin-2-yl)thiazol-2-amine.

In a more preferred aspect of Formula (I), the invention provides a compound wherein:

M is an optionally substituted pyrimidinyl;

### A is H;

B radical is selected from the group of phenyl and pyridinyl which can be optionally substituted by hydrogen, halogen, —CN, —OCF₃, —(C₁-C₆)alkyl, —(C₃-C₇)cycloalkyl, —(C₀-C₆)allyl-O—(C₀-C₆)alkyl, —(C₁-C₆)haloalkylene-O—(C₀-C₆)alkyl, —(C₀-C₆)alkyl-N—((C₀-C₆)alkyl)₂, —(C₀-C₆)alkyl-C(═O)—N((C₀-C₆)alkyl)₂, heterocycle and —(C₁-C₆)alkylene-heterocycle;
provided that according to proviso (v) the compound is not:
4-(5-(4-Fluorophenyl)-1H-pyrazol-4-yl)-N-(pyrimidin-2-yl)thiazol-2-amine and
N-(4-Methylpyrimidin-2-yl)-4-(3-phenyl-1H-pyrazol-4-yl)thiazol-2-amine.

In a more preferred aspect of Formula (I), the invention provides a compound wherein:

M is an optionally substituted radical selected from the group of oxadiazolyl, oxazolyl, thiadiazolyl and triazinyl.

Proviso (i) is based on the disclosures of claims 9 and 10 in the patent application WO2009/010455; and

Provisos (ii), (iii), (iv) and (v) are based on the disclosures of claims 8 and 9 in the patent application WO2010/079239.

Particular preferred compounds of the invention are compounds as mentioned in the following list (List of Particular Preferred Compounds), as well as a pharmaceutically acceptable acid or base addition salt thereof, a stereochemically isomeric form thereof and an N-oxide form thereof:
4-(3-(Difluoromethyl)-1H-pyrazol-4-yl)-N-(4-methylpyrimidin-2-yl)thiazol-2-amine
N-(4-Methylpyrimidin-2-yl)-4-(3-(3-(1,1-dioxido-4-thiomorpholinyl)phenyl)-1H-pyrazol-4-yl)thiazol-2-amine
4-(3-(2-Fluorophenyl)-1H-pyrazol-4-yl)-N-(4-methylpyrimidin-2-yl)thiazol-2-amine
3-(4-(2-(4-Methylpyrimidin-2-ylamino)thiazol-4-yl)-1H-pyrazol-3-yl)benzonitrile
4-(3-(5-Fluoro-2-methoxyphenyl)-1H-pyrazol-4-yl)-N-(4-methylpyrimidin-2-yl)thiazol-2-amine
2,2,2-Trifluoro-1-(4-(4-(2-(4-methylpyrimidin-2-ylamino)thiazol-4-yl)-1H-pyrazol-3-yl)phenyl)ethanol
4-(4-(2-(4-Methylpyrimidin-2-ylamino)thiazol-4-yl)-1H-pyrazol-3-yl)isoindolin-1-one
4-(3-(5,6-Dihydro-4H-pyrrolo[1,2-b]pyrazol-3-yl)-1H-pyrazol-4-yl)-N-(4-methylpyrimidin-2-yl)thiazol-2-amine
N-(4-Methylpyrimidin-2-yl)-4-(3-(3-(morpholinomethyl)phenyl)-1H-pyrazol-4-yl)thiazol-2-amine
4-(3-(5-Chloropyridin-3-yl)-1H-pyrazol-4-yl)-N-(4-methylpyrimidin-2-yl)thiazol-2-amine
4-(3-(2-Methoxyphenyl)-1H-pyrazol-4-yl)-N-(4-methylpyrimidin-2-yl)thiazol-2-amine
N-(4-Methylpyrimidin-2-yl)-4-(3-m-tolyl-1H-pyrazol-4-yl)thiazol-2-amine
4-(3-Morpholino-1H-pyrazol-4-yl)-N-(pyridin-2-yl)thiazol-2-amine
2-(5-(4-(2-(4-Methylpyrimidin-2-ylamino)thiazol-4-yl)-1H-pyrazol-3-yl)pyridin-3-yl)propan-2-ol
3-(3-(4-(2-(4-Methylpyrimidin-2-ylamino)thiazol-4-yl)-1H-pyrazol-3-yl)phenyl)propan-1-ol
4-(3-(4-Chlorophenyl)-1H-pyrazol-4-yl)-N-(4-methylpyrimidin-2-yl)thiazol-2-amine
4-(3-(4-Cyclopropylphenyl)-1H-pyrazol-4-yl)-N-(4-methylpyrimidin-2-yl)thiazol-2-amine
N-(4-Methylpyrimidin-2-yl)-4-(3-(pyridin-4-yl)-1H-pyrazol-4-yl)thiazol-2-amine
N-(4-Methylpyrimidin-2-yl)-4-(3-o-tolyl-1H-pyrazol-4-yl)thiazol-2-amine
N-(4-Methylpyrimidin-2-yl)-4-(3-(2-(trifluoromethoxy)phenyl)-1H-pyrazol-4-yl)thiazol-2-amine
4-(3-(2-Chlorophenyl)-1H-pyrazol-4-yl)-N-(4-methylpyrimidin-2-yl)thiazol-2-amine
3-(4-(2-(4-Methylpyrimidin-2-ylamino)thiazol-4-yl)-1H-pyrazol-3-yl)benzamide
4-(3-(2-Ethoxyphenyl)-1H-pyrazol-4-yl)-N-(4-methylpyrimidin-2-yl)thiazol-2-amine
4-(3-(2,4-Difluorophenyl)-1H-pyrazol-4-yl)-N-(4-methylpyrimidin-2-yl)thiazol-2-amine
(4-(2-(4-Methylpyrimidin-2-ylamino)thiazol-4-yl)-1H-pyrazol-3-yl)(phenyl)methanone
(4-(2-(4-Methylpyrimidin-2-ylamino)thiazol-4-yl)-1H-pyrazol-3-yl)(phenyl)methanol
Cyclopropyl(4-(2-(4-methylpyrimidin-2-ylamino)thiazol-4-yl)-1H-pyrazol-5-yl)methanone
4-(5-(Cyclobutoxymethyl)-1H-pyrazol-4-yl)-N-(4-methylpyrimidin-2-yl)thiazol-2-amine
4-(5-(1-Methoxyethyl)-1H-pyrazol-4-yl)-N-(4-methylpyrimidin-2-yl)thiazol-2-amine
4-(3-(3-(Dimethylamino)phenyl)-1H-pyrazol-4-yl)-N-(4-methylpyrimidin-2-yl)thiazol-2-amine
4-(3-(5-Fluoro-2-methoxyphenyl)-1H-pyrazol-4-yl)-N-(pyrimidin-2-yl)thiazol-2-amine
N-Methyl-4-(2-(4-methylpyrimidin-2-ylamino)thiazol-4-yl)-1H-pyrazole-5-carboxamide
1-(3-(4-(2-(4-Methylpyrimidin-2-ylamino)thiazol-4-yl)-1H-pyrazol-3-yl)-5,6-dihydropyridin-1(2H)-yl)ethanone
4-Fluoro-3-(4-(2-(4-methylpyrimidin-2-ylamino)thiazol-4-yl)-1H-pyrazol-5-yl)benzonitrile
2-Fluoro-3-(4-(2-(4-methylpyrimidin-2-ylamino)thiazol-4-yl)-1H-pyrazol-5-yl)benzonitrile
N-(3-Fluoro-6-methylpyridin-2-yl)-4-(3-(methoxymethyl)-1H-pyrazol-4-yl)thiazol-2-amine
N-(5-Fluoropyrimidin-2-yl)-4-(3-(methoxymethyl)-1H-pyrazol-4-yl)thiazol-2-amine
N-(4-Ethylpyrimidin-2-yl)-4-(3-(methoxymethyl)-1H-pyrazol-4-yl)thiazol-2-amine
Methyl 3-(4-(2-(4-methylpyrimidin-2-ylamino)thiazol-4-yl)-1H-pyrazol-3-yl)-2,5-dihydro-1H-pyrrole-1-carboxylate
1-(4-(4-(2-(4-Methylpyrimidin-2-ylamino)thiazol-4-yl)-1H-pyrazol-3-yl)-5,6-dihydropyridin-1(2H)-yl)ethanone
Methyl 4-(4-(2-(4-methylpyrimidin-2-ylamino)thiazol-4-yl)-1H-pyrazol-3-yl)-5,6-dihydropyridine-1 (2H)-carboxylate
1-(3-(4-(2-(4-Methylpyrimidin-2-ylamino)thiazol-4-yl)-1H-pyrazol-3-yl)phenyl)pyrrolidin-3-ol
3-(4-(2-(Pyrimidin-2-ylamino)thiazol-4-yl)-1H-pyrazol-3-yl)benzonitrile
2-(5-Methyl-1,2,4-thiadiazol-3-ylamino)-4-(1H-pyrazol-4-yl)thiazole-5-carbonitrile
2-(1,2,4-Triazin-3-ylamino)-4-(1H-pyrazol-4-yl)thiazole-5-carbonitrile
1-(2-(4-Methylpyrimidin-2-ylamino)-4-(1H-pyrazol-4-yl)thiazol-5-yl)ethanone and
5-Ethoxy-N-(4-methylpyrimidin-2-yl)-4-(1H-pyrazol-4-yl)thiazol-2-amine.

The disclosed compounds also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³²P and ³³P; isotopes of sulfur, such as ³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula (I) can generally be prepared by conventional techniques known to those skilled in the art or by processes analogous to those described in the accompanying Examples using appropriate isotopically-labeled reagents in place of the non-labeled reagent previously employed.

### DEFINITION OF TERMS

Listed below are definitions of various terms used in the specification and claims to describe the present invention.

For the avoidance of doubt it is to be understood that in this specification “(C₁-C₆)” means a carbon radical having 1, 2, 3, 4, 5 or 6 carbon atoms. “(C₀-C₆)” means a carbon radical having 0, 1, 2, 3, 4, 5 or 6 carbon atoms. In this specification “C” means a carbon atom, “N” means a nitrogen atom, “O” means an oxygen atom and “S” means a sulphur atom.

In the case where a subscript is the integer 0 (zero) the radical to which the subscript refers, indicates that the radical is absent, i.e. there is a direct bond between the radicals.

In the case where a subscript is the integer 0 (zero) and the radical to which the subscript refers is alkyl, this indicates the radical is a hydrogen atom.

In this specification, unless stated otherwise, the term “bond” refers to a saturated covalent bond. When two or more bonds are adjacent to one another, they are assumed to be equal to one bond. For example, a radical -A-B-, wherein both A and B may be a bond, the radical is depicting a single bond.

In this specification, unless stated otherwise, the term “alkyl” includes both straight and branched chain alkyl radicals and may be methyl, ethyl, n-propyl, i-propyl, n-butyl, i-butyl, s-butyl, t-butyl, n-pentyl, i-pentyl, t-pentyl, neo-pentyl, n-hexyl, i-hexyl or t-hexyl. The term “(C₀-C₃)alkyl” refers to an alkyl radical having 0, 1, 2 or 3 carbon atoms and may be methyl, ethyl, n-propyl and i-propyl.

In this specification, unless stated otherwise, the term “alkylene” includes both straight and branched difunctional saturated hydrocarbon radicals and may be methylene, ethylene, n-propylene, i-propylene, n-butylene, i-butylene, s-butylene, t-butylene, n-pentylene, i-pentylene, t-pentylene, neo-pentylene, n-hexylene, i-hexylene or t-hexylene.

In this specification, unless stated otherwise, the term “cycloalkyl” refers to an optionally substituted carbocycle containing no heteroatoms, including mono-, bi-, and tricyclic saturated carbocycles, as well as fused ring systems. Such fused ring systems can include one ring that is partially or fully unsaturated such as a benzene ring to form fused ring systems such as benzo-fused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, fluorenyl and 1,2,3,4-tetrahydronaphthalene and the like. The term “(C₃-C₇)cycloalkyl” may be cyclopropyl, cyclobutyl, cyclopentyl, cyclohexyl, cycloheptyl and the like.

The term “aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl, indenyl and the like.

In this specification, unless stated otherwise, the term “heteroaryl” refers to an optionally substituted monocyclic or bicyclic unsaturated, aromatic ring system containing at least one heteroatom selected independently from N, O or S. Examples of “heteroaryl” may be, but are not limited to thienyl, pyridinyl, thiazolyl, isothiazolyl, furyl, pyrrolyl, triazolyl, imidazolyl, triazinyl, oxadiazolyl, oxazolyl, isoxazolyl, pyrazolyl, imidazolonyl, oxazolonyl, thiazolonyl, tetrazolyl, thiadiazolyl, benzoimidazolyl, benzooxazolyl, benzothiazolyl, tetrahydrotriazolopyridinyl, tetrahydrotriazolopyrimidinyl, benzofuryl, benzothiophenyl, thionaphthyl, indolyl, isoindolyl, pyridonyl, pyridazinyl, pyrazinyl, pyrimidinyl, quinolyl, phtalazinyl, naphthyridinyl, quinoxalinyl, quinazolyl, imidazopyridinyl, oxazolopyridinyl, thiazolopyridinyl, imidazopyridazinyl, oxazolopyridazinyl, thiazolopyridazinyl, cynnolyl, pteridinyl, furazanyl, benzotriazolyl, pyrazolopyridinyl and purinyl.

In this specification, unless stated otherwise, the term “alkylene-aryl”, “alkylene-heteroaryl” and “alkylene-cycloalkyl” refers respectively to a substituent that is attached via the alkyl radical to an aryl, heteroaryl or cycloalkyl radical, respectively. The term “(C₁-C₆)alkylene-aryl” includes aryl-C₁-C₆-alkyl radicals such as benzyl, 1-phenylethyl, 2-phenylethyl, 1-phenylpropyl, 2-phenylpropyl, 3-phenylpropyl, 1-naphthylmethyl and 2-naphthylmethyl. The term “(C₁-C₆)alkylene-heteroaryl” includes heteroaryl-C₁-C₆-alkyl radicals, wherein examples of heteroaryl are the same as those illustrated in the above definition, such as 2-furylmethyl, 3-furylmethyl, 2-thienylmethyl, 3-thienylmethyl, 1-imidazo lylmethyl, 2-imidazolylmethyl, 3-imidazolylmethyl, 2-oxazolylmethyl, 3-oxazolylmethyl, 2-thiazolylmethyl, 3-thiazolylmethyl, 2-pyridinylmethyl, 3-pyridinylmethyl, 4-pyridinylmethyl, 1-quinolylmethyl or the like.

In this specification, unless stated otherwise, the term “heterocycle” refers to an optionally substituted, monocyclic or bicyclic saturated, partially saturated or unsaturated ring system containing at least one heteroatom selected independently from N, O and S.

In this specification, unless stated otherwise, a 5- or 6-membered ring containing one or more atoms independently selected from C, N, O and S, includes aromatic and heteroaromatic rings as well as carbocyclic and heterocyclic rings which may be saturated or unsaturated. Examples of such rings may be, but are not limited to, furyl, isoxazolyl, isothiazolyl, oxazolyl, pyrazinyl, pyrazolyl, pyridazinyl, pyridinyl, pyrimidinyl, pyrrolyl, thiazolyl, thienyl, imidazolyl, imidazolidinyl, imidazolinyl, triazolyl, morpholinyl, piperazinyl, piperidyl, piperidonyl, pyrazolidinyl, pyrazolinyl, pyrrolidinyl, pyrrolinyl, tetrahydropyranyl, tetrahydrothiopyranyl, oxazolidinonyl, thiomorpholinyl, oxadiazolyl, thiadiazolyl, tetrazolyl, phenyl, cyclohexyl, cyclopentyl, cyclohexenyl and cyclopentenyl.

In this specification, unless stated otherwise, a 3- to 10-membered ring containing one or more atoms independently selected from C, N, O and S, includes aromatic and heteroaromatic rings as well as carbocyclic and heterocyclic rings which may be saturated or unsaturated. Examples of such rings may be, but are not limited to imidazolidinyl, imidazolinyl, morpholinyl, piperazinyl, piperidyl, piperidonyl, pyrazolidinyl, pyrazolinyl, pyrrolidinyl, pyrrolinyl, tetrahydropyranyl, thiomorpholinyl, tetrahydrothiopyranyl, furyl, pyrrolyl, dihydropyrrolyl isoxazolyl, isothiazolyl, isoindolinonyl, dihydropyrrolo[1,2-b]pyrazolyl, oxazolyl, oxazolidinonyl, pyrazinyl, pyrazolyl, pyridazinyl, pyridinyl, tetrahydropyridinyl, pyrimidinyl, pyrrolyl, thiazolyl, thienyl, imidazolyl, triazolyl, phenyl, cyclopropyl, aziridinyl, cyclobutyl, azetidinyl, oxadiazolyl, thiadiazolyl, tetrazolyl, cyclopentyl, cyclopentenyl, cyclohexyl, cyclohexenyl, cycloheptyl, cycloheptenyl, cyclooctyl and cyclooctenyl.

In this specification, unless stated otherwise, the term “halo” or “halogen” may be fluoro, chloro, bromo or iodo.

In this specification, unless stated otherwise, the term “haloalkyl” means an alkyl radical as defined above, substituted with one or more halo radicals. The term “(C₁-C₆)haloalkyl” may include, but is not limited to, fluoromethyl, difluoromethyl, trifluoromethyl, fluoroethyl and difluoroethyl. The term “O—C₁-C₆-haloalkyl” may include, but is not limited to, fluoromethoxy, difluoromethoxy, trifluoromethoxy and fluoroethoxy.

In this specification, unless stated otherwise, the term “haloalkylene” means an alkylene radical as defined above, substituted with one or more halo radicals. The term “(C₁-C₆)haloalkylene” may include, but is not limited to, fluoromethylene, difluoromethylene, fluoroethylene and difluoroethylene. The term “O—C₁-C₆-haloalkylene” may include, but is not limited to, fluoromethylenoxy, difluoromethylenoxy and fluoroethylenoxy.

In this specification, unless stated otherwise, the term “cyanoalkyl” means an alkyl radical as defined above, substituted with one or more cyano.

In this specification, unless stated otherwise, the term “optionally substituted” refers to radicals further bearing one or more substituents which may be, (C₁-C₆)alkyl, hydroxy, (C₁-C₆)alkylene-oxy, mercapto, aryl, heterocycle, heteroaryl, (C₁-C₆)alkylene-aryl, (C₁-C₆)alkylene-heterocycle, (C₁-C₆)alkylene-heteroaryl, halogen, trifluoromethyl, pentafluoroethyl, cyano, cyanomethyl, nitro, amino, amido, amidinyl, carboxyl, carboxamide, (C₁-C₆)alkylene-oxycarbonyl, carbamate, sulfonamide, ester and sulfonyl.

In this specification, unless stated otherwise, the term “solvate” refers to a complex of variable stoichiometry formed by a solute (e.g. a compound of Formula (I)) and a solvent. The solvent is a pharmaceutically acceptable solvent as preferably water; such solvent may not interfere with the biological activity of the solute.

In this specification, unless stated otherwise, the term “positive allosteric modulator of mGluR4” or “allosteric modulator of mGluR4” refers also to a pharmaceutically acceptable acid or base addition salt thereof, a stereochemically isomeric form thereof and an N-oxide form thereof.

### Pharmaceutical Compositions

Allosteric modulators of mGluR4 described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The allosteric modulators of mGluR4 will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, Pa. (1995).

The amount of allosteric modulators of mGluR4, administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used CNS drugs are well known to the skilled person. The total daily dose usually ranges from about 0.05-2000 mg.

The present invention relates to pharmaceutical compositions which provide from about 0.01 to 1000 mg of the active ingredient per unit dose. The compositions may be administered by any suitable route. For example orally in the form of capsules and the like, parenterally in the form of solutions for injection, topically in the form of onguents or lotions, ocularly in the form of eye-drops, rectally in the form of suppositories, intranasally or transcutaneously in the form of delivery system like patches.

For oral administration, the allosteric modulators of mGluR4 thereof can be combined with a suitable solid or liquid carrier or diluent to form capsules, tablets, pills, powders, syrups, solutions, suspensions and the like.

The tablets, pills, capsules, and the like contain from about 0.01 to about 99 weight percent of the active ingredient and a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parenteral administration the disclosed allosteric modulators of mGluR4 can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition, to the formulations described previously, the compounds may also be formulated as a depot preparation. Such long acting formulations may be administered for example, by subcutaneously implantation or by intramuscular injection. Thus, for example, as an emulsion in an acceptable oil, or ion exchange resins, or as sparingly soluble derivatives, for example, as sparingly soluble salts.

Preferably disclosed allosteric modulators of mGluR4 or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

Classical treatment of Parkinsonism typically involves the use of levodopa combined with carbidopa (SINEMET™) or benserazide (MADOPAR™). Dopamine agonists such as bromocriptine (PARLODEL™), lisuride and pergolide (CELANCE™) act direcly on dopamine receptors and are also used for the treatment of Parkinsonism.

### Methods of Synthesis

The compounds according to the invention, in particular the compounds according to the Formula (I), may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (Green T. W. and Wuts P. G. M., (1991) Protecting Groups in Organic Synthesis, John Wiley & Sons). These groups are removed at a convenient stage of the compound synthesis using methods that are readily apparent to those skilled in the art. The selection of process as well as the reaction conditions and order of their execution shall be consistent with the preparation of compounds of Formula (I).

The compounds according to the invention may be represented as a mixture of enantiomers, which may be resolved into the individual pure R- or S-enantiomers. If for instance, a particular enantiomer is required, it may be prepared by asymmetric synthesis or by derivation with a chiral auxiliary, where the resulting diastereomeric mixture is separated and the auxiliary group cleaved to provide the pure desired enantiomers. Alternatively, where the molecule contains a basic functional group such as an amino or an acidic functional group such as carboxyl, this resolution may be conveniently performed by fractional crystallization from various solvents as the salts of an optical active acid or by other methods known in the literature (e.g. chiral column chromatography).

Resolution of the final product, an intermediate or a starting material may be performed by any suitable method known in the art (Eliel E. L., Wilen S. H. and Mander L. N., (1984) Stereochemistry of Organic Compounds, Wiley-Interscience).

Many of the heterocyclic compounds of the invention can be prepared using synthetic routes well known in the art (Katrizky A. R. and. Rees C. W., (1984) Comprehensive Heterocyclic Chemistry, Pergamon Press).

The product from the reaction can be isolated and purified employing standard techniques, such as extraction, chromatography, crystallization and distillation.

The compounds of the invention may be prepared by general route of synthesis as disclosed in the following methods.

In one embodiment of the present invention compounds of Formula (I) may be prepared according to the synthetic sequences illustrated in Scheme 1. A well known procedure to synthesize pyrazole is from ketoester g1 which is condensed with 1,1-dimethoxy-N,N-dimethylmethanamine followed by cyclisation in the presence of hydrazine. Pyrazole g3 can be protected by p-methoxybenzyl, for example, using standard conditions. Then compound g4 may be hydrolyzed and the resulting carboxylic acid g5 can be transformed into the corresponding Weinreb amide which undergoes a Grignard reagent addition. Subsequently, the resulting ketone can be brominated and submitted to the cyclisation in the presence of thiourea to yield aminothiazole g9 which can be coupled via Buchwald coupling to haloheteroaryl (M-Br) and finally can be deprotected under classical conditions, well known to people skilled in the art, to furnish compound g10.

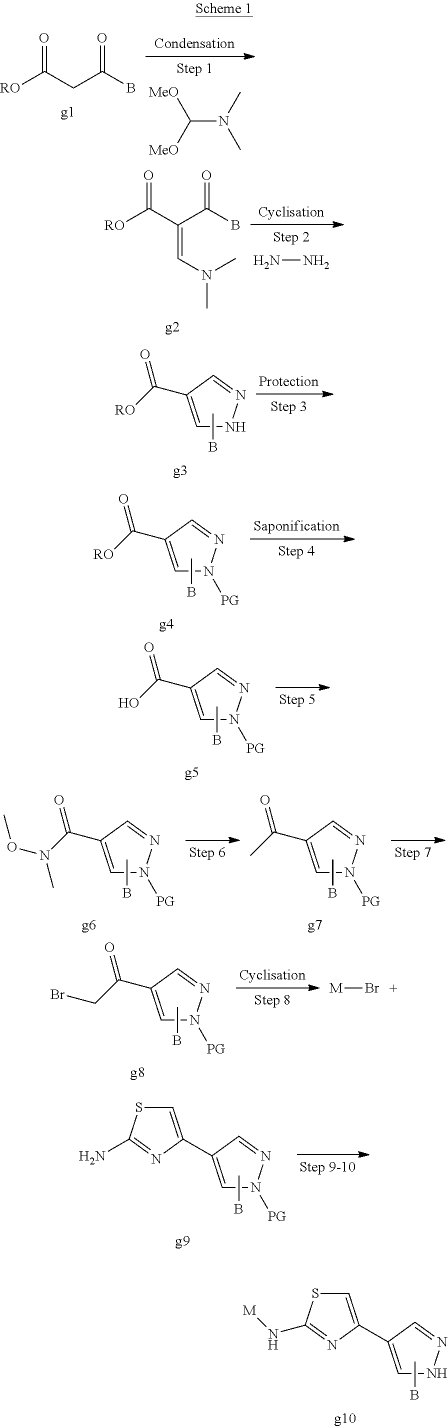

In one embodiment of the present invention, the compounds of Formula (I) may be prepared according to the synthetic sequences illustrated in Scheme 2. Pyrazole g3 can be protected using MOMCl, for example, using standard conditions. Then the ester moiety in g4 may be converted into Weinreb amide g6, in the presence of trimethylaluminium, which undergoes addition of Grignard reagent to yield ketone g7. Subsequently, ketone g7 can be transformed into bromoketone g11 with simultaneous deprotection, in the presence of phenyltrimethylammonium tribromide, and can finally be cyclized into the aminothiazole g10 by reaction with thiourea g12.

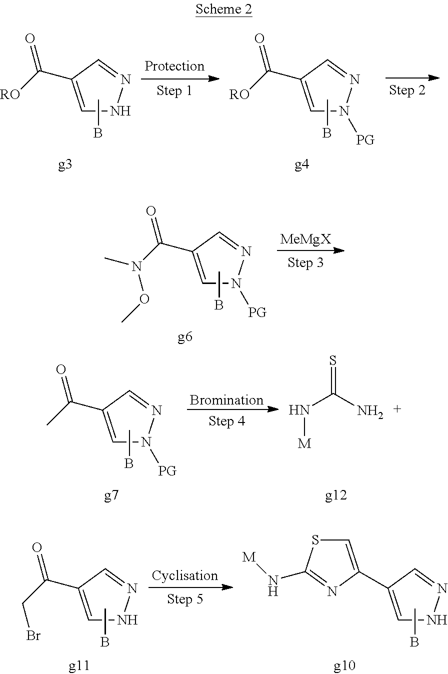

In one embodiment of the present invention, the compounds of Formula (I) may be prepared according to the synthetic sequences illustrated in Scheme 3. Pyrazole g13 can be protected by p-methoxybenzyl, for example, using standard conditions. Then the ester g14 may be subjected to the addition of (cyanomethyl)lithium in order to afford 1,3-cyano-carbonyl compound g15 which undergoes easy bromination, in the presence of cupric bromide. Cyclisation may be performed with thiourea and the resulting 2-aminothiazole g17 transformed into 2-bromothiazole g18 using standard Sandmeyer conditions. g18 is subsequently coupled to MNH₂ via Buchwald coupling and finally deprotected under classical conditions to yield g20.

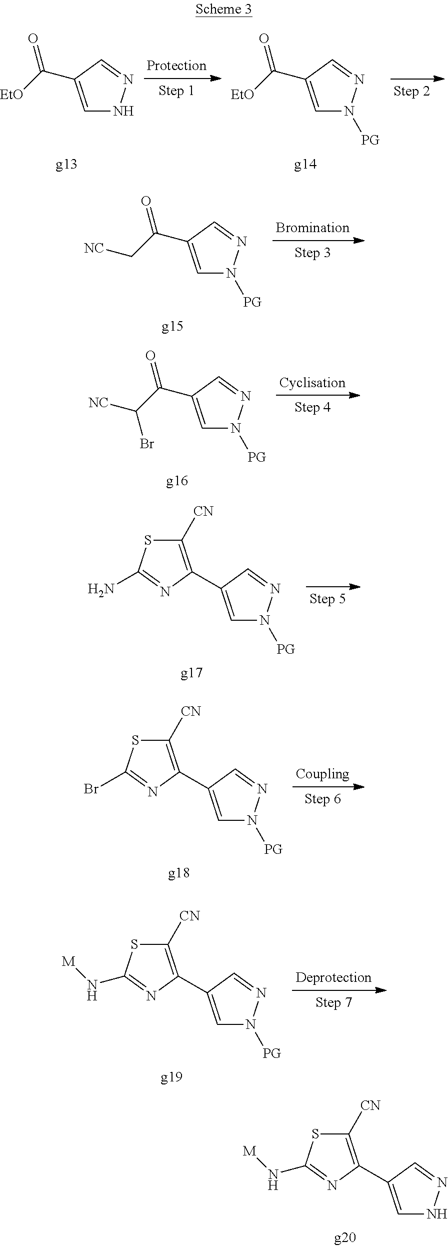

In one embodiment of the present invention, the compounds of Formula (I) may be prepared according to the synthetic sequences illustrated in Scheme 4. Aldehyde moiety can be introduced on pyrazole g21 using standard conditions, like in the presence of LDA and DMF, at −78° C. Then the aldehyde can be fluorinated by DAST in order to generate difluoro compound g23. Weinreb amide g23 can undergo a Grignard addition reaction to yield ketone g24. Subsequently, ketone g24 can be transformed into bromoketone g25 which can be cyclized into the aminothiazole g26 with thiourea g12 and finally deprotected.

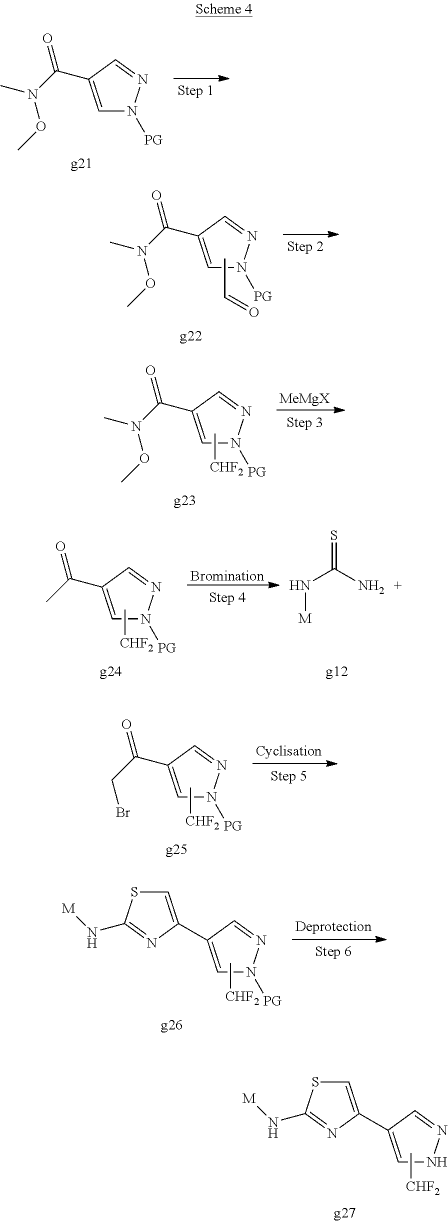

In one embodiment of the present invention, the compounds of Formula (I) may be prepared according to the synthetic sequences illustrated in Scheme 5. Aldehyde moiety can be introduced on pyrazole g14 as described above. The aldehyde can then be reduced to an alcohol and transformed into a good leaving group such as a sulfonate. The compound g30 can be reacted with an alcohol in order to generate the ether g31. The ester moiety of g31 in the presence of trimethylaluminium can afford Weinreb amide g32 which can undergo a Grignard reaction to yield ketone g33. Subsequently, ketone g33 can be transformed into bromoketone g34 with concurrent deprotection using phenyltrimethylammonium tribromide. Finally, g35 can be obtained from the cyclisation of g34 and thiourea g12, in a solvent such as ethanol.

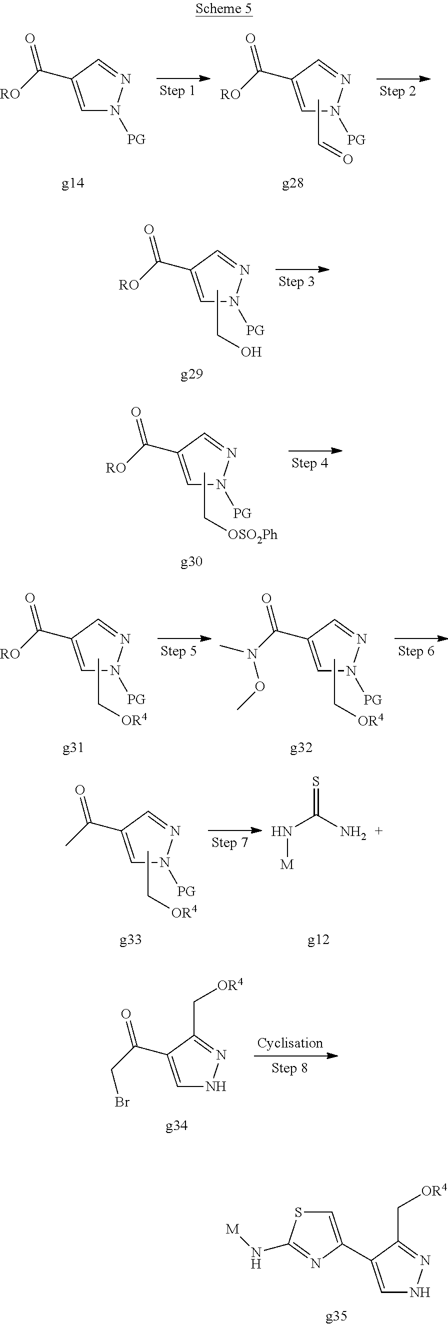

In one embodiment of the present invention, the compounds of Formula (I) may be prepared according to the synthetic sequences illustrated in Scheme 6. 3-Aminopyrazole g36 can be transformed into the corresponding 3-bromopyrazole via a Sandmeyer reaction. Then compound g37 can be protected by p-methoxybenzyl using standard conditions. Hydrolysis of ester g38 and formation of the bromoketone, was carried out via the acid chloride and subsequently the diazoketone. A cyclisation reaction may be performed between thiourea g12 and bromoketone g40 to yield aminothiazole g41. Bromopyrazole g41 may undergo Suzuki coupling using a boronic ester or boronic acid derivative, Pd(PPh₃)₄ as catalyst and a saturated solution of NaHCO₃ as base in a solvent such as dioxane. Then compound g43 can be obtained after deprotection of g42 in the presence of TFA using thermic or microwave conditions.

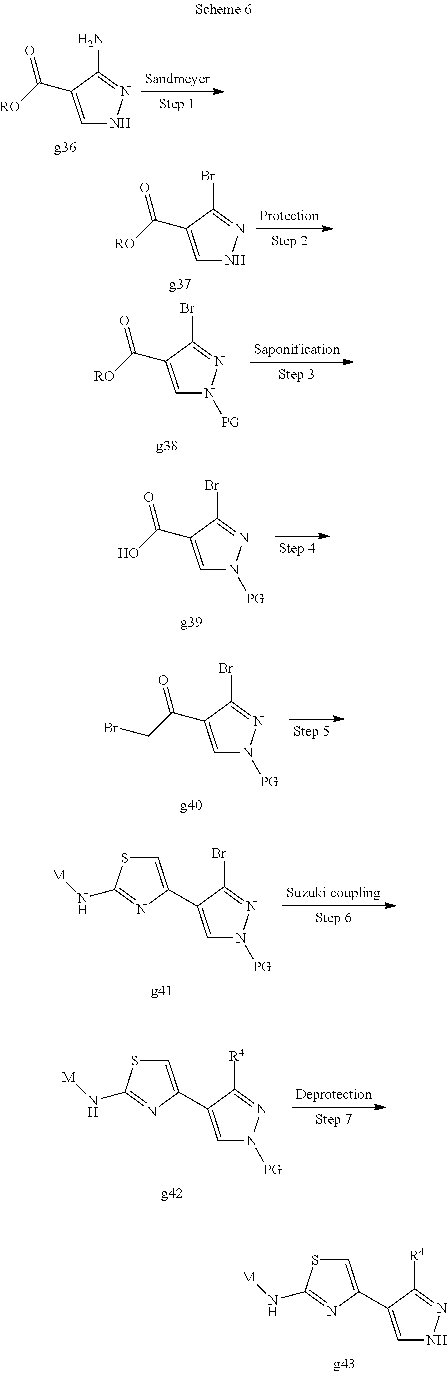

In one embodiment of the present invention, the compounds of Formula (I) may be prepared according to the synthetic sequences illustrated in Scheme 7. Bromoketone g40, described above, can also be cyclized with thiourea, to generate aminothiazole g44. This compound can be protected by two Boc groups and subsequently subjected to Suzuki coupling conditions to yield compound g46. After deprotection of the amino moiety, using acidic conditions, Buchwald coupling can be performed in the presence of MX and finally compound g43 is afforded after deprotection under acidic conditions.

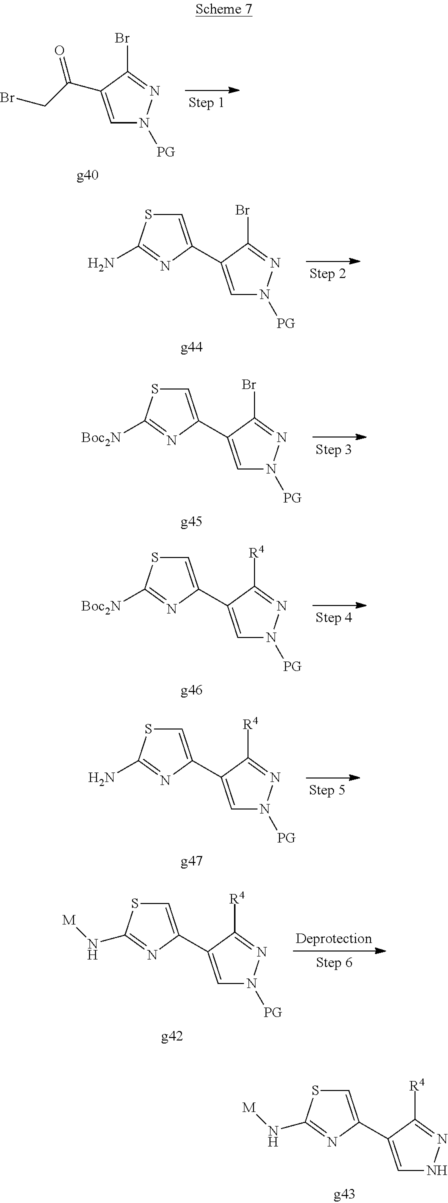

In one embodiment of the present invention, the compounds of Formula (I) may be prepared according to the synthetic sequences illustrated in Scheme 8. Boronic ester g52, can be synthesized by coupling the secondary amine with dibromophenyl. The primary alcohol can be protected with TBSCl and the bromide g51 can be converted to the boronic ester. Boronate g52 can be coupled to bromopyrazole g41 via Suzuki coupling conditions, well known for people skilled in the art, and finally deprotection of the alcohol and the pyrazole can be carried out simultaneously under acidic conditions.

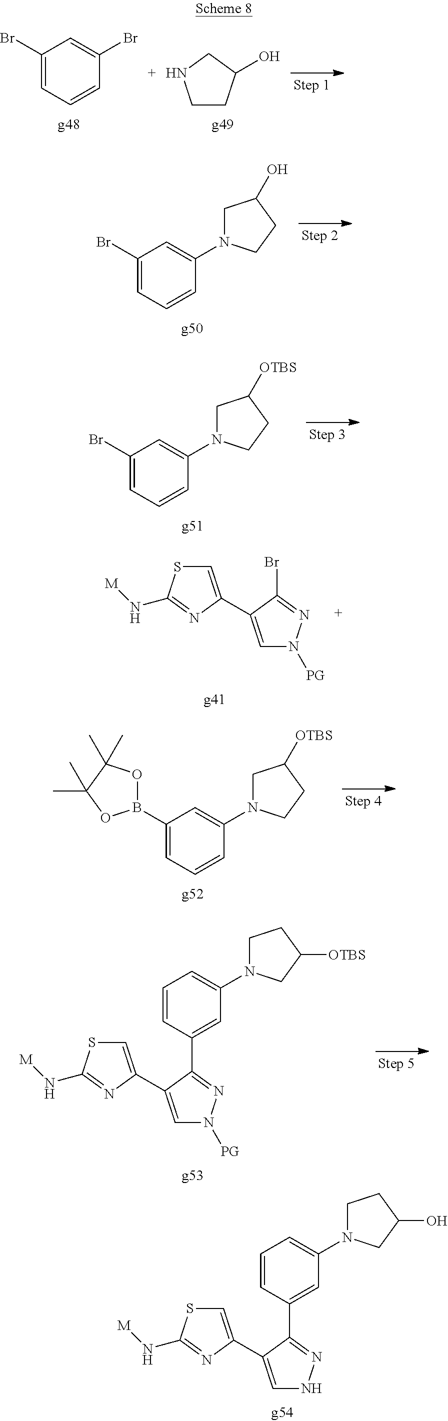

In one embodiment of the present invention, the compounds of Formula (I) may be prepared according to the synthetic sequences illustrated in Scheme 9. Bromopyrazole g41 can also be coupled to five or six-membered heterocycle such as protected dihydropyrrole or tetrahydropyridine. After cleavage of the Boc group with a solution of HCl in MeOH, the secondary amine can then be converted to an amide or carbamate using standard conditions. Finally the compound g58 was obtained by cleaving off the protecting group, for example PMB, using TFA as reagent, under microwave conditions.

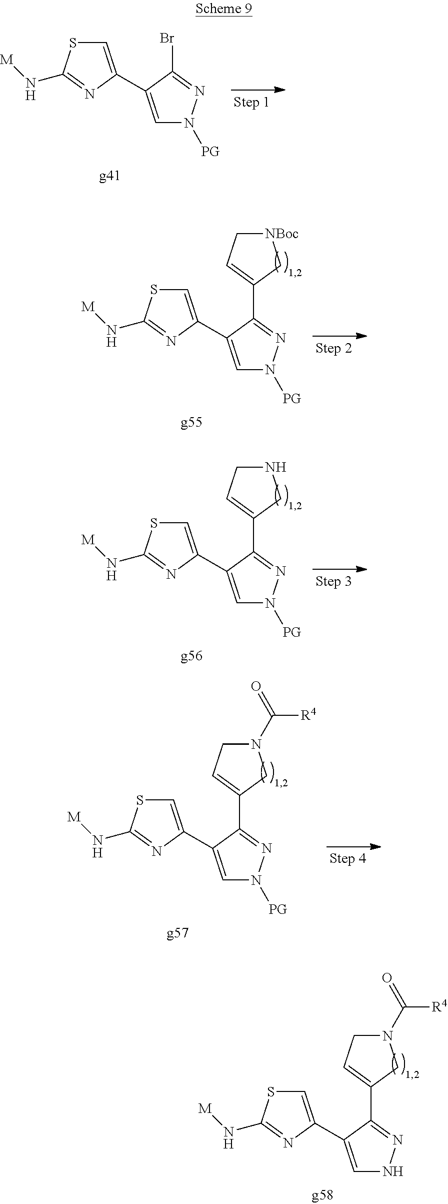

In one embodiment of the present invention, the compounds of Formula (I) may be prepared according to the synthetic sequences illustrated in Scheme 10. Functionalised pyrazole g60 can be obtained from deprotonation of pyrazole g21 using LDA as a base in THF at −78° C. followed by the addition of hexachloroethane. The subsequent chloropyrazole g59 may be substituted by a primary or secondary amine into aminopyrazole g60 which can subsequently be used in the Schemes described above in order to synthesize compounds of Formula (I).

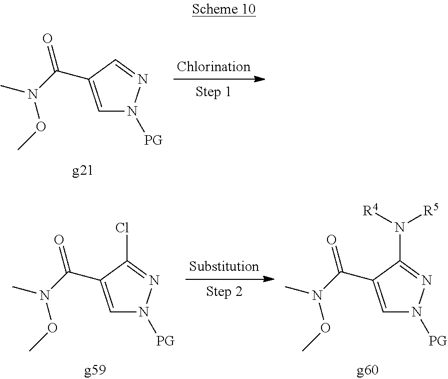

In one embodiment of the present invention, the compounds of Formula (I) may be prepared according to Scheme 11. Functionalised pyrazole g62 can be obtained from deprotonation of pyrazole g14 using LDA as a base in THF at −78° C. followed by the addition of an aldehyde. The resulting alcohol g61 can then be oxidized in the presence of Dess Martin reagent in a solvent such as DCM and the corresponding ketone g62 can subsequently be used in the Schemes described above in order to synthesize compounds of Formula (I).

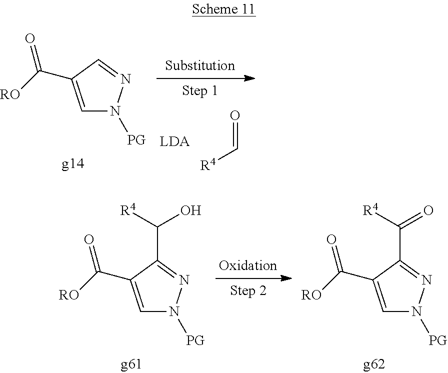

In one embodiment of the present invention, the compounds of Formula (I) may be prepared according to Scheme 12. Compound g63 as described above can be reduced in the presence of LiAlH₄ in a solvent such as THF to yield the alcohol g64.

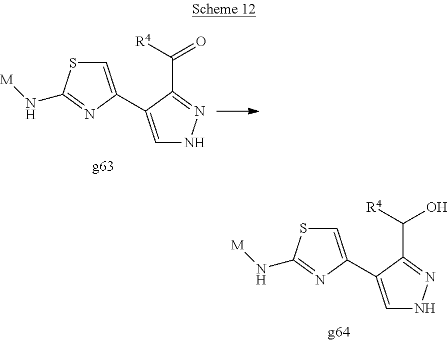

In one embodiment of the present invention, the compounds of Formula (I) may be prepared according to Scheme 13. Iodopyrazole g65 can be protected, for example, by PMBCl. The ester moiety was then saponified, treated with oxalyl chloride and finally with N,O-dimethylhydroxylamine hydrochloride to yield the Weinreb amide g68. After Heck coupling, bromination of the resulting ketone and cyclisation with thiourea, compound g71 was obtained, as described above. Finally Weinreb amide can be transformed into methyl amide via formation of the carboxylic acid which is then treated with methanamine in the presence of coupling agent such as EDCI. The final product g73 can be easily obtained by deprotection of the pyrazole in the presence of TFA.

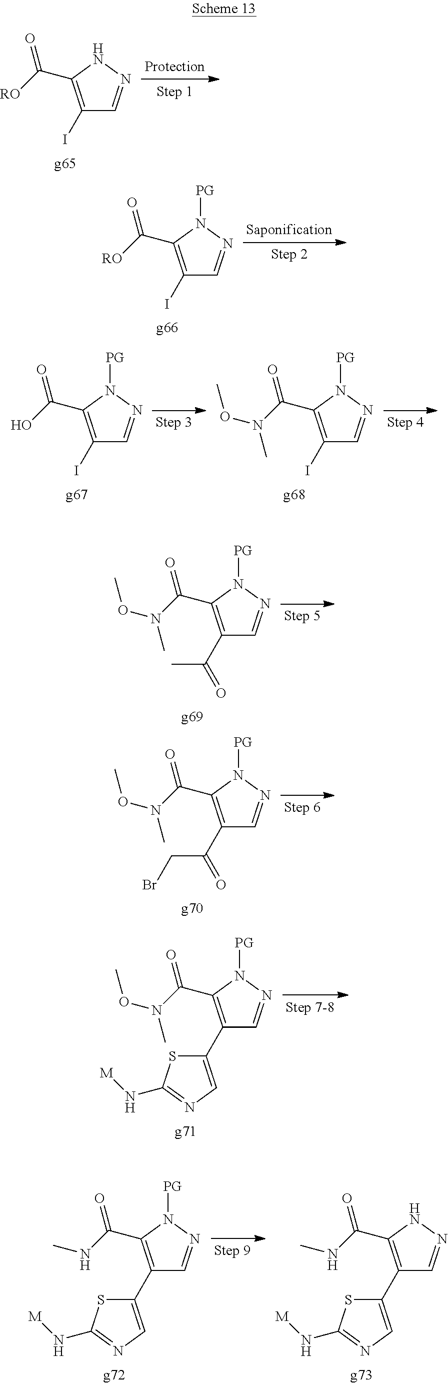

In one embodiment of the present invention, the compounds of Formula (I) may be prepared according to Scheme 14. Aminothiazole g9 can be protected, for example, by Boc₂O. The thiazole moiety of g74 was then brominated and subjected to Stille coupling to yield acetyl-substituted thiazole g76. Deprotection of g76 in the presence of HCl followed by Buchwald coupling allowed generation of aminothiazole g78. The final product g79 can be obtained by deprotection of the pyrazole in the presence of TFA and trifluoromethanesulfonic acid.

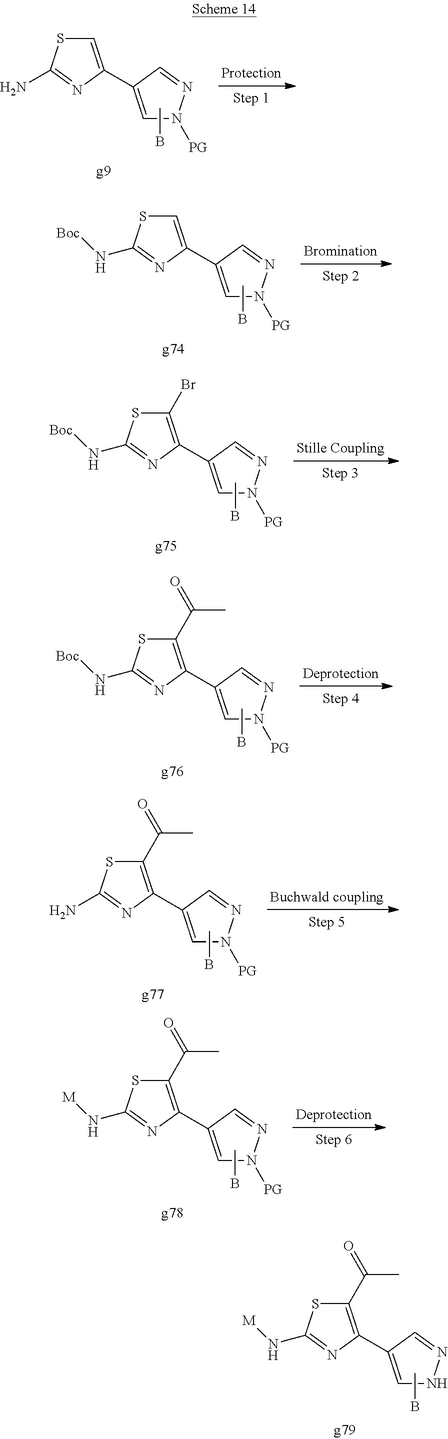

In one embodiment of the present invention, the compounds of Formula (I) may be prepared according to Scheme 15. Bromoketone g8 can undergo cyclisation with an appropriately substituted thiourea to afford thiazole g80. The thiazole moiety of g80 was then chlorinated and subjected to S_{N}Ar substitution in the presence of sodium ethoxide to yield ethoxy-substituted thiazole g82. Deprotection of g82 in the presence of TFA and trifluoromethanesulfonic acid finally afforded compound g83.

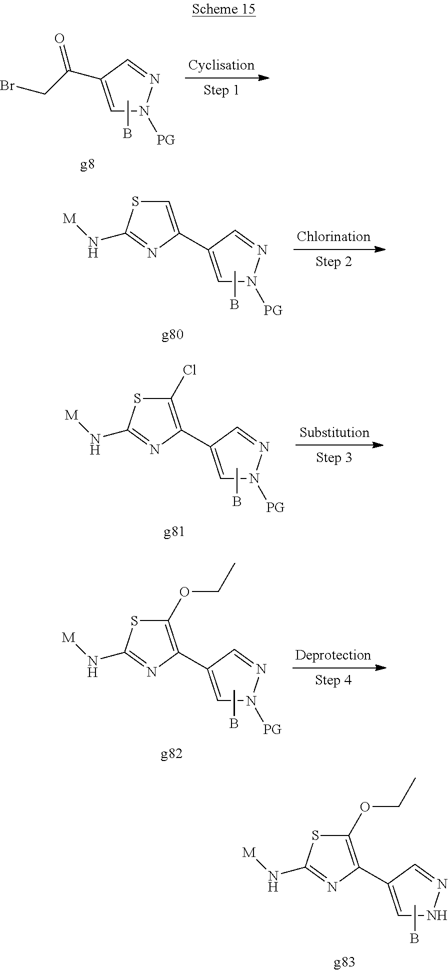

### EXPERIMENTAL

Unless otherwise noted, all starting materials were obtained from commercial suppliers and used without further purification.

Specifically, the following abbreviations may be used in the examples and throughout the specification.

| | |
|---|---|
| AcOH (Acetic acid) | mL (Milliliters) |
| BINAP (2,2′-bis(Diphenylphosphino)-1,1′- | μL (Microliters) |
| binaphthyl) | |
| Boc₂O (Di-tert-butyl dicarbonate) | mmol (Millimoles) |
| BuLi (Butyl lithium) | μmol (Micromoles) |
| tert-BuOK (Potassium tert-butoxide) | MOMCl (Methoxymethyl chloride) |
| tert-BuONO (tert-Butylnitrite) | M.p. (Melting point) |
| CHCl₃ (Chloroform) | NH₃ (Ammonia) |
| Cs₂CO₃ (Cesium carbonate) | NH₄Cl (Ammonium chloride) |
| CuBr₂ (Copper (II) bromide) | NMP (N-Methylpyrrolidone) |
| DAST (Diethylaminosulfur trifluoride) | NaBH₄ (Sodium borohydride) |
| DCM (Dichloromethane) | NaCl (Sodium chloride) |
| DMF (Dimethylformamide) | NaH (Sodium hydride) |
| EDCI•HCl (1-(3-dimethylaminopropyl)-3- | NaHCO₃ (Sodium hydrogenocarbonate) |
| ethylcarbodiimide) | |
| EtOAc (Ethyl acetate) | NaOH (Sodium hydroxide) |
| EtOH (Ethanol) | Na₂CO₃ (Sodium carbonate) |
| Et₂O (Diethyl ether) | Na₂SO₄ (Sodium sulphate) |
| Et₃N (Triethylamine) | PE (Petroleum ether) |
| h (Hour) | PdCl₂(dppf)(1,1′- |
| | Bis(diphenylphosphino)ferrocenepalladium(II) |
| | chloride) |
| HBr (Hydrobromic acid) | Pd(OAc)₂ (Palladium(II)acetate) |
| HCl (Hydrochloric acid) | Pd(PPh₃)₄ |
| | (Tetrakis(triphenylphosphine)palladium(0)) |
| KOAc (Potassium acetate) | Pd₂(dba)₃(Tris(dibenzylideneacetone)dipalladium(0)) |
| K₂CO₃ (Potassium carbonate) | PMBCl (p-Methoxybenzylchloride) |
| LCMS (Liquid Chromatography Mass | rt (Room temperature) |
| Spectrum) | |
| LDA (Lithium diisopropylamide) | RT (Retention Time) |
| M (Molar) | TBSCl (tert-Butyldimethylsilyl chloride |
| MeOH (Methanol) | TFA (Trifluoroacetic acid) |
| mg (Milligrams) | THF (Tetrahydrofuran) |
| MgSO₄ (Magnesium sulphate) | TMSCHN₂ (Trimethylsilyldiazomethane) |
| min (Minute) | UPLC-MS (Ultra Performance Liquid |
| | Chromatography Mass Spectrum) |
| min (Minute) | Xantphos (4,5-Bis(diphenylphosphino)-9,9- |
| | dimethylxanthene) |

All references to brine refer to a saturated aqueous solution of NaCl. Unless otherwise indicated, all temperatures are expressed in ° C. (degrees Centigrade). All reactions are conducted under an inert atmosphere at rt unless otherwise noted.

Most of the reactions were monitored by thin-layer chromatography on 0.25 mm Merck silica gel plates (60E-254), visualized with UV light. Flash column chromatography was performed on prepacked silica gel cartridges (15-40 μM, Merck).

Melting point determination was performed on a Buchi B-540 apparatus.

### LC-MS and UPLC-MS Methods:

### Method LC-MS

LC-MS were recorded on Agilent 1200 RRLC equipped with 6110 MSD with the following conditions: Reversed phase HPLC was carried out on Zorbax SB-C18 analytical column (5 μm, 2.1×50 mm) from Agilent, with a flow rate of 0.8 mL/min. The gradient conditions used are: 90% A (water+0.1% of trifluoroacetic acid), 10% B (acetonitrile+0.05% of trifluoroacetic acid) to 100% B at 3.5 minutes, kept till 4.0 minutes and equilibrated to initial conditions at 4.01 minutes until 4.5 minutes. Injection volume 2-5 μL. ES MS detector was used, acquiring in positive ionization mode.

### Method UPLC-MS

UPLC-MS were recorded on Waters ACQUITY UPLC with the following conditions: Reversed phase HPLC was carried out on BEH-C18 cartridge (1.7 μm, 2.1×50 mm) from Waters, with a flow rate of 0.8 mL/min. The gradient conditions used are: 90% A (water+0.1% of formic acid), 10% B (acetonitrile+0.1% of formic acid) to 100% B at 1.3 minutes, kept till 1.6 minutes and equilibrated to initial conditions at 1.7 minutes until 2.0 minutes. Injection volume 5 μL. ES MS detector was used, acquiring both in positive and negative ionization modes.

All mass spectra were taken under electrospray ionisation (ESI) methods.

Preparitive HPLC was conducted using a Gilson GX-281 preparative HPLC (322 Binary Gradient Module, 156 UV/Visible detector GX-281 injector/fraction collector) Phenomenex Synergi Max-Rp (C₁₂, 30×150 mm, 4 μm) or Kromasil Eternity (C₁₈, 30×150 mm, 5 μm) columns and H₂O+0.1% TFA and CH₃CN as eluents. Gradients used cover the range from 0% CH₃CN to 100% CH₃CN.

¹H-NMR spectra were recorded on a Bruker Avance 400 MHz or Varian 400 MHz spectrometer. Chemical shifts are expressed in parts per million (ppm, 6 units). Coupling constants are in units of hertz (Hz) Splitting patterns describe apparent multiplicities and are designated as s (singlet), d (doublet), t (triplet), q (quadruplet), m (multiplet), br (broad).

### EXAMPLES

### Example 1

### N-(3-Fluoro-6-methylpyridin-2-yl)-4-(3-(methoxymethyl)-1H-pyrazol-4-yl)thiazol-2-amine (Final Compound 1.36)

### Methyl 2-((dimethylamino)methylene)-4-methoxy-3-oxobutanoate

According to Scheme 1, Step 1: A solution of methyl 4-methoxy-3-oxobutanoate (8.21 mmol, 1.20 g) and of 1,1-dimethoxy-N,N-dimethylmethanamine (8.21 mmole, 1.09 mL) in DMF (12 mL) was heated in the microwave for 30 min at 120° C. After evaporation of the solvent, 1.61 g (7.98 mmol, 97%) of methyl 2-((dimethylamino)methylene)-4-methoxy-3-oxobutanoate as a brown oil was obtained and used without further purification.

UPLC-MS: RT=0.44 min; MS m/z ES⁺=202.

### Methyl 3-(methoxymethyl)-1H-pyrazole-4-carboxylate

According to Scheme 1, Step 2: A solution of methyl 2-((dimethylamino)methylene)-4-methoxy-3-oxobutanoate (7.98 mmol, 1.61 g), hydrazine hydrate (7.98 mmol, 0.39 mL) and AcOH (9.58 mmol, 0.55 mL) in butan-1-ol (25 mL) was stirred for 2 h, under reflux. After evaporation of the solvent, the resulting crude product was purified by flash chromatography over silica gel using DCM/MeOH (96:4) as eluent to yield methyl 3-(methoxymethyl)-1H-pyrazole-4-carboxylate (1.47 mmol, 0.25 g, 18%) as a yellow solid.

UPLC-MS: RT=0.45 min; MS m/z ES⁺=171.

### Methyl 1-(4-methoxybenzyl)-5-(methoxymethyl)-1H-pyrazole-4-carboxylate and methyl 1-(4-methoxybenzyl)-3-(methoxymethyl)-1H-pyrazole-4-carboxylate

According to Scheme 1, Step 3: 1-(Chloromethyl)-4-methoxybenzene (6.85 mmol, 0.93 mL) was added to a suspension of methyl 3-(methoxymethyl)-1H-pyrazole-4-carboxylate (6.23 mmol, 1.06 g) and K₂CO₃ (9.34 mmol, 1.29 g) in acetonitrile (40 mL) and then the reaction mixture was heated at 80° C. for 2.5 h. After evaporation of the solvent, water was added and the aqueous phase was extracted with EtOAc. The organic phase was dried over Na₂SO₄, was filtered and was concentrated under reduced pressure. Methyl 1-(4-methoxybenzyl)-5-(methoxymethyl)-1H-pyrazole-4-carboxylate and methyl 1-(4-methoxybenzyl)-3-(methoxymethyl)-1H-pyrazole-4-carboxylate (6.23 mmol, 1.81 g) were obtained as a yellow oil and the mixture of isomers was used without further purification.

UPLC-MS: RT=0.83 and 0.91 min; MS m/z ES⁺=291.

### 1-(4-Methoxybenzyl)-5-(methoxymethyl)-1H-pyrazole-4-carboxylic acid and 1-(4-methoxybenzyl)-3-(methoxymethyl)-1H-pyrazole-4-carboxylic acid

According to Scheme 1, Step 4: NaOH (18.6 mmol, 744 mg) was added to a solution of a mixture of methyl 1-(4-methoxybenzyl)-5-(methoxymethyl)-1H-pyrazole-4-carboxylate and methyl 1-(4-methoxybenzyl)-3-(methoxymethyl)-1H-pyrazole-4-carboxylate (6.20 mmol, 1.80 g) in water/MeOH (1:1, 30 mL) and the reaction mixture was heated at 80° C. for 1 h. After evaporation of the solvent, the residue was dissolved in water and the pH was adjusted to 1-2 with a solution of HCl 1 M. Then, the aqueous phase was extracted with DCM. The organic phase was dried over Na₂SO₄, was filtered and was concentrated under reduced pressure to yield a mixture of 1-(4-methoxybenzyl)-5-(methoxymethyl)-1H-pyrazole-4-carboxylic acid and 1-(4-methoxybenzyl)-3-(methoxymethyl)-1H-pyrazole-4-carboxylic acid (6.20 mmol, 1.71 g, 100%) as a beige solid. The crude product was used without any purification.

UPLC-MS: RT=0.68 and 0.73 min; MS m/z ES⁺=277.

### N-Methoxy-1-(4-methoxybenzyl)-5-(methoxymethyl)-N-methyl-M-pyrazole-4-carboxamide and N-methoxy-1-(4-methoxybenzyl)-3-(methoxymethyl)-N-methyl-1H-pyrazole-4-carboxamide

According to Scheme 1, Step 5: Oxalyl chloride (13.0 mmol, 1.10 mL) followed by a drop of DMF, were added to a solution of 1-(4-methoxybenzyl)-5-(methoxymethyl)-1H-pyrazole-4-carboxylic acid and 1-(4-methoxybenzyl)-3-(methoxymethyl)-1H-pyrazole-4-carboxylic acid (6.20 mmol, 1.71 g) in DCM (30 mL), at 0° C., and the reaction mixture was stirred for 30 min at rt. After evaporation, the crude product was dissolved in DCM (30 mL) and a solution of N,O-dimethylhydroxylamine hydrochloride (9.77 mmol, 0.95 g) and Et₃N (16.3 mmol, 2.27 mL) in DCM (30 mL) was added at 0° C. The reaction mixture was stirred for 1 h at rt. Then the reaction was quenched with water and the aqueous phase was extracted with DCM. The organic phase was dried over Na₂SO₄, was filtered and was concentrated. The resulting crude product was purified by flash chromatography over silica gel using DCM/MeOH (97:3) as eluent to yield N-methoxy-1-(4-methoxybenzyl)-5-(methoxymethyl)-N-methyl-1H-pyrazole-4-carboxamide and N-methoxy-1-(4-methoxybenzyl)-3-(methoxymethyl)-N-methyl-1H-pyrazole-4-carboxamide (5.10 mmol, 1.63 g, 82%) as an orange oil.

UPLC-MS: RT=0.74 and 0.80 min; MS m/z ES⁺=320.

### 1-O-(4-Methoxybenzyl)-5-(methoxymethyl)-1H-pyrazol-4-yl)ethanone and 1-(1-(4-methoxybenzyl)-3-(methoxymethyl)-1H-pyrazol-4-yl)ethanone

According to Scheme 1, Step 6: Methylmagnesium bromide (3 M, 1.03 mmol, 0.34 mL) was added, at 0° C., to a solution of N-methoxy-1-(4-methoxybenzyl)-5-(methoxymethyl)-N-methyl-1H-pyrazole-4-carboxamide and N-methoxy-1-(4-methoxybenzyl)-3-(methoxymethyl)-N-methyl-1H-pyrazole-4-carboxamide (0.34 mmol, 110 mg) in THF (3 mL) and the reaction mixture was stirred for 2 h at rt. The reaction was quenched with a solution of HCl (1 M) and the aqueous phase was extracted with DCM. The organic phase was dried over Na₂SO₄, was filtered and was concentrated to yield 1-(1-(4-methoxybenzyl)-5-(methoxymethyl)-1H-pyrazol-4-yl)ethanone and 1-(1-(4-methoxybenzyl)-3-(methoxymethyl)-1H-pyrazol-4-yl)ethanone (0.34 mmol, 94 mg) as a colorless oil. The mixture was used without further purification.

UPLC-MS: RT=0.75 and 0.83 min; MS m/z ES⁺=275.

### 2-Bromo-1-(1-(4-methoxybenzyl)-5-(methoxymethyl)-1,1-pyrazol-4-yl)ethanone and 2-bromo-1-O-(4-methoxybenzyl)-3-(methoxymethyl)-1H-pyrazol-4-yl)ethanone

According to Scheme 1, Step 7: A solution of yield 1-(1-(4-methoxybenzyl)-5-(methoxymethyl)-1H-pyrazol-4-yl)ethanone and 1-(1-(4-methoxybenzyl)-3-(methoxymethyl)-1H-pyrazol-4-yl)ethanone (4.74 mmol, 1.30 g) and CuBr₂ (9.48 mmol, 2.12 g) in MeOH (30 mL) was stirred under reflux for 2 h. After evaporation of the solvent, the reaction was diluted with water and the aqueous phase was extracted with DCM. The organic phase was dried over Na₂SO₄, was filtered and was concentrated to yield 2-bromo-1-(1-(4-methoxybenzyl)-5-(methoxymethyl)-1H-pyrazol-4-yl)ethanone and 2-bromo-1-(1-(4-methoxybenzyl)-3-(methoxymethyl)-1H-pyrazol-4-yl)ethanone (4.74 mmol, 1.68 g) as a brown oil.

UPLC-MS: RT=0.88 and 0.95 min; MS m/z ES⁺=353, 355.

### 4-(1-(4-Methoxybenzyl)-5-(methoxymethyl)-1H-pyrazol-4-yl)thiazol-2-amine and 4-(1-(4-methoxybenzyl)-3-(methoxymethyl)-1H-pyrazol-4-yl)thiazol-2-amine

According to Scheme 1, Step 8: A solution of a mixture of 2-bromo-1-(1-(4-methoxybenzyl)-5-(methoxymethyl)-1H-pyrazol-4-yl)ethanone and 2-bromo-1-(1-(4-methoxybenzyl)-3-(methoxymethyl)-1H-pyrazol-4-yl)ethanone (1.70 mmol, 600 mg) and of thiourea (1.70 mmol, 129 mg) in acetone (10 mL) was stirred at 45° C. overnight. After evaporation of the solvent, 4-(1-(4-methoxybenzyl)-5-(methoxymethyl)-1H-pyrazol-4-yl)thiazol-2-amine and 4-(1-(4-methoxybenzyl)-3-(methoxymethyl)-1H-pyrazol-4-yl)thiazol-2-amine (1.70 mmol, 561 mg) were obtained as a brown solid.

UPLC-MS: RT=0.62 min; MS m/z ES⁺=331.

### N-(3-Fluoro-6-methylpyridin-2-yl)-4-O-(4-methoxybenzyl)-5-(methoxymethyl)-1H-pyrazol-4-yl)thiazol-2-amine and N-(3-fluoro-6-methylpyridin-2-yl)-4-(1-(4-methoxybenzyl)-3-(methoxymethyl)-1H-pyrazol-4-yl)thiazol-2-amine

According to Scheme 1, Step 9: A solution of 4-(1-(4-methoxybenzyl)-5-(methoxymethyl)-1H-pyrazol-4-yl)thiazol-2-amine and 4-(1-(4-methoxybenzyl)-3-(methoxymethyl)-1H-pyrazol-4-yl)thiazol-2-amine (0.45 mmol, 150 mg), 2-bromo-3-fluoro-6-methylpyridine (0.54 mmol, 104 mg), Xantphos (68 μmol, 39 mg), Pd(OAc)₂ (45 μmol, 10 mg) and cesium carbonate (0.90 mmol, 296 mg) in dioxane (1 mL) was heated in the microwave at 135° C. for 45 min. After filtration through celite and evaporation of the solvent, the resulting crude product was purified by flash chromatography over silica gel using cyclohexane/EtOAc (100:0 to 70:30) as eluent to afford N-(3-fluoro-6-methylpyridin-2-yl)-4-(1-(4-methoxybenzyl)-5-(methoxymethyl)-1H-pyrazol-4-yl)thiazol-2-amine and N-(3-fluoro-6-methylpyridin-2-yl)-4-(1-(4-methoxybenzyl)-3-(methoxymethyl)-1H-pyrazol-4-yl)thiazol-2-amine (0.19 mmol, 84 mg, 40%).

UPLC-MS: RT=1.15 and 1.19 min; MS m/z ES⁺=440.

### N-(3-Fluoro-6-methylpyridin-2-yl)-4-(3-(methoxymethyl)-1H-pyrazol-4-yl)thiazol-2-amine

According to Scheme 1, Step 10: A solution of a mixture of N-(3-fluoro-6-methylpyridin-2-yl)-4-(1-(4-methoxybenzyl)-5-(methoxymethyl)-1H-pyrazol-4-yl)thiazol-2-amine and N-(3-fluoro-6-methylpyridin-2-yl)-4-(1-(4-methoxybenzyl)-3-(methoxymethyl)-1H-pyrazol-4-yl)thiazol-2-amine (0.19 mmol, 84 mg) in TFA (3 mL) was heated in the microwave for 10 min at 80° C. The crude residue was neutralized with a saturated solution of Na₂CO₃ and the aqueous phase was extracted with EtOAc. The organic phase was dried over MgSO₄, was filtered and was concentrated. The resulting mixture was purified by flash chromatography over silica gel using DCM/DEN (100:0 to 70:30; DEN: DCM/EtOH/NH₃ 90:9:1) as eluent to yield after evaporation N-(3-fluoro-6-methylpyridin-2-yl)-4-(3-(methoxymethyl)-1H-pyrazol-4-yl)thiazol-2-amine (13 μmol, 4.2 mg, 7%) as a yellow solid.

UPLC-MS: RT=0.88 min; MS m/z ES⁺=320.

### Example 2

### 4-(5-(1-Methoxyethyl)-1H-pyrazol-4-yl)-N-(4-methylpyrimidin-2-yl)thiazol-2-amine (Final Compound 1-29)

### Ethyl 3-(1-methoxyethyl)-1-(methoxymethyl)-1H-pyrazole-4-carboxylate

According to Scheme 2, Step 1: K₂CO₃ (9.08 mmol, 1.25 g) and chloro(methoxy)methane (9.08 mmol, 0.69 mL) were added to a solution of ethyl 3-(1-methoxyethyl)-1H-pyrazole-4-carboxylate (6.05 mmol, 1.20 g) in acetonitrile (40 mL) and then the reaction mixture was heated at 40° C. for 2 h. After evaporation of the solvent, water was added and the aqueous phase was extracted with DCM. The organic phase was dried over MgSO₄, was filtered and was concentrated under reduced pressure. The resulting mixture was purified by flash chromatography over silica gel using DCM/MeOH (100:0 to 95:5) as eluent to yield after evaporation ethyl 341-methoxyethyl)-1-(methoxymethyl)-1H-pyrazole-4-carboxylate (3.30 mmol, 54%) as a yellow oil and was used without further purification.

UPLC-MS: RT=0.87 min; MS m/z ES⁺=243.

### N-Methoxy-3-(1-methoxyethyl)-1-(methoxymethyl)-N-methyl-M-pyrazole-4-carboxamide

According to Scheme 2, Step 2: Trimethylaluminium (9.91 mmol, 4.95 mL) was added dropwise at 0° C., to a solution of ethyl 3-(1-methoxyethyl)-1-(methoxymethyl)-1H-pyrazole-4-carboxylate (1.24 mmol, 300 mg) in DCM (2 mL). The reaction mixture was stirred for 20 min at 0° C. and for another 20 min at rt. After cooling the reaction mixture to 0° C., a solution of N,O-dimethylhydroxylamine hydrochloride (4.95 mmol, 483 mg) was added and the solution was stirred under reflux for 2 h. Then the reaction was quenched with water and the aqueous phase was extracted with DCM. The organic phase was dried over MgSO₄, was filtered and was concentrated under reduced pressure. The resulting mixture was purified by flash chromatography over silica gel using DCM/MeOH (100:0 to 98:2) as eluent to yield after evaporation N-methoxy-3-(1-methoxyethyl)-1-(methoxymethyl)-N-methyl-1H-pyrazole-4-carboxamide (0.39 mmol, 100 mg, 31%).

UPLC-MS: RT=0.52 min; MS m/z ES⁺=258.

### 1-(3-(1-Methoxyethyl)-1-(methoxymethyl)-1H-pyrazol-4-Aethanone

According to Scheme 2, Step 3: Methylmagnesium bromide (3 M, 1.17 mmol, 0.39 mL) was added dropwise at 0° C. to a solution of N-methoxy-3-(1-methoxyethyl)-1-(methoxymethyl)-N-methyl-1H-pyrazole-4-carboxamide (0.39 mmol, 100 mg) in THF (2 mL) and the reaction mixture was stirred for 1 h. The reaction was quenched with water and the aqueous phase was extracted with EtOAc. The organic phase was dried over MgSO₄, was filtered and was concentrated to yield 1-(3-(1-methoxyethyl)-1-(methoxymethyl)-1H-pyrazol-4-yl)ethanone (0.33 mmol, 70 mg, 85%) and was used without further purification.

UPLC-MS: RT=0.52 min; MS m/z ES⁺=213.

### 2-Bromo-1-(3-(1-methoxyethyl)-1H-pyrazol-4-yl)ethanone

According to Scheme 2, Step 4: A solution of 1-(3-(1-methoxyethyl)-1-(methoxymethyl)-1H-pyrazol-4-yl)ethanone (0.33 mmol, 70 mg) and phenyltrimethylammonium tribromide (0.33 mmol, 124 mg) in CHCl₃ (2 mL) was stirred at rt for 1 h. The reaction was quenched with water and the aqueous phase was extracted with EtOAc. The organic phase was dried over MgSO₄, was filtered and was concentrated to yield 2-bromo-1-(3-(1-methoxyethyl)-1H-pyrazol-4-yl)ethanone (0.12 mmol, 30 mg, 28%) and was used without further purification.

UPLC-MS: RT=0.71 min; MS m/z ES⁺=291, 293.

### 4-(5-(1-Methoxyethyl)-1H-pyrazol-4-yl)-N-(4-methylpyrimidin-2-yl)thiazol-2-amine

According to Scheme 2, Step 5: A solution of 2-bromo-1-(3-(1-methoxyethyl)-1H-pyrazol-4-yl)ethanone (0.12 mmol, 30 mg) and of 1-(4-methylpyrimidin-2-yl)thiourea (0.12 mmol, 20.4 mg) in EtOH (3 mL) was stirred at 80° C. for 30 min. Then the reaction was quenched with water and the aqueous phase was extracted with EtOAc. The organic phase was dried over MgSO₄, was filtered and was concentrated under reduced pressure. The resulting mixture was purified by flash chromatography over silica gel using DCM/MeOH (100:0 to 95:5) as eluent to yield after evaporation 4-(5-(1-methoxyethyl)-1H-pyrazol-4-yl)-N-(4-methylpyrimidin-2-yl)thiazol-2-amine (8.2 μmol, 2.6 mg, 7%) as a beige solid.

UPLC-MS: RT=0.75 min; MS m/z ES⁺=317.

### Example 3

### 2-(5-Methyl-1,2,4-thiadiazol-3-ylamino)-4-(1H-pyrazol-4-yl) thiazole-5-carbonitrile (Final Compound 1-44)

### Ethyl 1-(4-methoxybenzyl)-1H-pyrazole-4-carboxylate

According to Scheme 3, Step 1: 1-(Chloromethyl)-4-methoxybenzene (157 mmol, 24.5 g) and K₂CO₃ (39.43 g, 285.71 mmol) were added to a solution of ethyl 1H-pyrazole-4-carboxylate (143 mmol, 20.0 g) in acetonitrile (150 mL). The resulting mixture was stirred under reflux for 5 h. After cooling to rt, the mixture was filtered and concentrated in vacuum. The residue was purified by flash chromatography over silica gel using EtOAc/PE (1:20 to 1:5) as eluent to afford ethyl 1-(4-methoxybenzyl)-1H-pyrazole-4-carboxylate (126.7 mmol, 33 g, 89%) as an off-white solid.

LC-MS: m/z ES⁺=261.

### 3-(1-(4-Methoxybenzyl)-1H-pyrazol-4-yl)-3-oxopropanenitrile

According to Scheme 3, Step 2: At −78° C., BuLi (23 mmol, 9.2 mL, 2.5M) was added dropwise to a solution of acetonitrile (21.1 mmol, 0.87 g) in THF (25 mL). The resulting mixture was stirred at this temperature for 20 min and then ethyl 1-(4-methoxybenzyl)-1H-pyrazole-4-carboxylate (19.2 mmol, 5.00 g) in THF (25 mL) was added. The reaction mixture was stirred at −78° C. for 1 h and then allowed to warm up to rt and stirred for another 1 h. The reaction was quenched with saturated NH₄Cl aqueous solution and extracted with EtOAc (50 mL×3). The combined organic phases were dried over MgSO₄, filtered and concentrated under reduced pressure. The residue was purified on combi-flash EtOAc/PE (1:15 to 1:5) to give 3-(1-(4-methoxybenzyl)-1H-pyrazol-4-yl)-3-oxopropanenitrile (13.1 mmol, 3.33 g, 68%).

¹H-NMR (DMSO-d₆, 400 MHz): δ 8.62 (s, 1H), 8.05 (s, 1H), 7.32 (s, 1H), 6.97 (d, J=7.0 Hz, 2H), 5.36 (s, 1H), 4.51 (s, 1H), 3.79 (s, 1H).

### 2-Bromo-3-(1-(4-methoxybenzyl)-1H-pyrazol-4-yl)-3-oxopropanenitrile

According to Scheme 3, Step 3: A suspension of 3-(1-(4-methoxybenzyl)-1H-pyrazol-4-yl)-3-oxopropanenitrile (33.7 mmol, 8.60 g) and CuBr₂ (67.4 mmol, 15.0 g) in a mixture of THF/EtOAc/CHCl₃ (150 mL/20 mL/20 mL) was stirred at reflux for 3 h. After cooling to rt, the mixture was filtered and the green filtrate was washed with water. The organic phase was dried over MgSO₄, filtered and concentrated under reduced pressure. The residue was purified on combi-flash EtOAc/PE (1:15 to 1:5) to give 2-bromo-3-(1-(4-methoxybenzyl)-1H-pyrazol-4-yl)-3-oxopropanenitrile (25.3 mmol, 8.44 g, 75%).

LC-MS: m/z ES⁺=334, 336.

### 2-Amino-4-(1-(4-methoxybenzyl)-1H-pyrazol-4-Athiazole-5-carbonitrile

According to Scheme 3, Step 4: To a solution of 2-bromo-3-(1-(4-methoxybenzyl)-1H-pyrazol-4-yl)-3-oxopropanenitrile (25.3 mmol, 8.44 g) in EtOH (120 mL) was added thiourea (26.5 mmol, 2.06 g). The resulting mixture was stirred at reflux for 2 h. After cooling to rt, the solvent was removed under reduced pressure. The residue was purified by silica column on combi-flash EtOAc/PE (1:10 to ˜1:1) to give 2-amino-4-(1-(4-methoxybenzyl)-1H-pyrazol-4-yl)thiazole-5-carbonitrile as a yellow solid (22.0 mmol, 6.83 g, 87%).

LC-MS: m/z ES⁺=312.

### 2-Bromo-4-(1-(4-methoxybenzyl)-1H-pyrazol-4-yl)thiazole-5-carbonitrile

According to Scheme 3, Step 5: To a solution of 2-amino-4-(1-(4-methoxybenzyl)-1H-pyrazol-4-yl)thiazole-5-carbonitrile (33.7 mmol, 10.5 g) in acetonitrile (100 mL) was added CuBr₂ (37.1 mmol, 8.28 g) and tert-BuONO (40.5 mmol, 4.17 g). The resulting mixture was stirred at rt for 30 min under nitrogen atmosphere and then stirred at 70-80° C. for another 1 h. After cooling to rt, the mixture was filtered and concentrated under reduced pressure. The residue was purified by silica column on combi-flash EtOAc/PE (1:10 to 1:1) to give 2-bromo-4-(1-(4-methoxybenzyl)-1H-pyrazol-4-yl)thiazole-5-carbonitrile (26.6 mmol, 9.99 g, 79%) as a brown solid.

LC-MS: m/z ES⁺=375, 377.

### 4-(1-(4-Methoxybenzyl)-1H-pyrazol-4-yl)-2-(5-methyl-1,2,4-thiadiazol-3-ylamino) thiazole-5-carbonitrile

According to Scheme 3, Step 6: To a solution of 2-bromo-4-(1-(4-methoxybenzyl)-1H-pyrazol-4-yl)thiazole-5-carbonitrile (1.09 mmol, 408 mg) and 5-methyl-1,2,4-thiadiazol-3-amine (0.88 mmol, 100 mg) in dioxane (6 mL) was added Pd₂(dba)₃ (0.04 mmol, 37 mg), Xantphos (0.08 mmol, 46 mg) and K₂CO₃ (2.40 mmol, 332 mg). The resulting mixture was stirred at reflux under nitrogen overnight. After cooling to rt, the mixture was filtered and concentrated under reduced pressure. The residue was purified by preparative TLC PE/EtOAc (1:1) to give 4-(1-(4-methoxybenzyl)-1H-pyrazol-4-yl)-2-(5-methyl-1,2,4-thiadiazol-3-ylamino)thiazole-5-carbonitrile as a white solid (0.25 mmol, 92 mg, 28%).

LC-MS: m/z ES⁺=410.

### 2-(5-Methyl-1,2,4-thiadiazol-3-ylamino)-4-(1H-pyrazol-4-yl)thiazole-5-carbonitrile

According to Scheme 3, Step 7: A solution of 4-(1-(4-methoxybenzyl)-1H-pyrazol-4-yl)-2-(5-methyl-1,2,4-thiadiazol-3-ylamino)thiazole-5-carbonitrile (0.22 mmol, 90 mg) in TFA (5 mL) was stirred at 100° C. for 10 min under microwave conditions. After cooling to rt, the solvent was removed under reduced pressure. The residue was diluted with EtOAc and washed with saturated aqueous NaHCO₃ solution. The organic phase was dried over MgSO₄, filtered and concentrated under reduced pressure. The crude product was purified by preparative HPLC to give 2-(5-methyl-1,2,4-thiadiazol-3-ylamino)-4-(1H-pyrazol-4-yl)thiazole-5-carbonitrile as a white solid (97 μmol, 28 mg, 44%).

LC-MS: RT=2.62 min; m/z ES⁺=290.

¹H-NMR (DMSO-d₆, 400 MHz): δ 13.38 (s, 1H), 13.22 (s, 1H), 8.26 (s, 1H), 8.02 (s, 1H), 2.79 (s, 3H).

### Example 4

### 4-(3-(Difluoromethyl)-1H-pyrazol-4-yl)-N-(4-methylpyrimidin-2-yl) thiazol-2-amine (Final Compound 1-1)

### 3-Formyl-N-methoxy-1-(4-methoxybenzyl)-N-methyl-1H-pyrazole-4-carboxamide

According to Scheme 4, Step 1: BuLi 2.5 M (14.5 mmol, 5.81 mL) was added to a solution of diisopropylamine (14.5 mmol, 1.47 g) in THF (10 mL) at −78° C. and the reaction mixture was stirred at 0° C. for 25 min. The resulting LDA solution was added at −78° C. to a solution of N-methoxy-1-(4-methoxybenzyl)-N-methyl-1H-pyrazole-4-carboxamide (7.26 mmol, 2.00 g) in THF (5 mL) and the reaction mixture was stirred for 5 min at −78° C. Then DMF (72.6 mmol, 5.31 g) was added to the reaction mixture at −78° C. and the solution was stirred for 5 min at −78° C. and for 1 h at rt. The reaction mixture was quenched with water (50 mL) and the aqueous phase was extracted with EtOAc. The organic phase was dried over MgSO₄, was filtered and was concentrated to yield a brown oil. The resulting crude product was purified by flash chromatography over silica gel using cyclohexane/EtOAc (100:0 to 50:50) as eluent to yield after evaporation 3-formyl-N-methoxy-1-(4-methoxybenzyl)-N-methyl-1H-pyrazole-4-carboxamide (4.94 mmol, 1.50 g, 68%) as a beige solid.

UPLC-MS: RT=0.85 min; MS m/z ES⁺=304.

### 3-(Difluoromethyl)-N-methoxy-1-(4-methoxybenzyl)-N-methyl-1H-pyrazole-4-carboxamide

According to Scheme 4, Step 2: DAST (1.48 mmol, 196 μL) was added to a solution of 3-formyl-N-methoxy-1-(4-methoxybenzyl)-N-methyl-1H-pyrazole-4-carboxamide (0.99 mmol, 300 mg) in DCM (2 mL) at 0° C. and the reaction mixture was stirred at rt for 6 h. The reaction mixture was quenched with a saturated solution of NaHCO₃ and the aqueous phase was extracted with DCM. The organic phase was dried over MgSO₄, was filtered and was concentrated to afford 3-(difluoromethyl)-N-methoxy-1-(4-methoxybenzyl)-N-methyl-1H-pyrazole-4-carboxamide (0.61 mmol, 200 mg, 62%) which was used without any purification.

UPLC-MS: RT=0.94 min; MS m/z ES⁺=326.

### 1-(3-(Difluoromethyl)-1-(4-methoxybenzyl)-1H-pyrazol-4-yl)ethanone

According to Scheme 4, Step 3: The compound was synthesized with the same procedure as used in Example 2, Step 3.

UPLC-MS: RT=0.96 min; MS m/z ES⁺=282.

### 2-Bromo-1-(3-(difluoromethyl)-1-(4-methoxybenzyl)-1H-pyrazol-4-yl)ethanone

According to Scheme 4, Step 4: Trimethylphenylammonium tribromide (0.61 mmol, 228 mg) was added to a solution of 1-(3-(difluoromethyl)-1-(4-methoxybenzyl)-1H-pyrazol-4-yl)ethanone (0.61 mmol, 170 mg) in CHCl₃ (2 mL) at 0° C. and the reaction mixture was stirred at 40° C. for 1 h. The reaction mixture was quenched with water and the aqueous phase was extracted with DCM. The organic phase was dried over MgSO₄, was filtered and was concentrated to afford 2-bromo-1-(3-(difluoromethyl)-1-(4-methoxybenzyl)-1H-pyrazol-4-yl)ethanone (0.56 mmol, 200 mg, 92%) which was used without any purification.

UPLC-MS: RT=1.05 min; MS m/z ES⁺=359, 361.

### 4-(3-(Difluoromethyl)-1-(4-methoxybenzyl)-1H-pyrazol-4-yl)-N-(4-methylpyrimidin-2-yl)thiazol-2-amine

According to Scheme 4, Step 5: A solution of 2-bromo-1-(3-(difluoromethyl)-1-(4-methoxybenzyl)-1H-pyrazol-4-yl)ethanone (1.19 mmol, 427 mg) and of 1-(4-methylpyrimidin-2-yl)thiourea (1.19 mmol, 0.200 mg) in EtOH (2 mL) was stirred at 80° C. for 30 min. Then the reaction mixture was filtered and the resulting filtrate was concentrated. The resulting mixture was purified by flash chromatography over silica gel using DCM/DEN (100:0 to 60:40; DEN: DCM/EtOH/NH₃ 90:9:1) as eluent to yield after evaporation 4-(3-(difluoromethyl)-1-(4-methoxybenzyl)-1H-pyrazol-4-yl)-N-(4-methylpyrimidin-2-yl)thiazol-2-amine (0.72 mmol, 310 mg, 61%).

UPLC-MS: RT=1.17 min; MS m/z ES⁺=429.

### 4-(3-(Difluoromethyl)-1H-pyrazol-4-yl)-N-(4-methylpyrimidin-2-yl)thiazol-2-amine

According to Scheme 4, Step 6: A solution of 4-(3-(difluoromethyl)-1-(4-methoxybenzyl)-1H-pyrazol-4-yl)-N-(4-methylpyrimidin-2-yl)thiazol-2-amine (0.72 mmol, 310 mg) in TFA (2 mL) was heated in the microwave for 5 min at 80° C. After evaporation of the solvent, the resulting residue was neutralized with a saturated solution of Na₂CO₃ and the aqueous phase was extracted with EtOAc. The organic phase was dried over MgSO₄, was filtered and was concentrated. The resulting crude product was purified by flash chromatography over silica gel using DCM/DEN (100:0 to 60:40; DEN: DCM/EtOH/NH₃ 90:9:1) as eluent to yield after evaporation 4-(3-(difluoromethyl)-1H-pyrazol-4-yl)-N-(4-methylpyrimidin-2-yl)thiazol-2-amine (0.26 mmol, 18 mg, 36%) as an orange solid.

M.p.: 241-242° C.;

UPLC-MS: RT=0.82 min; MS m/z ES⁺=309.

### Example 5

### 4-(5-(Cyclobutoxymethyl)-1H-pyrazol-4-yl)-N-(4-methylpyrimidin-2-yl)thiazol-2-amine (Final Compound 1-28)

### Ethyl 3-formyl-1-(methoxymethyl)-1H-pyrazole-4-carboxylate and ethyl 5-formyl-1-(methoxymethyl)-1H-pyrazole-4-carboxylate

According to Scheme 5, Step 1: BuLi 2.5 M (1.63 mmol, 0.81 mL) was added to a solution of diisopropylamine (1.63 mmol, 0.23 mL) in THF (10 mL) at −78° C. and the reaction mixture was stirred at 0° C. for 25 min. The resulting LDA solution was added at −78° C. to a solution of ethyl 1-(methoxymethyl)-1H-pyrazole-4-carboxylate (1.09 mmol, 200 mg) in THF (5 mL) and the reaction mixture was stirred for 5 min at −78° C. Then DMF (8.69 mmol, 0.67 mL) was added to the reaction mixture at −78° C. and the solution was stirred for 5 min. The reaction mixture was quenched with a saturated solution of NH₄Cl and the aqueous phase was extracted with DCM. The organic phase was dried over MgSO₄, was filtered and was concentrated to yield a mixture of ethyl 3-formyl-1-(methoxymethyl)-1H-pyrazole-4-carboxylate and ethyl 5-formyl-1-(methoxymethyl)-1H-pyrazole-4-carboxylate (0.80 mmol, 170 mg, 74%) which was used without any purification.

UPLC-MS: RT=0.66, 0.75 min; MS m/z ES⁺=213.

### Ethyl 3-(hydroxymethyl)-1-(methoxymethyl)-1H-pyrazole-4-carboxylate

According to Scheme 5, Step 2: NaBH₄ (0.80 mmol, 30 mg) was added to a solution of ethyl 3-formyl-1-(methoxymethyl)-1H-pyrazole-4-carboxylate and ethyl 5-formyl-1-(methoxymethyl)-1H-pyrazole-4-carboxylate (0.80 mmol, 170 mg) in EtOH (3 mL), at 0° C., and the reaction mixture was stirred for 1 h at rt. The reaction mixture was quenched with water. The aqueous phase was extracted with butan-2-ol and the organic phase was dried over MgSO₄, was filtered and was concentrated to yield ethyl 3-(hydroxymethyl)-1-(methoxymethyl)-1H-pyrazole-4-carboxylate (0.47 mmol, 100 mg, 58%) which was used without any purification.

UPLC-MS: RT=0.55 min; MS m/z ES⁺=215.

### Ethyl 1-(methoxymethyl)-3-((phenylsulfonyloxy)methyl)-1H-pyrazole-4-carboxylate

According to Scheme 5, Step 3: To a solution of ethyl 3-(hydroxymethyl)-1-(methoxymethyl)-1H-pyrazole-4-carboxylate (4.43 mmol, 950 mg) in THF (2 mL), was added Et₃N (5.32 mmol, 0.74 mL). After cooling the reaction mixture at 0° C., benzene sulfonyl chloride (5.32 mmol, 0.68 mL) was added and the reaction mixture was stirred at rt for 2 h. As the reaction was not complete, Et₃N (5.32 mmol, 0.74 mL) and benzene sulfonyl chloride (5.32 mmol, 0.68 mL) were added and the reaction mixture was stirred at rt for 1 h. Then the reaction mixture was quenched with a saturated solution of NaHCO₃ and the aqueous phase was extracted with EtOAc. The organic phase was dried over MgSO₄, was filtered and was concentrated to yield ethyl 1-(methoxymethyl)-3-((phenyl sulfonyloxy)methyl)-1H-pyrazole-4-carboxylate (3.85 mmol, 1.36 g, 87%) which was used without any purification.

UPLC-MS: RT=0.96 min; MS m/z ES⁺=355.

### Ethyl 3-(cyclobutoxymethyl)-1-(methoxymethyl)-1H-pyrazole-4-carboxylate

According to Scheme 5, Step 4: At 0° C., NaH (0.31 mmol, 12 mg) was added to a solution of cyclobutanol (0.31 mmol, 24 μL) in THF (1 mL) and the reaction mixture was stirred at rt for 30 min. Then a solution of ethyl 1-(methoxymethyl)-3-((phenylsulfonyloxy)methyl)-1H-pyrazole-4-carboxylate (0.21 mmol, 73 mg) in THF (1 mL) was added and the reaction mixture was stirred overnight at rt. The reaction mixture was quenched with water and the aqueous phase was extracted with EtOAc. The organic phase was dried over MgSO₄, was filtered and was concentrated to yield ethyl 3-(cyclobutoxymethyl)-1-(methoxymethyl)-1H-pyrazole-4-carboxylate (78 μmol, 35 mg, 38%) which was used without any purification.

UPLC-MS: RT=0.97 min; MS m/z ES⁺=269.

### 3-(Cyclobutoxymethyl)-N-methoxy-1-(methoxymethyl)-N-methyl-1H-pyrazole-4-carboxamide

According to Scheme 5, Step 5: The compound was synthesized with the same procedure as used in Example 2, Step 2 to yield 3-(cyclobutoxymethyl)-N-methoxy-1-(methoxymethyl)-N-methyl-1H-pyrazole-4-carboxamide (0.11 mmol, 40 mg, 87%).

UPLC-MS: RT=0.75 min; MS m/z ES⁺=284.

### 1-(3-(Cyclobutoxymethyl)-1-(methoxymethyl)-1H-pyrazol-4-yl)ethanone

According to Scheme 5, Step 6: The compound was synthesized with the same procedure as used in Example 2, Step 3 to yield 1-(3-(cyclobutoxymethyl)-1-(methoxymethyl)-1H-pyrazol-4-yl)ethanone (0.13 mmol, 31 mg, 92%).

UPLC-MS: RT=0.78 min; MS m/z ES⁺=239.

### 2-Bromo-1-(3-(cyclobutoxymethyl)-1-(methoxymethyl)-1H-pyrazol-4-yl)ethanone

According to Scheme 5, Step 7: The compound was synthesized with the same procedure as used in Example 4, Step 4 to yield 2-bromo-1-(3-(cyclobutoxymethyl)-1-(methoxymethyl)-1H-pyrazol-4-yl)ethanone (0.11 mmol, 30 mg, 42%) and with the presence of 2,2-dibromo-1-(3-(cyclobutoxymethyl)-1-(methoxymethyl)-1H-pyrazol-4-yl)ethanone (85 pmol, 30 mg, 33%).

UPLC-MS: RT=0.75 min; MS m/z ES⁺=273.

### 4-(5-(Cyclobutoxymethyl)-1H-pyrazol-4-yl)-N-(4-methylpyrimidin-2-yl)thiazol-2-amine

According to Scheme 5, Step 8: A solution of 2-bromo-1-(3-(cyclobutoxymethyl)-1-(methoxymethyl)-1H-pyrazol-4-yl)ethanone (0.11 mmol, 30 mg) and of 1-(4-methylpyrimidin-2-yl)thiourea (0.11 mmol, 18.5 mg) in EtOH (3 mL) was stirred at 80° C. for 30 min. Then the reaction mixture was filtered and the filtrate was concentrated. The crude residue was purified by preparative-HPLC to yield 4-(5-(cyclobutoxymethyl)-1H-pyrazol-4-yl)-N-(4-methylpyrimidin-2-yl)thiazol-2-amine (8.5 pmol, 2.9 mg, 8%) as a yellow solid.

UPLC-MS: RT=0.88 min; MS m/z ES⁺=343.

### Example 6

### 4-(3-(5-Fluoro-2-methoxyphenyl)-1H-pyrazol-4-yl)-N-(pyrimidin-2-yl)thiazol-2-amine (Final Compound 1-31)

### Ethyl 3-bromo-M-pyrazole-4-carboxylate

According to Scheme 6, Step 1: To a solution of tert-BuONO (156 mmol, 16.5 g) in acetonitrile (300 mL) was added CuBr₂ (156 mmol, 34.8 g). After the mixture was stirred at rt for 1 h under nitrogen, ethyl 3-amino-1H-pyrazole-4-carboxylate (129 mmol, 20.0 g) was added portionwise over 30 min. The reaction mixture was stirred at it for 30 min and then was allowed to warm up to 70° C. and was stirred for another 2 h. After cooling to rt, the solvent was removed under reduced pressure. The residue was diluted with EtOAc (1 L) and was washed with brine (200 mL×3). The organic layer was dried over MgSO₄, filtered, and concentrated to afford ethyl 3-bromo-1H-pyrazole-4-carboxylate (106 mmol, 23.2 g, 81%) and was used without any purification.

LC-MS: m/z ES⁺=219, 221.

### Ethyl 3-bromo-1-(4-methoxybenzyl)-1H-pyrazole-4-carboxylate

According to Scheme 6, Step 2: A mixture of ethyl 3-bromo-1H-pyrazole-4-carboxylate (106 mmol, 23.2 g), PMBCl (116 mmol, 18.2 g) and K₂CO₃ (159 mmol, 21.9 g) in acetonitrile (250 mL) was refluxed for 18 h. After cooling to rt, the reaction mixture was filtered and washed with DCM (200 mL×3). The filtrate was collected and was concentrated under reduced pressure. The residue was purified by flash chromatography over silica gel PE/EtOAc (30:1 to 10:1) to give ethyl 3-bromo-1-(4-methoxybenzyl)-1H-pyrazole-4-carboxylate as an off-white solid (13.2 g, 37%).

LC-MS: m/z ES⁺=339, 341.

### 3-Bromo-1-(4-methoxybenzyl)-1H-pyrazole-4-carboxylic acid

According to Scheme 6, Step 3: To a solution of ethyl 3-bromo-1-(4-methoxybenzyl)-1H-pyrazole-4-carboxylate (38 mmol, 13.0 g) in MeOH (60 mL) was added 4 M NaOH solution (60 mL). The reaction mixture was refluxed for 2 h. After cooling to rt, conc. HCl was added to the reaction mixture to reach pH=2-3 and the aqueous phase was extracted with EtOAc (150 mL×3). The combined organic layer was washed with water, dried over MgSO₄, filtered, and concentrated in vacuum to produce 3-bromo-1-(4-methoxybenzyl)-1H-pyrazole-4-carboxylic acid (35.3 mmol, 11.0 g, 93%) and was used without any purification.

LC-MS: m/z ES⁺=311, 313.

### 2-Bromo-1-(3-bromo-1-(4-methoxybenzyl)-1H-pyrazol-4-yl)ethanone

According to Scheme 6, Step 4: To a solution of 3-bromo-1-(4-methoxybenzyl)-1H-pyrazole-4-carboxylic acid (35.3 mmol, 11.0 g) and five drops of DMF in DCM (100 mL) was added oxalyl chloride (70 mmol, 8.90 g) dropwise at 0° C. The reaction mixture was allowed to warm up to rt and was stirred for 3 h. After concentration, the residue was treated with toluene and was co-evaporated to dryness to yield 3-bromo-1-(4-methoxybenzyl)-1H-pyrazole-4-carbonyl chloride.

To a solution of 3-bromo-1-(4-methoxybenzyl)-1H-pyrazole-4-carbonyl chloride (35 mmol) in acetonitrile (100 mL) was added a solution of TMSCHN₂ (70 mmol, 35 mL) dropwise at 0° C. and then the reaction mixture was allowed to warm up to rt and was stirred overnight. After cooling the reaction mixture to 0° C., HBr/AcOH (17 mL) was added dropwise and the reaction mixture was allowed to warm up to rt and was stirred for 6 h. The mixture was concentrated in vacuum to afford 2-bromo-1-(3-bromo-1-(4-methoxybenzyl)-1H-pyrazol-4-yl)ethanone (32.8 mmol, 12.7 g, 93%) which was used without any purification.

LC-MS: m/z ES⁺=387, 389, 391.

### 4-(3-Bromo-1-(4-methoxybenzyl)-1H-pyrazol-4-yl)-N-(pyrimidin-2-yl)thiazol-2-amine

According to Scheme 6, Step 5: A mixture of 2-bromo-1-(3-bromo-1-(4-methoxybenzyl)-1H-pyrazol-4-yl)ethanone (32.8 mmol, 12.7 g) and 1-(pyrimidin-2-yl)-thiourea (33 mmol, 5.1 g) in EtOH (100 mL) was refluxed for 1 h. After cooling to rt, the reaction mixture was filtered and was washed with MeOH (20 mL×3). The filtrate was collected and concentrated under reduced pressure. The residue was purified by flash chromatography over silica gel DCM/MeOH (30:1) to give 4-(3-bromo-1-(4-methoxybenzyl)-1H-pyrazol-4-yl)-N-(pyrimidin-2-yl)thiazol-2-amine (13.8 mmol, 6.20 g, 42%) as a yellow solid.

LC-MS: m/z ES⁺=443, 445.

### 4-(3-(5-Fluoro-2-methoxyphenyl)-1-(4-methoxybenzyl)-1H-pyrazol-4-yl)-N-(pyrimidin-2-yl)thiazol-2-amine

According to Scheme 6, Step 6: To a suspension of 4-(3-bromo-1-(4-methoxybenzyl)-1H-pyrazol-4-yl)-N-(pyrimidin-2-yl)thiazol-2-amine (0.22 mmol, 100 mg) and 5-fluoro-2-methoxy phenylboronic acid (0.33 mmol, 56 mg) in dioxane (4 mL)/H₂O (1 mL) were added Pd(PPh₃)₄ (33 μmol 38 mg) and NaHCO₃ (1.20 mmol, 101 mg). The resulting mixture was stirred at 120° C. under N₂ atmosphere overnight. After cooling to rt, the reaction mixture was filtered, the solid was washed with MeOH (20 mL) and the combined filtrate was concentrated under reduced pressure. Then the residue was purified by preparative TLC using DCM/MeOH (50:1) as eluent to give 4-(3-(5-fluoro-2-methoxyphenyl)-1-(4-methoxybenzyl)-1H-pyrazol-4-yl)-N-(pyrimidin-2-yl)thiazol-2-amine (0.13 mmol, 63 mg, 59%).

LC-MS: m/z ES⁺=489.

### 4-(3-(5-Fluoro-2-methoxyphenyl)-1H-pyrazol-4-yl)-N-(pyrimidin-2-yl)thiazol-2-amine

According to Scheme 6, Step 7: 4-(3-(5-Fluoro-2-methoxyphenyl)-1-(4-methoxybenzyl)-1H-pyrazol-4-yl)-N-(pyrimidin-2-yl)thiazol-2-amine (0.13 mmol, 63 mg) was dissolved in TFA (2 mL) and the solution was stirred at 100° C. under microwave conditions for 20 min. After cooling to rt, the mixture was concentrated and the residue was purified by preparative HPLC to give 4-(3-(5-fluoro-2-methoxyphenyl)-1H-pyrazol-4-yl)-N-(pyrimidin-2-yl)thiazol-2-amine (13 μmol, 8 mg, 10%).

LC-MS: RT=2.72 min; m/z ES⁺=369.

¹H-NMR: (CD₃OD, 400 MHz) δ 8.62 (d, 2H, J=4.8 Hz), 8.00 (s, 1H), 7.07-7.22 (m, 13H), 7.02 (t, 1H, J=4.8 Hz), 6.47 (s, 1H), 3.67 (s, 3H).

### Example 7

### 3-(3-(2-(Pyrimidin-2-ylamino)thiazol-4-yl)-1H-pyrazol-4-yl) benzonitrile (Final Compound 1-43)

### 4-(4-Bromo-1-(4-methoxybenzyl)-1H-pyrazol-3-yl)thiazol-2-amine

According to Scheme 7, Step 1: A solution of 2-bromo-1-(3-bromo-1-(4-methoxybenzyl)-1H-pyrazol-4-yl)ethanone (5.50 mmol, 2.12 g) and thiourea (5.50 mmol, 0.42 g) in EtOH (20 mL) was refluxed for 1 h. After cooling to rt, the reaction mixture was concentrated under reduced pressure. The residue was dissolved in DCM (100 mL) and the solution pH was adjusted to 8-9 with a saturated solution of NaHCO₃. The organic layer was separated, washed with brine, dried over MgSO₄, filtered, and concentrated to give 4-(4-bromo-1-(4-methoxybenzyl)-1H-pyrazol-3-yl)thiazol-2-amine (4.12 mmol, 1.50 g, 75%) which was used without any purification.

LC-MS: m/z ES⁺=365, 367.

### 4-(3-Bromo-1-(4-methoxybenzyl)-1H-pyrazol-4-yl)thiazol-2-amino-di-(tert-butoxycarbonyl)

According to Scheme 7, Step 2: A solution of 4-(4-bromo-1-(4-methoxybenzyl)-1H-pyrazol-3-yl)thiazol-2-amine (4.10 mmol, 1.50 g), Boc₂O (12.3 mmol, 2.70 g) and Et₃N (12.3 mmol, 1.20 g) in DCM (20 mL) was stirred at rt overnight. After evaporation of the solvent, the residue was purified by flash chromatography over silica gel PE/EtOAc (50:10) to give 4-(3-bromo-1-(4-methoxybenzyl)-1H-pyrazol-4-yl)thiazol-2-amino-di-(tert-butoxycarbonyl) (1.30 mmol, 720 mg, 31%).

LC-MS: m/z ES⁺=565, 567.

### 3-(4-(2-Amino-di-(tert-butoxycarbonyl)-thiazol-4-yl)-1-(4-methoxybenzyl)-1H-pyrazol-3-yl)benzonitrile

According to Scheme 7, Step 3: To a suspension of 4-(3-bromo-1-(4-methoxybenzyl)-1H-pyrazol-4-yl)thiazol-2-amino-di-(tert-butoxycarbonyl) (1.30 mmol, 720 mg) and 3-cyano-phenylboronic acid (1.90 mmol, 280 mg) in dioxane (10 mL)/H₂O (2 mL) were added Pd(PPh₃)₄ (0.19 mmol, 230 mg) and NaHCO₃ (5.20 mmol, 436 mg). Then the reaction mixture was stirred at 120° C. under N₂ atmosphere overnight. After cooling to rt, the reaction mixture was filtered and the solid was washed with MeOH (20 mL) and the combined filtrate was concentrated under reduced pressure. The residue was purified by flash chromatography over silica gel PE/EtOAc (40:10) to give 34442-amino-di-(tert-butoxycarbonyl)-thiazol-4-yl)-1-(4-methoxybenzyl)-1H-pyrazol-3-yl)benzonitrile (0.88 mmol, 520 mg, 68%).

LC-MS: m/z ES⁺=588.

### 3-(3-(2-Aminothiazol-4-yl)-1-(4-methoxybenzyl)-1H-pyrazol-4-yl)benzonitrile

According to Scheme 7, Step 4: TFA (3.5 mL) was added to a solution of 34442-amino-di-(tert-butoxycarbonyl)-thiazol-4-yl)-1-(4-methoxybenzyl)-1H-pyrazol-3-yl) benzonitrile (0.88 mmol, 520 mg) in DCM (20 mL) and the reaction mixture was stirred at rt for 4 h. After evaporation of the solvent, the residue was purified by preparative HPLC to give 3-(3-(2-aminothiazol-4-yl)-1-(4-methoxybenzyl)-1H-pyrazol-4-yl)benzonitrile (0.31 mmol, 120 mg, 35%).

LC-MS: m/z ES⁺=388.

### 3-(1-(4-Methoxybenzyl)-3-(2-(pyrimidin-2-ylamino)thiazol-4-yl)-1H-pyrazol-4-yl) benzonitrile

According to Scheme 7, Step 5: To a suspension of 3-(3-(2-aminothiazol-4-yl)-1-(4-methoxybenzyl)-1H-pyrazol-4-yl)benzonitrile (0.26 mmol, 100 mg) in dioxane (5 mL), were added 2-chloropyrimidine (0.52 mmol, 60 mg), Pd₂dba₃ (0.04 mmol, 37 mg), Xantphos (0.08 mmol, 46 mg) and Cs₂CO₃ (0.52 mmol, 169 mg). The reaction mixture was refluxed under N₂ for 2 h. After cooling to rt, the mixture was diluted with MeOH (20 mL), filtered and concentrated under reduced pressure. The residue was purified by preparative TLC with DCM/MeOH (50:1) as eluent to give 3-(1-(4-methoxybenzyl)-3-(2-(pyrimidin-2-ylamino)thiazol-4-yl)-1H-pyrazol-4-yl) benzonitrile (0.11 mmol, 50 mg, 41%).

LC-MS: m/z ES⁺=466.

### 3-(3-(2-(Pyrimidin-2-ylamino)thiazol-4-yl)-1H-pyrazol-4-yl)benzonitrile

According to Scheme 7, Step 6: 3-(1-(4-Methoxybenzyl)-3-(2-(pyrimidin-2-ylamino)thiazol-4-yl)-1H-pyrazol-4-yl)benzonitrile (0.10 mmol, 50 mg) was dissolved in TFA (2 mL) and the mixture was stirred at 80° C. under microwave conditions for 20 min. After cooling to rt, the mixture was concentrated and the residue was purified by preparative HPLC to give 3-(3-(2-(pyrimidin-2-ylamino)thiazol-4-yl)-1H-pyrazol-4-yl)benzonitrile (23 μmol, 8 mg, 23%).

LC-MS: RT=3.05 min; m/z ES⁺=346;

¹H-NMR: (DMSO-d₆, 400 MHz): δ 11.77 (s, 1H), 8.63 (d, 2H, J=4.8 Hz), 7.97-8.00 (m, 3H), 7.80 (d, 1H, J=7.2 Hz), 7.60 (t, 1H, J=8.0 Hz), 7.03 (t, 1H, J=4.8 Hz), 6.87 (s, 1H).

### Example 8

### 1-(3-(4-(2-(4-Methylpyrimidin-2-ylamino)thiazol-4-yl)-1H-pyrazol-3-yl)phenyl)pyrrolidin-3-ol (Final Compound 1-42)

### 1-(3-Bromophenyl)pyrrolidin-3-ol

According to Scheme 8, Step 1: A mixture of 1,3-dibromobenzene (42.5 mmol, 10.0 g), pyrrolidin-3-ol (38.6 mmol, 3.20 g), t-BuOK (70.9 mmol, 7.95 g), Pd₂ dba₃ (1.93 mmol, 1.70 g) and BINAP (100 mg) in toluene (80 mL) was heated for 3 h at 80° C. After evaporation of the solvent, the residue was purified by flash chromatography over silica gel PE/EtOAc (1:1) to give 1-(3-bromophenyl)pyrrolidin-3-ol (7.95 mmol, 2.10 g, 21%).

LC-MS: m/z ES⁺=242, 244.

### 1-(3-Bromophenyl)-3-(tert-butyldimethylsilyloxy)pyrrolidine

According to Scheme 8, Step 2: A mixture of 1-(3-bromophenyl)pyrrolidin-3-ol (4.1 mmol, 1.0 g), TBSCl (5.00 mmol, 746 mg) and imidazole (16.5 mmol, 1.12 g) in acetonitrile (12 mL) was stirred for 3 h at rt. After evaporation, the residue was purified by flash chromatography over silica gel PE/EtOAc (10:1) to give 1-(3-bromophenyl)-3-(tert-butyldimethylsilyloxy)pyrrolidine (3.03 mmol, 1.10 g, 74%).

LC-MS: m/z ES⁺=356, 358.

### 3-(tert-Butyldimethylsdyloxy)-1-(3-(4,4,5,5-tetramethyl-1,3,2-dioxaborolan-2-yl) phenyl)pyrrolidine

According to Scheme 8, Step 3: A mixture of 1-(3-bromophenyl)-3-(tert-butyldimethylsilyloxy)pyrrolidine (3.03 mmol, 1.10 g), bis(pinacolato)diboron (3.60 mmol, 910 mg), KOAc (9.00 mmol, 900 mg) and PdCl₂(dppf) (0.15 mmol, 710 mg) in DMF (12 mL) was heated for 3.5 h at 100° C. Then the mixture was filtered, diluted with water, extracted with EtOAc, washed with water, dried over Na₂SO₄, filtered and concentrated under reduced pressure. The residue was purified by flash chromatography over silica gel PE/EtOAc (10:1) to give 3-(tert-butyldimethylsilyloxy)-1-(3-(4,4,5,5-tetramethyl-1,3,2-dioxaborolan-2-yl)phenyl)pyrrolidine (2.27 mmol, 0.90 g, 75%).

LC-MS: m/z ES⁺=404.

### 4-(3-(3-(3-(tert-Butyldimethylsilyloxy)pyrrolidin-1-yl)phenyl)-1-(4-methoxybenzyl)-1H-pyrazol-4-yl)-N-(4-methylpyrimidin-2-yl)thiazol-2-amine

According to Scheme 8, Step 4: A mixture of 4-(3-bromo-1-(4-methoxybenzyl)-1H-pyrazol-4-yl)-N-(4-methylpyrimidin-2-yl)thiazol-2-amine (0.55 mmol, 230 mg), 3-(tert-butyldimethylsilyloxy)-1-(3-(4,4,5,5-tetramethyl-1,3,2-dioxaborolan-2-yl)phenyl)pyrrolidine (0.65 mmol, 290 mg), NaHCO₃ (2.20 mmol, 184 mg), and Pd(PPh₃)₄ (27 μmol, 30 mg) in dioxane (10 mL) and H₂O (5 mL) was heated for 9 h at 120° C. After cooling to rt, the mixture was filtered and concentrated under reduced pressure. The residue was purified by preparative TLC using PE/EtOAc (1:1) as eluent to give 4-(3-(3-(3-(tert-butyldimethylsilyloxy)pyrrolidin-1-yl)phenyl)-1-(4-methoxybenzyl)-1H-pyrazol-4-yl)-N-(4-methylpyrimidin-2-yl)thiazol-2-amine (0.12 mmol, 80 mg, 22%).

LC-MS: m/z ES⁺=655.

### 1-(3-(4-(2-(4-Methylpyrimidin-2-ylamino)thiazol-4-yl)-1H-pyrazol-3-yl)phenyl) pyrrolidin-3-ol

According to Scheme 8, Step 5: To a solution of 4-(3-(3-(3-(tert-butyldimethylsilyloxy)pyrrolidin-1-yl)phenyl)-1-(4-methoxybenzyl)-1H-pyrazol-4-yl)-N-(4-methylpyrimidin-2-yl)thiazol-2-amine (0.12 mmol, 80 mg) in TFA (2 mL) was stirred at 120° C. for 10 min under microwave conditions. After cooling to rt, the mixture was purified via preparative HPLC to give 1-(3-(4-(2-(4-methylpyrimidin-2-ylamino)thiazol-4-yl)-1H-pyrazol-3-yl)phenyl)pyrrolidin-3-ol (24 pmol, 10 mg, 20%).

LC-MS: RT=2.63 min; m/z ES⁺=420.

¹H-NMR (CD₃OD, 400 MHz): δ 8.42 (d, 1H, J=5.2 Hz), 7.91 (s, 1H), 7.21-7.24 (t, 1H, J=7.2 Hz), 6.93 (d, 1H, J=5.2 Hz), 6.79 (d, 2H, J=7.2 Hz), 6.71 (s, 1H) 6.62 (s, 1H), 4.47 (s, 1H), 3.29 (s, 2H), 3.15-3.17 (t, 2H, J=10.0 Hz), 2.53 (s, 3H), 2.08-2.13 (t, 1H, J=4.8 Hz), 1.99 (d, 1H, J=3.6 Hz).

### Example 9

### Methyl 3-(4-(2-(4-methylpyrimidin-2-ylamino)thiazol-4-yl)-1H-pyrazol-3-yl)-2,5-dihydro-1H-pyrrole-1-carboxylate (Final Compound 1-39) tert-Butyl 3-(1-(4-methoxybenzyl)-4-(2-(4-methylpyrimidin-2-ylamino)thiazol-4-yl)-1H-pyrazol-3-yl)-2,5-dihydro-1H-pyrrole-1-carboxylate

According to Scheme 9, Step 1: To a solution of 4-(3-bromo-1-(4-methoxybenzyl)-1H-pyrazol-4-yl)-N-(4-methylpyrimidin-2-yl)thiazol-2-amine (1.09 mmol, 0.50 g) in dioxane/H₂O (50 mL/10 mL) were added tert-butyl 3-(4,4,5,5-tetramethyl-1,3,2-dioxaborolan-2-yl)-2,5-dihydro-1H-pyrrole-1-carboxylate (1.64 mmol, 0.48 g), Pd(PPh₃)₄ (0.11 mmol, 0.13 g) and NaHCO₃ (4.38 mmol, 0.37 g). The resulting mixture was stirred at reflux under nitrogen atmosphere overnight. After cooling to rt, the mixture was filtered and concentrated under reduced pressure. The residue was purified by flash chromatography over silica gel PE/EtOAc (1:5) to give tert-butyl 3-(1-(4-methoxybenzyl)-4-(2-(4-methylpyrimidin-2-ylamino)thiazol-4-yl)-1H-pyrazol-3-yl)-2,5-dihydro-1H-pyrrole-1-carboxylate (0.31 mmol, 0.17 g, 29%) as a yellow solid.

LC-MS: m/z ES⁺=546.

### 4-(3-(2,5-Dihydro-1H-pyrrol-3-yl)-1-(4-methoxybenzyl)-1H-pyrazol-4-yl)-N-(4-methyl pyrimidin-2-yl)thiazol-2-amine

According to Scheme 9, Step 2: A solution of tert-butyl 3-(1-(4-methoxybenzyl)-4-(2-(4-methylpyrimidin-2-ylamino)thiazol-4-yl)-1H-pyrazol-3-yl)-2,5-dihydro-1H-pyrrole-1-carboxylate (0.18 mmol, 0.10 g) in HCl/MeOH (4 M, 2 mL) was stirred at rt for 2 h. Then the solvent was removed under reduced pressure to give 4-(3-(2,5-dihydro-1H-pyrrol-3-yl)-1-(4-methoxybenzyl)-1H-pyrazol-4-yl)-N-(4-methylpyrimidin-2-yl)thiazol-2-amine (0.17 mmol, 75 mg, 92%) which was used for the next step without any purification.

LC-MS: m/z ES⁺=446.

### Methyl 3-(1-(4-methoxybenzyl)-4-(2-(4-methylpyrimidin-2-ylamino)thiazol-4-yl)-1H-pyrazol-3-yl)-2,5-dihydro-1H-pyrrole-1-carboxylate

According to Scheme 9, Step 3: To a solution of 4-(3-(2,5-dihydro-1H-pyrrol-3-yl)-1-(4-methoxybenzyl)-1H-pyrazol-4-yl)-N-(4-methylpyrimidin-2-yl)thiazol-2-amine (0.11 mmol, 50 mg) in DCM (2 mL) were added methyl chloroformate (0.12 mmol, 11.6 mg) and Et₃N (0.34 mmol, 34 mg), the resulting mixture was stirred at rt overnight. Then the solvent was removed under reduced pressure to give methyl 3-(1-(4-methoxybenzyl)-4-(2-(4-methylpyrimidin-2-ylamino)thiazol-4-yl)-1H-pyrazol-3-yl)-2,5-dihydro-1H-pyrrole-1-carboxylate (83 μmol, 42 mg, 75%) which was used for the next step without any purification.

LC-MS: m/z ES⁺=504.

### Methyl 3-(4-(2-(4-methylpyrimidin-2-ylamino)thiazol-4-yl)-1H-pyrazol-3-yl)-2,5-dihydro-1H-pyrrole-1-carboxylate

According to Scheme 9, Step 4: A solution of methyl 3-(1-(4-methoxybenzyl)-4-(2-(4-methylpyrimidin-2-ylamino)thiazol-4-yl)-1H-pyrazol-3-yl)-2,5-dihydro-1H-pyrrole-1-carboxylate (83 μmol, 42 mg) in TFA (2 mL) was stirred at 120° C. for 10 min under under microwave conditions. After cooling to rt, the mixture was concentrated under reduced pressure and the residue was purified by preparative HPLC to give methyl 3-(4-(2-(4-methylpyrimidin-2-ylamino)thiazol-4-yl)-1H-pyrazol-3-yl)-2,5-dihydro-1H-pyrrole-1-carboxylate (56 umol, 22 mg, 68%).

LC-MS: RT=2.43 min; m/z ES⁺=384.

¹H-NMR (CD₃OD, 400 MHz): 8.46 (d, 1H, J=5.6 Hz), 7.83 (d, 1H, J=3.6 Hz), 7.01 (s, 1H), 6.94 (d, 1H, J=5.2 Hz), 4.51 (br, 2H), 4.27 (br, 2H), 3.71 (d, 3H, J=3.2 Hz), 2.56 (d, 3H).

### Example 10

### 4-(3-Morpholino-1H-pyrazol-4-yl)-N-(pyridin-2-yl)thiazol-2-amine (Final Compound 1-13)

### 3-Chloro-N-methoxy-1-(4-methoxybenzyl)-N-methyl-1H-pyrazole-4-carboxamide

According to Scheme 10, Step 1: BuLi 2.5 M (72.6 mmol, 29 mL) was added to a solution of diisopropylamine (72.6 mmol, 7.35 g) in THF (50 mL) at −78° C. and the reaction mixture was stirred at −78° C. for 5 min and then at rt. The resulting LDA solution was added at −78° C. to a solution of N-methoxy-1-(4-methoxybenzyl)-N-methyl-1H-pyrazole-4-carboxamide (36.3 mmol, 10.0 g) in THF (5 mL) and the reaction mixture was stirred for 5 min at −78° C. Then a solution of hexachloroethane (72.6 mmol, 17.2 g) in THF (5 mL) was added to the reaction mixture at −78° C. and the solution was stirred for 5 min at −78° C. and for 1 h at rt. The reaction mixture was quenched with water (50 mL) and the aqueous phase was extracted with EtOAc. The organic phase was dried over MgSO₄, was filtered and was concentrated to yield a brown oil. The resulting crude product was purified by flash chromatography over silica gel using cyclohexane/EtOAc (100:0 to 50:50) as eluent to yield after evaporation 3-chloro-N-methoxy-1-(4-methoxybenzyl)-N-methyl-1H-pyrazole-4-carboxamide (20.3 mmol, 6.30 g, 56%) as a beige solid.

UPLC-MS: RT=0.82 min; MS m/z ES⁺=310.

### N-Methoxy-1-(4-methoxybenzyl)-N-methyl-3-morpholino-1H-pyrazole-4-carboxamide

According to Scheme 10, Step 2: Morpholine (22.6 mmol, 1.97 g) was added to a solution of 3-chloro-N-methoxy-1-(4-methoxybenzyl)-N-methyl-1H-pyrazole-4-carboxamide (2.26 mmol, 700 mg) in NMP (20 mL) and the reaction mixture was stirred at 180° C. for 2 h under microwave heating. EtOAc was then added and the organic phase was washed with water. The organic layer was dried over MgSO₄, was filtered and was concentrated. The crude compound was purified by flash chromatography with silica gel using cyclohexane/EtOAc (100:0 to 0:100) as eluent to yield N-methoxy-1-(4-methoxybenzyl)-N-methyl-3-morpholino-1H-pyrazole-4-carboxamide (1.11 mmol, 400 mg, 49%).

UPLC-MS: RT=0.81 min; MS m/z ES⁺=361.

4-(3-Morpholino-1H-pyrazol-4-yl)-N-(pyridin-2-yl)thiazol-2-amine was obtained as a yellow solid following the same experimental part as described for Example 4. UPLC-MS: RT=0.61 min; MS m/z ES⁺=329.

### Example 11

### (4-(2-(4-Methylpyrimidin-2-ylamino)thiazol-4-yl)-1H-pyrazol-3-yl)(phenyl)methanone (Final Compound 1-25)

### Ethyl 3-(hydroxy(phenyl)methyl)-1-(4-methoxybenzyl)-1H-pyrazole-4-carboxylate

According to Scheme 11, Step 1: At −78° C., under nitrogen, butyllithium 2.5 M (15.0 mmol, 5.99 mL) was added to a solution of diisopropylamine (15.0 mmol, 2.10 mL) in THF (8 mL). The reaction mixture was stirred for 30 min at 0° C. The resulting LDA solution was added at −78° C. to a solution of ethyl 1-(4-methoxybenzyl)-1H-pyrazole-4-carboxylate (11.5 mmol, 3.00 g) in THF (50 mL). After 15 min, benzaldehyde (23.0 mmol, 2.34 mL) was added and then the reaction mixture was stirred at rt for 1 h. The reaction mixture was diluted with EtOAc and was washed with a saturated aqueous solution of NH₄Cl. The combined organic phases were dried over MgSO₄, filtered and evaporated to yield a brown oil. The crude compound was purified by flash chromatography with silica gel using cyclohexane/EtOAc (90:10 to 70:30) as eluent to yield ethyl 3-(hydroxy(phenyl)methyl)-1-(4-methoxybenzyl)-1H-pyrazole-4-carboxylate (6.41 mmol, 2.35 g, 56%) as a yellow oil.

UPLC-MS: RT=1.09 min; MS m/z ES⁺=367.

### Ethyl 3-benzoyl-1-(4-methoxybenzyl)-1H-pyrazole-4-carboxylate

According to Scheme 11, Step 2: A mixture of ethyl 3-(hydroxy(phenyl)methyl)-1-(4-methoxybenzyl)-1H-pyrazole-4-carboxylate (6.41 mmol, 2.35 g) and Dess Martin reagent (7.70 mmol, 3.26 g) in DCM (64 mL) was stirred at rt overnight. The precipitate was filtered off and then the filtrate was diluted with DCM and was washed with water. The combined organic phases were dried over MgSO₄, filtered and solvents were evaporated. Ethyl 3-benzoyl-1-(4-methoxybenzyl)-1H-pyrazole-4-carboxylate (6.41 mmol, 2.34 g) was obtained and was used without any purification.

UPLC-MS: RT=1.11 min; MS m/z ES⁺=365.

(4-(2-(4-Methylpyrimidin-2-ylamino)thiazol-4-yl)-1H-pyrazol-3-yl)(phenyl)methanone was obtained as a brown solid following the same experimental part as described for Example 6, Step 3 to 5 and 7.

M.p.: 254-255° C.;

UPLC-MS: RT=0.90 min; MS m/z ES⁺=363;

¹H-NMR (DMSO-d₆, 300 MHz): 8.47 (1H, d, 5 Hz), 8.18 (1H, s), 7.95-7.98 (2H, m), 7.61-7.64 (1H, m), 7.52 (3H, t, 7 Hz), 6.91 (1H, d, 5 Hz), 2.44 (3H, s).

### Example 12

### (4-(2-(4-Methylpyrimidin-2-ylamino)thiazol-4-yl)-1H-pyrazol-3-yl)(phenyl)methanol (Final Compounds 1-26)

According to Scheme 12: To a solution of crude (4-(2-(4-methylpyrimidin-2-ylamino)thiazol-4-yl)-1H-pyrazol-3-yl)(phenyl)methanone (0.77 mmol, 280 mg) in EtOH (4 mL) was added portionwise, at 0° C., NaBH₄ (1.55 mmol, 58.5 mg). The reaction mixture was stirred at 0° C. for 1 h and then 1 h at rt. The reaction mixture was diluted with water and the aqueous phase was extracted with EtOAc. The combined organic phases were dried over MgSO₄, filtered and solvents were evaporated. The crude compound was purified by preparative HPLC to yield (4-(2-(4-methylpyrimidin-2-ylamino)thiazol-4-yl)-1H-pyrazol-3-yl)(phenyl)methanol (95 μmol, 34.5 mg, 12%) as a white solid.

M.p.: 249.5° C.;

UPLC-MS: RT=0.82 min; MS m/z ES⁺=365;

¹H-NMR (CD₃OD, 300 MHz): 11.85 (1H, s), 8.49 (1H, d, 5 Hz), 7.88 (1H, s), 7.49-7.51 (2H, m), 7.25 (2H, t, 8 Hz), 7.15 (2H, t, 8 Hz), 7.05 (1H, s), 6.92 (1H, d, 5 Hz), 6.54 (1H, s), 3.36 (1H, s), 2.45 (3H, s).

### Example 13

### N-Methyl-4-(2-(4-methylpyrimidin-2-ylamino)thiazol-4-yl)-1H-pyrazole-5-carboxamide (Final Compound 1-32)

### Ethyl 4-iodo-1-(4-methoxybenzyl)-1H-pyrazole-3-carboxylate and ethyl 4-iodo-1-(4-methoxybenzyl)-1H-pyrazole-5-carboxylate

According to Scheme 13, Step 1: A mixture of ethyl 4-iodo-1H-pyrazole-5-carboxylate (164 mmol, 43.6 g), PMBCl (177 mmol, 24.0 mL) and K₂CO₃ (246 mmol, 34.0 g) in acetonitrile (328 mL) was stirred at 60° C. overnight. After cooling to rt, the reaction mixture was filtered and the filtrate was concentrated under reduced pressure to give ethyl 4-iodo-1-(4-methoxybenzyl)-1H-pyrazole-3-carboxylate and ethyl 4-iodo-1-(4-methoxybenzyl)-1H-pyrazole-5-carboxylate (160 mmol, 61.9 g, 98%).

UPLC-MS: RT=1.05 and 1.19 min; MS m/z ES⁺=387.

### 4-Iodo-1-(4-methoxybenzyl)-1H-pyrazole-3-carboxylic acid and 4-iodo-1-(4-methoxybenzyl)-1H-pyrazole-5-carboxylic acid

According to Scheme 13, Step 2: 3 M NaOH solution (134 mL) was slowly added to a solution of ethyl 4-iodo-1-(4-methoxybenzyl)-1H-pyrazole-3-carboxylate and ethyl 4-iodo-1-(4-methoxybenzyl)-1H-pyrazole-5-carboxylate (160 mmol, 61.9 g) in MeOH (400 mL) and the reaction mixture was stirred at 50° C. for 4 h. After evaporation of the solvent, the reaction mixture was partitioned between 1 M NaOH and Et₂O. The organic layer was washed with 1 M NaOH solution. The aqueous layer was acidified with conc. HCl to reach pH=2-3 and the aqueous phase was extracted with DCM. The combined organic layers was washed with water, dried over MgSO₄, filtered, and concentrated in vacuum to yield 4-iodo-1-(4-methoxybenzyl)-1H-pyrazole-3-carboxylic acid and 4-iodo-1-(4-methoxybenzyl)-1H-pyrazole-5-carboxylic acid (137 mmol, 48.7 g, 85%) as an off-white solid and was used without any purification.

UPLC-MS: RT=0.79 and 0.87 min; MS m/z ES⁺=359.

### 4-Iodo-N-methoxy-1-(4-methoxybenzyl)-N-methyl-1H-pyrazole-3-carboxamide and 4-iodo-N-methoxy-1-(4-methoxybenzyl)-N-methyl-1H-pyrazole-5-carboxamide

According to Scheme 13, Step 3: Oxalyl dichloride (275 mmol, 23.6 mL) followed by few drops of DMF were added to a solution of 4-iodo-1-(4-methoxybenzyl)-1H-pyrazole-3-carboxylic acid and 4-iodo-1-(4-methoxybenzyl)-1H-pyrazole-5-carboxylic acid (137 mmol, 49.2 g) in DCM (250 mL). When no more gas was generated, the solution was evaporated to dryness and then the residue was diluted in DCM (50 mL). The resulting acid chloride solution was added to a solution of N,O-dimethylhydroxylamine hydrochloride (165 mmol, 16.1 g) and Et₃N (343 mmol, 48.2 mL) in dry DCM (250 mL), at 0° C. The reaction mixture was stirred at rt for 2 h. Then the reaction mixture was diluted with DCM and was washed with water, 1M HCl cold solution, 1M NaOH solution and brine. The combined organic phases were dried over MgSO₄, filtered and evaporated to dryness to yield 4-iodo-N-methoxy-1-(4-methoxybenzyl)-N-methyl-1H-pyrazole-3-carboxamide and 4-iodo-N-methoxy-1-(4-methoxybenzyl)-N-methyl-1H-pyrazole-5-carboxamide (133 mmol, 53.7 g, 97%) as a brown oil which solidified. The compound was used in the next step without any purification.

UPLC-MS: RT=0.89 and 0.91 min; MS m/z ES⁺=402.

### 4-Acetyl-N-methoxy-1-(4-methoxybenzyl)-N-methyl-1H-pyrazole-3-carboxamide

According to Scheme 13, Step 4: A mixture of 4-iodo-N-methoxy-1-(4-methoxybenzyl)-N-methyl-1H-pyrazole-3-carboxamide and 4-iodo-N-methoxy-1-(4-methoxybenzyl)-N-methyl-1H-pyrazole-5-carboxamide (64.9 mmol, 26.0 g), 1-(vinyloxy)butane (154 mmol, 20 mL), Et₃N (195 mmol, 27.1 mL) and PdCl₂(dppf) (0.65 mmol, 0.53 g) in DMF (110 mL) was stirred at 80° C. for 2 h. As the reaction was not complete, PdCl₂(dppf) (0.32 mmol, 0.53 g) and 1-(vinyloxy)butane (77.0 mmol, 10 mL) were added and the reaction mixture was stirred at 80° C. overnight. Then HCl 3 M was added at 0° C. and the reaction mixture was stirred at rt. The reaction mixture was filtered through a pad of celite and washed with EtOAc. The crude residue was diluted with EtOAc and was washed with water. The combined organic phases were dried over MgSO₄, filtered and the solvents were evaporated. The crude compound was purified by flash chromatography with silica gel using cyclohexane/EtOAc (50:50 to 10:90) as eluent to yield 4-acetyl-N-methoxy-1-(4-methoxybenzyl)-N-methyl-1H-pyrazole-3-carboxamide (9.45 mmol, 3.00 g, 15%).

UPLC-MS: RT=0.74 min; MS m/z ES⁺=318.

### 4-(2-Bromoacetyl)-N-methoxy-1-(4-methoxybenzyl)-N-methyl-1H-pyrazole-3-carboxamide

According to Scheme 13, Step 5: To a solution of 4-acetyl-N-methoxy-1-(4-methoxybenzyl)-N-methyl-1H-pyrazole-3-carboxamide (9.45 mmol, 3.00 g) in CHCl₃ (95 mL) was added trimethylphenylammomium tribromide (7.56 mmol, 2.93 g) and the reaction mixture was stirred for 1 h at 40° C. The reaction mixture was diluted with water and the aqueous phase was extracted with DCM. The combined organic phases were dried over MgSO₄, filtered and solvents were evaporated to yield 4-(2-bromoacetyl)-N-methoxy-1-(4-methoxybenzyl)-N-methyl-1H-pyrazole-3-carboxamide (7.56 mmol, 3.75 g, 100%) which was used in the next step without any purification.

UPLC-MS: RT=0.86 min; MS m/z ES⁺=396, 398.

### N-Methoxy-1-(4-methoxybenzyl)-N-methyl-4-(2-(4-methylpyrimidin-2-ylamino)thiazol-4-yl)-1H-pyrazole-5-carboxamide

According to Scheme 13, Step 6: A solution of 4-(2-bromoacetyl)-N-methoxy-1-(4-methoxybenzyl)-N-methyl-1H-pyrazole-3-carboxamide (9.45 mmol, 3.74 g), N-ethyl-N-isopropylpropan-2-amine (18.9 mmol, 3.23 mL) and 1-(4-methylpyrimidin-2-yl)thiourea (9.45 mmol, 1.59 g) in acetone (47 mL) was stirred at rt overnight. Some more N-ethyl-N-isopropylpropan-2-amine (2.48 mmol, 0.42 mL) was added and the reaction mixture was stirred for 3 h. Then the crude mixture was filtered and the filtrate evaporated to dryness. The resulting crude residue was purified by flash column chromatography on silica gel with cyclohexane/EtOAc (50:50 to 20:80) as eluent to yield N-methoxy-1-(4-methoxybenzyl)-N-methyl-4-(2-(4-methylpyrimidin-2-ylamino)thiazol-4-yl)-1H-pyrazole-5-carboxamide (3.41 mmol, 1.59 g, 36%).

UPLC-MS: RT=0.96 min; MS m/z ES⁺=466.

### 1-(4-Methoxybenzyl)-4-(2-(4-methylpyrimidin-2-ylamino)thiazol-4-yl)-1H-pyrazole-5-carboxylic acid

According to Scheme 13, Step 7: A solution of NaOH 3M (0.68 mL) was added slowly at rt to a solution of N-methoxy-1-(4-methoxybenzyl)-N-methyl-4-(2-(4-methylpyrimidin-2-ylamino)thiazol-4-yl)-1H-pyrazole-5-carboxamide (0.82 mmol, 380 mg) in MeOH (2 mL) and the resulting mixture was stirred, at 50° C. for 4 h. After evaporation of the solvent, the reaction mixture was partitioned between NaOH 1 M and Et₂O. The organic layer was washed with NaOH 1 M. Then the aqueous layer was acidified, the white precipitate was recovered by filtration and was washed with water to yield after evaporation 1-(4-methoxybenzyl)-4-(2-(4-methylpyrimidin-2-ylamino)thiazol-4-yl)-1H-pyrazole-5-carboxylic acid (0.82 mmol, 345 mg, 100%) as a white solid.

UPLC-MS: RT=1.00 min; MS m/z ES⁺=423.

### 1-(4-Methoxybenzyl)-N-methyl-4-(2-(4-methylpyrimidin-2-ylamino)thiazol-4-yl)-1H-pyrazole-5-carboxamide

According to Scheme 13, Step 8: A mixture of 1-(4-methoxybenzyl)-4-(2-(4-methylpyrimidin-2-ylamino)thiazol-4-yl)-1H-pyrazole-5-carboxylic acid (0.27 mmol, 116 mg), EDCI.HCl (0.33 mmol, 63 mg), methanamine hydrochloride (0.55 mmol, 37 mg) and 1H-benzo[d][1,2,3]triazol-1-ol hydrate (0.33 mmol, 50 mg) in DCM (2.8 mL) was stirred at rt overnight. The reaction mixture was diluted with water and was washed with DCM. The combined organic phases were dried over MgSO₄, filtered and evaporated. The resulting crude residue was purified by flash column chromatography on silica gel with DCM/EtOH/NH₃aq (100:0:0 to 95:4.5:0.5) as eluent to yield 1-(4-methoxybenzyl)-N-methyl-4-(2-(4-methylpyrimidin-2-ylamino)thiazol-4-yl)-1H-pyrazole-5-carboxamide (69 pmol, 30 mg, 25%) as a white solid.

UPLC-MS: RT=0.99 min; MS m/z ES⁺=436.

### N-Methyl-4-(2-(4-methylpyrimidin-2-ylamino)thiazol-4-yl)-1H-pyrazole-5-carboxamide

According to Scheme 13, Step 9: To a solution of 1-(4-methoxybenzyl)-N-methyl-4-(2-(4-methylpyrimidin-2-ylamino)thiazol-4-yl)-1H-pyrazole-5-carboxamide (69 pmol, 30 mg) in TFA (344 μl) was added trifluoromethanesulfonic acid (0.34 mmol, 30.5 μl) and the reaction mixture was stirred for 2 h at rt. The reaction mixture was diluted with DCM and was washed with water. The combined organic phases were dried over MgSO₄, filtered and evaporated. The resulting crude residue was purified by SCX2 column with EtOH then DCM/EtOH/NH₃ as eluent and by preparative. HPLC to yield N-methyl-4-(2-(4-methylpyrimidin-2-ylamino)thiazol-4-yl)-1H-pyrazole-5-carboxamide (5.1 pmol, 1.6 mg, 7%) as a white solid.

UPLC-MS: RT=0.65 min; MS m/z ES⁺=316;

¹H-NMR (CD₃OD, 300 MHz): 2.51 (3H, s), 3.05 (3H, s), 6.89 (1H, d), 7.29 (1H, s), 7.97 (1H, s), 8.45 (1H, d).

### Example 14

### 1-(2-(4-Methylpyrimidin-2-ylamino)-4-(1H-pyrazol-4-yl)thiazol-5-yl)ethanone (Final Compound 1-46) tert-Butyl 4-(1-(4-methoxybenzyl)-1H-pyrazol-4-yl)thiazol-2-ylcarbamate

According to Scheme 14, Step 1: A mixture of 4-(1-(4-methoxybenzyl)-1H-pyrazol-4-yl)thiazol-2-amine (0.87 mmol, 250 mg), pivalic anhydride (1.05 mmol, 195 mg) and DMAP (0.087 mmol, 10.7 mg) in DCM (4.4 mL) was stirred at rt overnight. Additional pivalic anhydride (0.43 mmol, 81 mg) and DMAP (0.087 mmol, 10.7 mg) were added and the mixture was stirred for another 3 h. The mixture was partitioned between DCM and water. The organic layer was dried over Na₂SO₄, filtered and concentrated to dryness. The resulting crude product was purified by flash chromatography over silica gel using cyclohexane/EtOAc (100:0 to 60:40) as eluent to afford tert-butyl 4-(1-(4-methoxybenzyl)-1H-pyrazol-4-yl)thiazol-2-ylcarbamate (0.53 mmol, 205 mg, 61%).

UPLC-MS: RT=1.06 min; MS m/z ES⁴=387.

### tert-Butyl 5-bromo-4-(1-(4-methoxybenzyl)-1H-pyrazol-4-yl)thiazol-2-ylcarbamate

According to Scheme 14, Step 2: To a mixture of tert-butyl 4-(1-(4-methoxybenzyl)-1H-pyrazol-4-yl)thiazol-2-ylcarbamate (0.53 mmol, 205 mg) in CHCl₃ (5.3 mL) was added 1-bromopyrrolidine-2,5-dione (0.58 mmol, 104 mg) at rt. The resulting mixture was stirred at it for 1 h. The mixture was dissolved in DCM and washed with water. The organic layer was dried over Na₂SO₄, filtered and concentrated to dryness to afford tert-butyl 4-(1-(4-methoxybenzyl)-1H-pyrazol-4-yl)thiazol-2-ylcarbamate (0.49 mmol, 229 mg, 93%).

UPLC-MS: RT=1.16 min.

### tert-Butyl 5-acetyl-4-(1-(4-methoxybenzyl)-1H-pyrazol-4-yl)thiazol-2-ylcarbamate

According to Scheme 14, Step 3: To a mixture of tert-butyl 5-bromo-4-(1-(4-methoxybenzyl)-1H-pyrazol-4-yl)thiazol-2-ylcarbamate (0.32 mmol, 148 mg) in DMF (3.2 mL) was added at it tributyl(1-ethoxyvinyl)stannane (636 μmol, 315 μL) and PdCl₂(dppf) (32 μmmol, 23.3 mg). The reaction mixture was stirred at 100° C. for 40 min in the microwave oven. 1M HCl was added and the mixture was stirred at it for 20 min. The mixture was partitioned between EtOAc and saturated aqueous Na₂CO₃. The organic layer was washed with water twice, dried over Na₂SO₄, filtered and concentrated to dryness. The resulting crude product was purified by flash chromatography over silica gel using cyclohexane/EtOAc (100:0 to 70:30) as eluent to afford tert-butyl-5-acetyl-4-(1-(4-methoxybenzyl)-1H-pyrazol-4-yl)thiazol-2-ylcarbamate (0.89 mmol, 38 mg, 28%) and 1-(2-amino-4-(1-(4-methoxybenzyl)-1H-pyrazol-4-yl)thiazol-5-yl)ethanone (0.83 mmol, 27 mg, 26%).

UPLC-MS: RT=1.06 min; MS m/z ES⁺=429 and RT=0.73 min; MS m/z ES⁺=329 respectively.

### 1-(2-Amino-4-(1-(4-methoxybenzyl)-1H-pyrazol-4-yl)thiazol-5-yl)ethanone

According to Scheme 14, Step 4: Further deprotected compound could be obtained by stirring tert-butyl 5-acetyl-4-(1-(4-methoxybenzyl)-1H-pyrazol-4-yl)thiazol-2-ylcarbamate (0.10 mmol, 45 mg) in the presence of 1M HCl at rt for 20 min. Purification as in Step 3 afforded 1-(2-amino-4-(1-(4-methoxybenzyl)-1H-pyrazol-4-yl)thiazol-5-yl)ethanone quantitatively.

### 1-(4-(1-(4-Methoxybenzyl)-1H-pyrazol-4-yl)-2-(4-methylpyrimidin-2-ylamino)thiazol-5-yl)ethanone

According to Scheme 14, Step 5: To a mixture of 1-(2-amino-4-(1-(4-methoxybenzyl)-1H-pyrazol-4-yl)thiazol-5-yl)ethanone (19.5 gmol, 64 mg) in dioxane (2 mL) were added 2-bromo-4-methylpyrimidine (0.24 mmol, 37 mg), (9,9-dimethyl-9H-xanthene-4,5-diyl)bis(diphenylphosphine) (0.029 mmol, 17 mg), Pd(OAc)₂ (19 gmol, 4.4 mg) and Cs₂CO₃ (0.39 mmol, 127 mg). The resulting mixture was stirred at 120° C. in the microwave oven for 30 min. The reaction mixture was concentrated to dryness. The residue was partitioned between DCM and saturated aqueous Na₂CO₃. The aqueous layer was extracted again and the combined organic layers was dried over Na₂SO₄, filtered and concentrated to dryness. The resulting crude product was purified by flash chromatography over silica gel using DCM/MeOH (100:0 to 97:3) as eluent to afford 1-(4-(1-(4-methoxybenzyl)-1H-pyrazol-4-yl)-2-(4-methylpyrimidin-2-ylamino)thiazol-5-yl)ethanone (71 gmol, 30 mg, 37%).

UPLC-MS: RT=0.99 min; MS m/z ES⁺=421.

### 1-(2-(4-Methylpyrimidin-2-ylamino)-4-(1H-pyrazol-4-yl)thiazol-5-yl)ethanone

According to Scheme 14, Step 6: To 1-(4-(1-(4-methoxybenzyl)-1H-pyrazol-4-yl)-2-(4-methylpyrimidin-2-ylamino)thiazol-5-yl)ethanone (71 gmol, 30 mg) in TFA (2 mL) was added trifluoromethanesulfonic acid (0.713 mmol, 63 μL). The reaction mixture was heated at 70° C. for 2 h. The reaction mixture was cooled to rt, neutralized with saturated aqueous Na₂CO₃ and extracted three times with DCM. The combined organic layers was dried over Na₂SO₄, filtered and concentrated to dryness. The resulting crude product was purified through SCX column (2 g) and eluted with MeOH to afford 1-(2-(4-methylpyrimidin-2-ylamino)-4-(1H-pyrazol-4-yl)thiazol-5-yl)ethanone (40 μmol, 12 mg, 56%).

UPLC-MS: RT=0.70 min; MS m/z ES⁺=301;

¹H-NMR (CD₃OD, 300 MHz) 8.48 (1H, d), 7.35 (2H, m), 6.95 (1H, d), 2.54 (3H, s), 2.52 (3H, s).

### Example 15

### 5-Ethoxy-N-(4-methylpyrimidin-2-yl)-4-(1H-pyrazol-4-yl)thiazol-2-amine (Final Compound 1-47)

### 4-(1-(4-Methoxybenzyl)-1H-pyrazol-4-yl)-N-(4-methylpyrimidin-2-yl)thiazol-2-amine

According to Scheme 15, Step 1: 2-Bromo-1-(1-(4-methoxybenzyl)-1H-pyrazol-4-yl)ethanone (3.23 mmol, 1.00 g) and 1-(4-methylpyrimidin-2-yl)thiourea (3.23 mmol, 0.544 g) were dissolved in acetone (30 mL) and the solution was heated at reflux for 1 h. The reaction mixture was cooled to rt, and then filtered to afford 4-(1-(4-methoxybenzyl)-1H-pyrazol-4-yl)-N-(4-methylpyrimidin-2-yl)thiazol-2-amine (3.17 mmol, 1.20 g, 98%).

UPLC-MS: RT=0.98 min; MS m/z ES⁺=379.

### 5-Chloro-4-(1-(4-methoxybenzyl)-1H-pyrazol-4-yl)-N-(4-methylpyrimidin-2-yl)thiazol-2-amine

According to Scheme 15, Step 2: 4-(1-(4-Methoxybenzyl)-1H-pyrazol-4-yl)-N-(4-methylpyrimidin-2-yl)thiazol-2-amine (1.58 mmol, 600 mg) and 1-chloropyrrolidine-2,5-dione (1.585 mmol, 212 mg) were dissolved in DMF (7 mL). After 1 h of stirring at rt, the mixture was partitioned between EtOAc and a saturated aqueous Na₂CO₃ solution. The organic layer was dried over MgSO₄, filtered and concentrated to dryness. The resulting crude product was purified by flash chromatography over silica gel using DCM/MeOH (100:0 to 97:3) as eluent to afford 5-chloro-4-(1-(4-methoxybenzyl)-1H-pyrazol-4-yl)-N-(4-methylpyrimidin-2-yl)thiazol-2-amine (967 μmol, 400 mg, 61%)

UPLC-MS: RT=1.16 min; MS m/z ES⁺=413.

### 5-Ethoxy-4-(1-(4-methoxybenzyl)-1H-pyrazol-4-yl)-N-(4-methylpyrimidin-2-yl)thiazol-2-amine

According to Scheme 15, Step 3: NaH (4.84 mmol, 194 mg) was reacted with EtOH (48.4 mmol, 2.23 g). After 10 min, 5-chloro-4-(1-(4-methoxybenzyl)-1H-pyrazol-4-yl)-N-(4-methylpyrimidin-2-yl)thiazol-2-amine (0.24 mmol, 100 mg) was added and the solution was stirred for 15 min at rt. The solution was then heated in a sealed tube at 120° C. for 4 h. Further excess equivalents of NaH in EtOH was added and the solution stirred for 24 h at 120° C. The reaction mixture was concentrated to dryness to afford the crude product, 5-ethoxy-4-(1-(4-methoxybenzyl)-1H-pyrazol-4-yl)-N-(4-methylpyrimidin-2-yl)thiazol-2-amine which was used without further purification.

UPLC-MS: RT=1.07 min; MS m/z ES⁺=423.

### 5-Ethoxy-N-(4-methylpyrimidin-2-yl)-4-(1H-pyrazol-4-yl)thiazol-2-amine

According to Scheme 15, Step 4: 5-Ethoxy-4-(1-(4-methoxybenzyl)-1H-pyrazol-4-yl)-N-(4-methylpyrimidin-2-yl)thiazol-2-amine (237 μmol, 100 mg) was dissolved in TFA (2 mL). The solution was stirred at rt for 8 h with temperature raised to 110° C. Excess trifluoromethanesulfonic acid (2.367 mmol, 355 mg) was added and solution was heated at for 4 h at 120° C. in a sealed tube. The reaction mixture was diluted in water and extracted with EtOAc. The organic layer was dried over MgSO₄, filtered and concentrated to dryness. The resulting crude product was purified by flash chromatography over silica gel using DCM/MeOH (100:0 to 95:5) as eluent and then by preparative HPLC to afford 5-ethoxy-N-(4-methylpyrimidin-2-yl)-4-(1H-pyrazol-4-yl)thiazol-2-amine (4 μmol, 1.5 mg, 2%).

UPLC-MS: RT=0.8 min; MS m/z ES⁺=303;

¹H-NMR (CD₃OD, 300 MHz): 8.4 (d, 1H), 8 (s, 2H), 6.9 (d, 1H), 4.2 (q, 2H), 2.5 (s, 3H), 1.45 (t, 3H).

The compounds in the following Table have been synthesized according to the same methods as previous examples 1 to 15, as denoted in the column denoted as “Exp. nr”. The compounds denoted with the asterisk have been exemplified in the Examples.

TABLE 1
Compounds prepared according to the Examples.

| Co. nr. | Exp nr. | 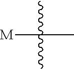 | 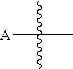 | 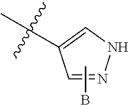 |
|---|---|---|---|---|
| 1-1 | 4* | 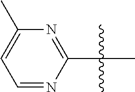 [SMILES: *c1nccc(C)n1] | H— | 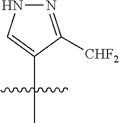 [SMILES: *c1c[nH]nc1C(F)F] |
| 1-2 | 6 | 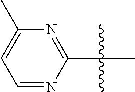 [SMILES: *c1nccc(C)n1] | H— | 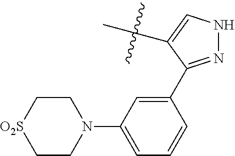 [SMILES: *c1c[nH]nc1-c1cccc(N2CCS(=O)(=O)CC2)c1] |
| 1-3 | 6 | 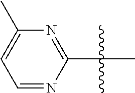 [SMILES: *c1nccc(C)n1] | H— | 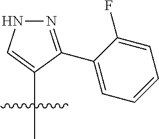 [SMILES: *c1c[nH]nc1-c1ccccc1F] |
| 1-4 | 7 | 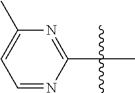 [SMILES: *c1nccc(C)n1] | H— | 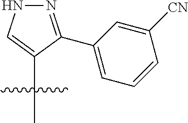 [SMILES: *c1c[nH]nc1-c1cccc(C#N)c1] |
| 1-5 | 6 | 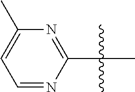 [SMILES: *c1nccc(C)n1] | H— | 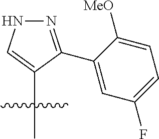 [SMILES: *c1c[nH]nc1-c1cc(F)ccc1OC] |
| 1-6 | 6 | 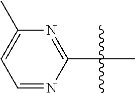 [SMILES: *c1nccc(C)n1] | H— | 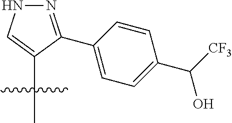 [SMILES: *c1c[nH]nc1-c1ccc(C(O)C(F)(F)F)cc1] |
| 1-7 | 6 | 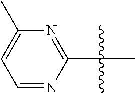 [SMILES: *c1nccc(C)n1] | H— | 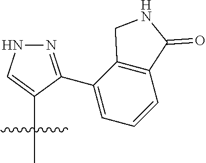 [SMILES: *c1c[nH]nc1-c1cccc2c1CNC2=O] |
| 1-8 | 6 | 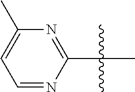 [SMILES: *c1nccc(C)n1] | H— | 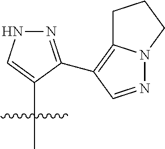 [SMILES: *c1c[nH]nc1-c1cnn2c1CCC2] |
| 1-9 | 6 | 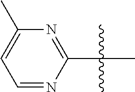 [SMILES: *c1nccc(C)n1] | H— | 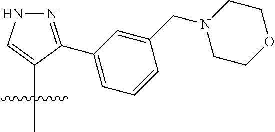 [SMILES: *c1c[nH]nc1-c1cccc(CN2CCOCC2)c1] |
| 1-10 | 6 | 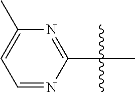 [SMILES: *c1nccc(C)n1] | H— | 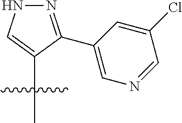 [SMILES: *c1c[nH]nc1-c1cncc(Cl)c1] |
| 1-11 | 6 | 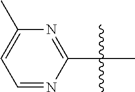 [SMILES: *c1nccc(C)n1] | H— | 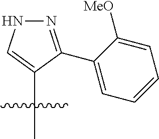 [SMILES: *c1c[nH]nc1-c1ccccc1OC] |
| 1-12 | 6 | 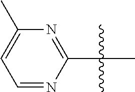 [SMILES: *c1nccc(C)n1] | H— | 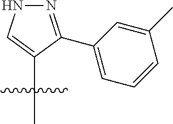 [SMILES: *c1c[nH]nc1-c1cccc(C)c1] |
| 1-13 | 10* | 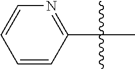 [SMILES: *c1ccccn1] | H— | 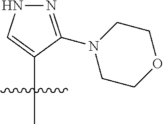 [SMILES: *c1c[nH]nc1N1CCOCC1] |
| 1-14 | 6 | 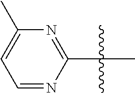 [SMILES: *c1nccc(C)n1] | H— | 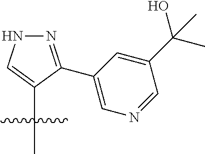 [SMILES: *c1c[nH]nc1-c1cncc(C(C)(C)O)c1] |
| 1-15 | 6 | 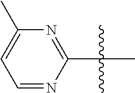 [SMILES: *c1nccc(C)n1] | H— | 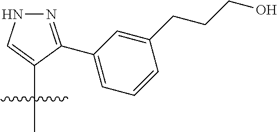 [SMILES: *c1c[nH]nc1-c1cccc(CCCO)c1] |
| 1-16 | 6 | 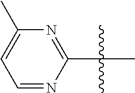 [SMILES: *c1nccc(C)n1] | H— | 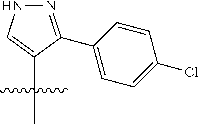 [SMILES: *c1c[nH]nc1-c1ccc(Cl)cc1] |
| 1-17 | 6 | 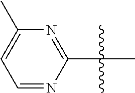 [SMILES: *c1nccc(C)n1] | H— | 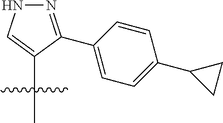 [SMILES: *c1c[nH]nc1-c1ccc(C2CC2)cc1] |
| 1-18 | 6 | 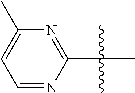 [SMILES: *c1nccc(C)n1] | H— | 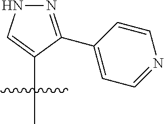 [SMILES: *c1c[nH]nc1-c1ccncc1] |
| 1-19 | 6 | 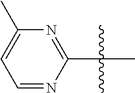 [SMILES: *c1nccc(C)n1] | H— | 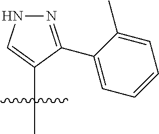 [SMILES: *c1c[nH]nc1-c1ccccc1C] |
| 1-20 | 6 | 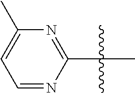 [SMILES: *c1nccc(C)n1] | H— | 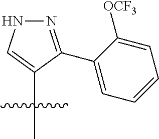 [SMILES: *c1c[nH]nc1-c1ccccc1OC(F)(F)F] |
| 1-21 | 6 | 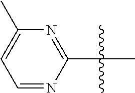 [SMILES: *c1nccc(C)n1] | H— | 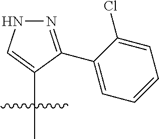 [SMILES: *c1c[nH]nc1-c1ccccc1Cl] |
| 1-22 | 6 | 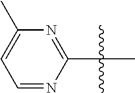 [SMILES: *c1nccc(C)n1] | H— | 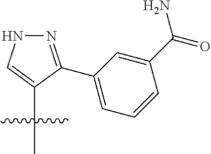 [SMILES: *c1c[nH]nc1-c1cccc(C(N)=O)c1] |
| 1-23 | 6 | 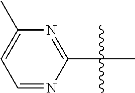 [SMILES: *c1nccc(C)n1] | H— | 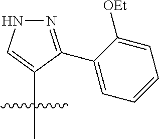 [SMILES: *c1c[nH]nc1-c1ccccc1OCC] |
| 1-24 | 6 | 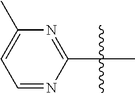 [SMILES: *c1nccc(C)n1] | H— | 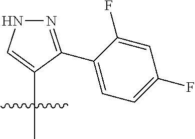 [SMILES: *c1c[nH]nc1-c1ccc(F)cc1F] |
| 1-25 | 11* | 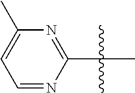 [SMILES: *c1nccc(C)n1] | H— | 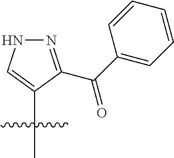 [SMILES: *c1c[nH]nc1C(=O)c1ccccc1] |
| 1-26 | 12* | 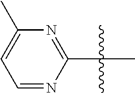 [SMILES: *c1nccc(C)n1] | H— | 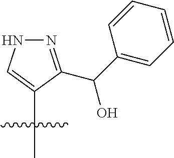 [SMILES: *c1c[nH]nc1C(O)c1ccccc1] |
| 1-27 | 11 | 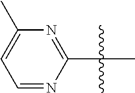 [SMILES: *c1nccc(C)n1] | H— | 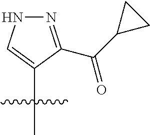 [SMILES: *c1c[nH]nc1C(=O)C1CC1] |
| 1-28 | 5* | 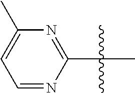 [SMILES: *c1nccc(C)n1] | H— | 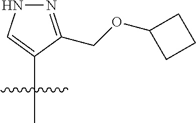 [SMILES: *c1c[nH]nc1COC1CCC1] |
| 1-29 | 2* | 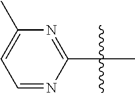 [SMILES: *c1nccc(C)n1] | H— | 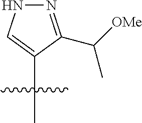 [SMILES: *c1c[nH]nc1C(C)OC] |
| 1-30 | 6 | 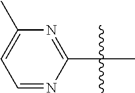 [SMILES: *c1nccc(C)n1] | H— | 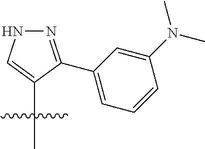 [SMILES: *c1c[nH]nc1-c1cccc(N(C)C)c1] |
| 1-31 | 6* | 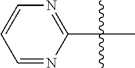 [SMILES: *c1ncccn1] | H— | 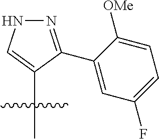 [SMILES: *c1c[nH]nc1-c1cc(F)ccc1OC] |
| 1-32 | 13* | 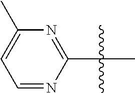 [SMILES: *c1nccc(C)n1] | H— | 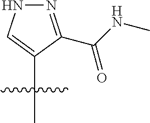 [SMILES: *c1c[nH]nc1C(=O)NC] |
| 1-33 | 9 | 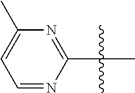 [SMILES: *c1nccc(C)n1] | H— | 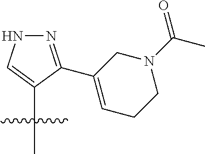 [SMILES: *c1c[nH]nc1C1=CCCN(C(C)=O)C1] |
| 1-34 | 7 | 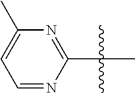 [SMILES: *c1nccc(C)n1] | H— | 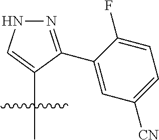 [SMILES: *c1c[nH]nc1-c1cc(C#N)ccc1F] |
| 1-35 | 7 | 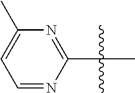 [SMILES: *c1nccc(C)n1] | H— | 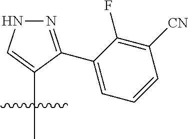 [SMILES: *c1c[nH]nc1-c1cccc(C#N)c1F] |
| 1-36 | 1* | 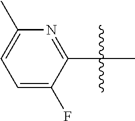 [SMILES: *c1nc(C)ccc1F] | H— | 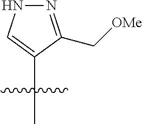 [SMILES: *c1c[nH]nc1COC] |
| 1-37 | 1 | 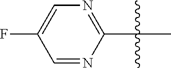 [SMILES: *c1ncc(F)cn1] | H— | 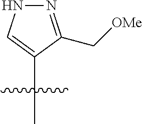 [SMILES: *c1c[nH]nc1COC] |
| 1-38 | 1 | 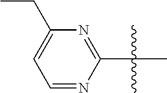 [SMILES: *c1nccc(CC)n1] | H— | 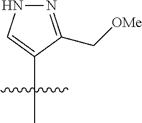 [SMILES: *c1c[nH]nc1COC] |
| 1-39 | 9* | 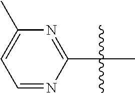 [SMILES: *c1nccc(C)n1] | H— | 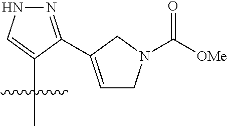 [SMILES: *c1c[nH]nc1C1=CCN(C(=O)OC)C1] |
| 1-40 | 9 | 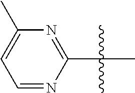 [SMILES: *c1nccc(C)n1] | H— | 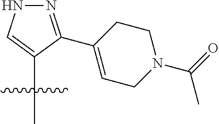 [SMILES: *c1c[nH]nc1C1=CCN(C(C)=O)CC1] |
| 1-41 | 9 | 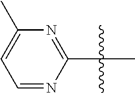 [SMILES: *c1nccc(C)n1] | H— | 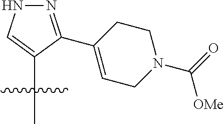 [SMILES: *c1c[nH]nc1C1=CCN(C(=O)OC)CC1] |
| 1-42 | 8* | 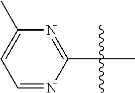 [SMILES: *c1nccc(C)n1] | H— | 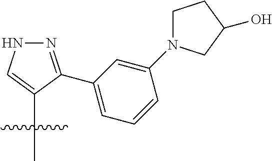 [SMILES: *c1c[nH]nc1-c1cccc(N2CCC(O)C2)c1] |
| 1-43 | 7* | 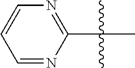 [SMILES: *c1ncccn1] | H— | 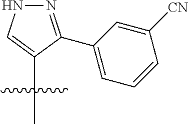 [SMILES: *c1c[nH]nc1-c1cccc(C#N)c1] |
| 1-44 | 3* | 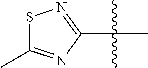 [SMILES: *c1nsc(C)n1] | NC— | 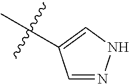 [SMILES: *c1cn[nH]c1] |
| 1-45 | 3 | 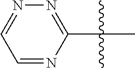 [SMILES: *c1nccnn1] | NC— | 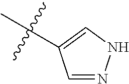 [SMILES: *c1cn[nH]c1] |
| 1-46 | 14* | 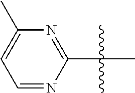 [SMILES: *c1nccc(C)n1] | MeCO— | 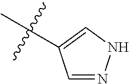 [SMILES: *c1cn[nH]c1] |
| 1-47 | 15* | 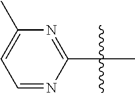 [SMILES: *c1nccc(C)n1] | EtO— | 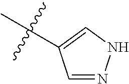 [SMILES: *c1cn[nH]c1] |

TABLE 2
Physico-chemical data for some compounds (nd = not determined).

| | M.p. | MW | | | |
|---|---|---|---|---|---|
| Co. Nr | (° C.) | (theor) | [MH⁺] | RT (min) | Method |
| 1-1 | 292 | 308.31 | 309 | 0.82 | UPLC-MS |
| 1-2 | nd | 467.57 | 468 | 2.60 | LC-MS |
| 1-3 | nd | 352.39 | 353 | 2.78 | LC-MS |
| 1-4 | nd | 359.41 | 360 | 2.67 | LC-MS |
| 1-5 | 247 | 382.41 | 383 | 2.67 | LC-MS |
| 1-6 | nd | 432.42 | 433 | 2.77 | LC-MS |
| 1-7 | nd | 389.43 | 390 | 2.46 | LC-MS |
| 1-8 | nd | 364.43 | 365 | 2.42 | LC-MS |
| 1-9 | nd | 433.53 | 434 | 2.29 | LC-MS |
| 1-10 | nd | 369.83 | 370 | 2.73 | LC-MS |
| 1-11 | nd | 364.42 | 365 | 2.54 | LC-MS |
| 1-12 | nd | 348.42 | 349 | 2.65 | LC-MS |
| 1-13 | nd | 328.39 | 329 | 0.61 | UPLC-MS |
| 1-14 | nd | 393.47 | 394 | 2.62 | LC-MS |
| 1-15 | nd | 392.48 | 393 | 2.46 | LC-MS |
| 1-16 | nd | 368.84 | 369 | 2.56 | LC-MS |
| 1-17 | nd | 374.46 | 375 | 2.65 | LC-MS |
| 1-18 | nd | 335.39 | 336 | 1.77 | LC-MS |
| 1-19 | nd | 348.42 | 349 | 2.61 | LC-MS |
| 1-20 | nd | 418.40 | 419 | 2.70 | LC-MS |
| 1-21 | nd | 368.84 | 369 | 2.50 | LC-MS |
| 1-22 | nd | 377.42 | 378 | 2.91 | LC-MS |
| 1-23 | nd | 378.45 | 379 | 2.76 | LC-MS |
| 1-24 | nd | 370.38 | 371 | 2.57 | LC-MS |
| 1-25 | nd | 362.41 | 363 | 0.90 | UPLC-MS |
| 1-26 | 249.5 | 364.42 | 365 | 0.82 | UPLC-MS |
| 1-27 | 239-240 | 326.38 | 327 | 0.81 | UPLC-MS |
| 1-28 | nd | 342.42 | 343 | 0.88 | UPLC-MS |
| 1-29 | nd | 316.38 | 317 | 0.75 | UPLC-MS |
| 1-30 | 239-241 | 377.47 | 378 | 0.79 | UPLC-MS |
| 1-31 | nd | 368.39 | 369 | 2.72 | LC-MS |
| 1-32 | nd | 315.35 | 316 | 0.65 | UPLC-MS |
| 1-33 | nd | 381.45 | 382 | 2.43 | LC-MS |
| 1-34 | nd | 377.40 | 378 | 0.84 | UPLC-MS |
| 1-35 | nd | 377.40 | 378 | 0.87 | UPLC-MS |
| 1-36 | nd | 319.36 | 320 | 0.88 | UPLC-MS |
| 1-37 | nd | 306.32 | 307 | 0.72 | UPLC-MS |
| 1-38 | nd | 316.38 | 317 | 0.82 | UPLC-MS |
| 1-39 | nd | 383.43 | 384 | 2.43 | LC-MS |
| 1-40 | nd | 381.45 | 382 | 2.44 | LC-MS |
| 1-41 | nd | 397.45 | 398 | 2.62 | LC-MS |
| 1-42 | nd | 419.50 | 420 | 2.63 | LC-MS |
| 1-43 | nd | 345.38 | 346 | 3.05 | LC-MS |
| 1-44 | nd | 289.34 | 290 | 2.62 | LC-MS |
| 1-45 | nd | 270.27 | 271 | 2.55 | LC-MS |
| 1-46 | 288-291 | 300.34 | 301 | 0.70 | UPLC-MS |
| 1-47 | nd | 302.35 | 303 | 0.80 | UPLC-MS |

### Pharmacology

The compounds provided in the present invention are positive allosteric modulators of mGluR4. As such, these compounds do not appear to bind to the orthosteric glutamate recognition site, and do not activate the mGluR4 by themselves. Instead, the response of mGluR4 to a concentration of glutamate or mGluR4 agonist is increased when compounds of Formula (I) are present. Compounds of Formula (I) are expected to have their effect at mGluR4 by virtue of their ability to enhance the function of the receptor.

mGluR4 Assay on HEK-Expressing Human mGluR4

The compounds of the present invention are positive allosteric modulators of mGluR4 receptor. Their activity was examined on recombinant human mGluR4a receptors by detecting changes in intracellular Ca²⁺ concentration, using the fluorescent Ca²⁺-sensitive dye Fluo4-(AM) and a Fluorometric Imaging Plate Reader (FLIPR, Molecular Devices, Sunnyvale, Calif.).

### Transfection and Cell Culture

The cDNA encoding the human metabotropic glutamate receptor (hmGluR4), (accession number NM_{—}000841.1, NCBI Nucleotide database browser), was subcloned into an expression vector containing also the Hygromycin resistance gene. In parallel, the cDNA encoding a G protein allowing redirection of the activation signal to intracellular calcium flux was subcloned into a different expression vector containing also the Puromycin resistance gene. Transfection of both these vectors into HEK293 cells with PolyFect reagent (Qiagen) according to supplier's protocol, and hygromycin and puromycin treatment allowed selection of antibiotic resistant cells which had integrated stably one or more copies of the plasmids. Positive cellular clones expressing hmGluR4 were identified in a functional assay measuring changes in calcium flux in response to glutamate or selective known mGluR4 orthosteric agonists and antagonists. HEK-293 cells expressing hmGluR4 were maintained in media containing DMEM, dialyzed Fetal Calf Serum (10%), Glutamax™ (2 mM), Penicillin (100 units/mL), Streptomycin (100 μg/mL), Geneticin (100 μg/mL) and Hygromycin-B (40 μg/mL) and Puromycin (1 μg/mL) at 37° C./5% CO₂.

### Fluorescent Cell Based-Ca²⁺ Mobilization Assay

Human mGluR4 HEK-293 cells were plated out 24 hours prior to FLIPR³⁸⁴ assay in black-walled, clear-bottomed, poly-L-ornithine-coated 384-well plates at a density of 25,000 cells/well in a glutamine/glutamate free DMEM medium containing foetal bovine serum (10%), penicillin (100 units/mL) and streptomycin (100 μg/mL) at 37° C./5% CO₂.

On the day of the assay, the medium was aspirated and the cells were loaded with a 3 μM solution of Fluo4-AM (LuBioScience, Lucerne, Switzerland) in 0.03% pluronic acid. After 1 hour at 37° C./5% CO₂, the non incorporated dye was removed by washing cell plate with the assay buffer and the cells were left in the dark at it for six hours before testing. All assays were performed in a pH 7.4 buffered-solution containing 20 mM HEPES, 143 mM NaCl, 6 mM KCl, 1 mM MgSO₄, 1 mM CaCl₂, 0.125 mM sulfapyrazone and 0.1% glucose.

After 10 s of basal fluorescence recording, various concentrations of the compounds of the invention were added to the cells. Changes in fluorescence levels were first monitored for 180 s in order to detect any agonist activity of the compounds. Then the cells were stimulated by an EC₂₅ glutamate concentration for an additional 110 s in order to measure enhancing activities of the compounds of the invention. EC₂₅ glutamate concentration is the concentration giving 25% of the maximal glutamate response.

The concentration-response curves of representative compounds of the present invention were generated using the Prism GraphPad software (Graph Pad Inc, San Diego, USA). The curves were fitted to a four-parameter logistic equation:

(Y=Bottom+(Top−Bottom)/(1+10̂((Log EC₅₀-X)*Hill Slope)

allowing the determination of EC₅₀ values.

The Table 3 below represents the mean EC₅₀ obtained from at least three independent experiments of selected molecules performed in duplicate.

TABLE 3
Activity data for selected compounds

| Compound no. | Ca²⁺ Flux* |
|---|---|
| 1-1 | +++ |
| 1-2 | + |
| 1-3 | +++ |
| 1-4 | +++ |
| 1-5 | ++ |
| 1-6 | + |
| 1-7 | + |
| 1-8 | + |
| 1-9 | + |
| 1-10 | + |
| 1-11 | + |
| 1-12 | + |
| 1-13 | + |
| 1-14 | + |
| 1-15 | ++ |
| 1-16 | ++ |
| 1-17 | + |
| 1-18 | + |
| 1-19 | + |
| 1-20 | + |
| 1-21 | ++ |
| 1-22 | + |
| 1-23 | + |
| 1-24 | + |
| 1-26 | + |
| 1-27 | ++ |
| 1-28 | ++ |
| 1-29 | ++ |
| 1-30 | + |
| 1-31 | +++ |
| 1-32 | + |
| 1-33 | + |
| 1-34 | ++ |
| 1-35 | +++ |
| 1-37 | + |
| 1-38 | + |
| 1-39 | ++ |
| 1-40 | + |
| 1-41 | + |
| 1-42 | ++ |
| 1-43 | +++ |
| 1-44 | +++ |
| 1-45 | ++ |
| 1-46 | ++ |
| 1-47 | +++ |

*Table legend:
(+): 1 μM < EC₅₀ < 10 μM
(++): 100 nM < EC₅₀ < 1 μM
(+++): EC₅₀ < 100 nM

The results shown in Table 3 demonstrate that the compounds described in the present invention are positive allosteric modulators of human mGluR4 receptors. These compounds do not have activity by themselves but they rather increase the functional activity and/or maximal efficacy of glutamate or mGluR4 agonist.

Thus, the positive allosteric modulators provided in the present invention are expected to increase the effectiveness of glutamate or mGluR4 agonists at mGluR4 receptor. Therefore, these positive allosteric modulators are expected to be useful for treatment of various neurological and psychiatric disorders associated with glutamate dysfunction described to be treated herein and others that can be treated by such positive allosteric modulators.

The compounds of the invention can be administered either alone, or in combination with other pharmaceutical agents effective in the treatment of conditions mentioned above.

### FORMULATION EXAMPLES

Typical examples of recipes for the formulation of the invention are as follows:

### 1. Tablets

| | | |
|---|---|---|
| Active ingredient | 5 to 50 | mg |
| Di-calcium phosphate | 20 | mg |
| Lactose | 30 | mg |
| Talcum | 10 | mg |
| Magnesium stearate | 5 | mg |
| Potato starch | ad 200 | mg |

In this Example, active ingredient can be replaced by the same amount of any of the compounds according to the present invention, in particular by the same amount of any of the exemplified compounds.

### 2. Suspension

An aqueous suspension is prepared for oral administration so that each 1 milliliter contains 1 to 5 mg of one of the active compounds, 50 mg of sodium carboxymethyl cellulose, 1 mg of sodium benzoate, 500 mg of sorbitol and water ad 1 mL.

### 3. Injectable

A parenteral composition is prepared by stirring 1.5% by weight of active ingredient of the invention in 10% by volume propylene glycol and water.

### 4. Ointment

| | | |
|---|---|---|
| Active ingredient | 5 to 1000 | mg |
| Stearyl alcohol | 3 | g |
| Lanoline | 5 | g |
| White petroleum | 15 | g |
| Water | ad 100 | g |

In this Example, active ingredient can be replaced with the same amount of any of the compounds according to the present invention, in particular by the same amount of any of the exemplified compounds.

Reasonable variations are not to be regarded as a departure from the scope of the invention. It will be obvious that the thus described invention may be varied in many ways by those skilled in the art.

1. A compound having the Formula (I) wherein: 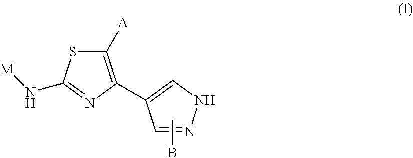
A radical is selected from the group of hydrogen, halogen, —CN, —CF₃ and an optionally substituted radical selected from the group of —(C₁-C₆)alkyl, —(C₁-C₆)haloalkyl, —(C₃-C₇)cycloalkyl, —(C₁-C₆)cyanoalkyl, —(C₁-C₆)alkylene-heteroaryl, —(C₁-C₆)alkylene-heterocycle, —(C₁-C₆)alkylene-aryl, aryl, heteroaryl, heterocycle, —(C₀-C₆)alkyl-OR¹, —O—(C₂-C₆)alkylene-OR¹, —NR¹(C₂-C₆)allylene-OR², —(C₃-C₇)cycloalkyl-(C₁-C₆)alkyl, —O—(C₃-C₇)cycloalkyl-(C₁-C₆)alkyl, —NR¹—(C₃-C₇)cycloalkyl-(C₁-C₆)alkyl, —(C₁-C₆)haloalkylene-OR¹, —(C₁-C₆)haloalkylene-NR¹R², —(C₀-C₆)alkyl-S—R¹, —O—(C₂-C₆)alkylene-S—R¹, —NR¹—(C₂-C₆)alkylene-S—R², —(C₀-C₆)alkyl-S(═O)—R¹, —O—(C₁-C₆)alkylene-S(═O)—R¹, —NR¹—(C₁-C₆)alkylene-S(═O)—R², —(C₀-C₆)alkyl-S(═O)₂—R¹, —O—(C₁-C₆)alkylene-S(═O)₂—R¹, —NR¹—(C₁-C₆)alkylene-S(═O)₂—R², —(C₀-C₆)alkyl-NR¹R², —O—(C₂-C₆)alkylene-NR¹R², —NR¹—(C₂-C₆)alkylene-NR²R³, —(C₀-C₆)alkyl-S(═O)₂NR¹R², —O—(C₁-C₆)alkylene-S(═O)₂NR¹R², —NR¹—(C₁-C₆)alkylene-S(═O)₂NR²R³, —(C₀-C₆)alkyl-NR¹—S(═O)₂R², —O—(C₂-C₆)allylene-NR¹—S(═O)₂R², —NR¹—(C₂-C₆)alkylene-NR²—S(═O)₂R³, —(C₀-C₆)alkyl-C(═O)—NR¹R², —O—(C₁-C₆)alkylene-C(═O)—NR¹R², —NR¹—(C₁-C₆)alkylene-C(═O)—NR²R³, —(C₀-C₆)alkyl-NR¹C(═O)—R², —O—(C₂-C₆)alkylene-NR¹C(═O)—R², —NR¹—(C₂-C₆)alkylene-NR²C(═O)—R³, —(C₀-C₆)alkyl-C(═O)—R¹, —O—(C₁-C₆)alkylene-C(═O)—R¹ and —NR¹—(C₁-C₆)alkylene-C(═O)—R²;
R¹, R² and R³ are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₄-C₁₀)alkylene-cycloalkyl, heteroaryl, —(C₁-C₆)alkylene-heteroaryl, aryl, heterocycle, —(C₁-C₆)alkylene-heterocycle and —(C₁-C₆)alkylene-aryl;
Any two radicals of R(R¹, R² or R³) may be taken together to form an optionally substituted 3 to 10 membered carbocyclic or heterocyclic ring;
B radical is selected from the group of hydrogen, halogen, —CN, —OH, —CF₃, —SH, —NH₂ and an optionally substituted radical selected from the group of —(C₁-C₆)alkyl, —(C₁-C₆)haloalkyl, —(C₃-C₇)cycloalkyl, —(C₁-C₆)cyanoalkyl, —(C₁-C₆)alkylene-heteroaryl, —(C₁-C₆)allylene-heterocycle, —(C₁-C₆)alkylene-aryl, aryl, heteroaryl, heterocycle, —(C₀-C₆)alkyl-OR⁴, —O—(C₂-C₆)alkylene-OR⁴, —NR⁴(C₂-C₆)alkylene-OR⁵, —(C₃-C₇)cycloalkyl-(C₁-C₆)alkyl, —O—(C₃-C₇)cycloalkyl-(C₁-C₆)alkyl, —NR⁴—(C₃-C₇)cycloalkyl-(C₁-C₆)allyl, —(C₁-C₆)haloalkylene-OR⁴, —(C₁-C₆)haloalkylene-NR⁴R⁵, —(C₀-C₆)alkyl-S—R⁴, —O—(C₂-C₆)alkylene-S—R⁴, —NR⁴—(C₂-C₆)alkylene-S—R⁵, —(C₀-C₆)alkyl-S(═O)—R⁴, —O—(C₁-C₆)alkylene-S(═O)—R⁴, —NR⁴—(C₁-C₆)alkylene-S(═O)—R⁵, —(C₀-C₆)alkyl-S(═O)₂—R⁴, —O—(C₁-C₆)alkylene-S(═O)₂—R⁴, —NR⁴—(C₁-C₆)alkylene-S(═O)₂—R⁵, —(C₀-C₆)alkyl-NR⁴R⁵, —O—(C₂-C₆)alkylene-NR⁴R⁵, —NR⁴—(C₂-C₆)alkylene-NR⁵R⁶, —(C₀-C₆)alkyl-S(═O)₂NR⁴R⁵, —O—(C₁-C₆)alkylene-S(═O)₂NR⁴R⁵, —NR⁴—(C₁-C₆)alkylene-S(═O)₂NR⁵R⁶, —(C₀-C₆)alkyl-NR⁴—S(═O)₂R⁵, —O—(C₂-C₆)alkylene-NR⁴—S(═O)₂R⁵, —NR⁴—(C₂-C₆)alkylene-NR⁵—S(═O)₂R⁶, —(C₀-C₆)alkyl-C(═O)—NR⁴R⁵, —O—(C₁-C₆)alkylene-C(═O)—NR⁴R⁵, —NR⁴—(C₁-C₆)alkylene-C(═O)—NR⁵R⁶, —(C₀-C₆)alkyl-NR⁴C(O)R⁵, —O—(C₂-C₆)alkylene-NR⁴C(═O)—R⁵, —NR⁴—(C₂-C₆)alkylene-NR⁵C(═O)—R⁶, —(C₀-C₆)alkyl-C(═O)—R⁴, —O—(C₁-C₆)alkylene-C(═O)—R⁴ and —NR⁴—(C₁-C₆)alkylene-C(═O)—R⁵;
R⁴, R⁵ and R⁶ are each independently hydrogen or an optionally substituted radical selected from the group of —(C₁-C₆)haloalkyl, —(C₁-C₆)alkyl, —(C₁-C₆)cyanoallyl, —(C₃-C₇)cycloalkyl, —(C₄-C₁₀)alkylene-cycloalkyl, heteroaryl, —(C₁-C₆)alkylene-heteroaryl, —(C₁-C₆)alkylene-heterocycle, aryl, heterocycle and —(C₁-C₆)alkylene-aryl;
Any two radicals of R (R⁴, R⁵ or R⁶) may be taken together to form an optionally substituted 3 to 10 membered carbocyclic or heterocyclic ring;
M is an optionally substituted heteroaryl;
provided that according to proviso (i) the compound is not:
4-(3-Methyl-1H-pyrazol-4-yl)-N-(pyridin-2-yl)thiazol-2-amine
N-(Pyridin-2-yl)-4-(3-(trifluoromethyl)-1H-pyrazol-4-yl)thiazol-2-amine
4-(3-Isopropyl-1H-pyrazol-4-yl)-N-(pyridin-2-yl)thiazol-2-amine
5-Methyl-4-(3-methyl-1H-pyrazol-4-yl)-N-(pyridin-2-yl)thiazol-2-amine
5-Methyl-4-(1H-pyrazol-4-yl)-N-(pyridin-2-yl)thiazol-2-amine
5-Methyl-N-(6-methylpyridin-2-yl)-4-(1H-pyrazol-4-yl)thiazol-2-amine
5-Methyl-N-(4-methylpyridin-2-yl)-4-(1H-pyrazol-4-yl)thiazol-2-amine
N-(3,5-Difluoropyridin-2-yl)-5-methyl-4-(1H-pyrazol-4-yl)thiazol-2-amine
4-(3-Methyl-1H-pyrazol-4-yl)-N-(4-methylpyridin-2-yl)thiazol-2-amine
5-Ethyl-4-(1H-pyrazol-4-yl)-N-(pyridin-2-yl)thiazol-2-amine
4-(3-Methyl-1H-pyrazol-4-yl)-N-(6-methylpyridin-2-yl)thiazol-2-amine
N-(5-Fluoropyridin-2-yl)-5-methyl-4-(1H-pyrazol-4-yl)thiazol-2-amine
4-(1H-Pyrazol-4-yl)-N-(pyridin-2-yl)thiazol-2-amine
N-(6-Chloropyridin-2-yl)-5-methyl-4-(1H-pyrazol-4-yl)thiazol-2-amine
5-Chloro-4-(1H-pyrazol-4-yl)-N-(pyridin-2-yl)thiazol-2-amine
N-(6-Fluoropyridin-2-yl)-5-methyl-4-(1H-pyrazol-4-yl)thiazol-2-amine
5-Methyl-N-(pyrazin-2-yl)-4-(1H-pyrazol-4-yl)thiazol-2-amine
N-(3-Fluoropyridin-2-yl)-5-methyl-4-(1H-pyrazol-4-yl)thiazol-2-amine
4-(1H-Pyrazol-4-yl)-2-(pyridin-2-ylamino)thiazol-5-carbonitrile
N-(6-Ethylpyridin-2-yl)-5-methyl-4-(1H-pyrazol-4-yl)thiazol-2-amine
N-(6-Chloropyridin-2-yl)-4-(1H-pyrazol-4-yl)thiazol-2-amine
N-(6-Fluoropyridin-2-yl)-4-(1H-pyrazol-4-yl)thiazol-2-amine
5-Chloro-N-(6-methylpyridin-2-yl)-4-(1H-pyrazol-4-yl)thiazol-2-amine
N-(6-Methylpyridin-2-yl)-4-(1H-pyrazol-4-yl)thiazol-2-amine
4-(1H-Pyrazol-4-yl)-N-(pyridin-2-yl)-5-(trifluoromethyl)thiazol-2-amine
5-Phenyl-4-(1H-pyrazol-4-yl)-N-(pyridin-2-yl)thiazol-2-amine
5-Fluoro-4-(1H-pyrazol-4-yl)-N-(pyridin-2-yl)thiazol-2-amine
(4-(1H-Pyrazol-4-yl)-2-(pyridin-2-ylamino)thiazol-5-yl)methanol
N-(6-Methoxypyridin-2-yl)-5-methyl-4-(1H-pyrazol-4-yl)thiazol-2-amine
4-(3-Cyclopropyl-1H-pyrazol-4-yl)-N-(pyridin-2-yl)thiazol-2-amine and
4-(3-Ethyl-1H-pyrazol-4-yl)-N-(pyridin-2-yl)thiazol-2-amine;
provided that according to proviso (ii) the compound is not:
6-(5-Methyl-4-(1H-pyrazol-4-yl)thiazol-2-ylamino)picolinonitrile
5-Morpholino-4-(1H-pyrazol-4-yl)-N-(pyridin-2-yl)thiazol-2-amine
2-(5-Methyl-4-(1H-pyrazol-4-yl)thiazol-2-ylamino)nicotinonitrile
5-(Piperidin-1-yl)-4-(1H-pyrazol-4-yl)-N-(pyridin-2-yl)thiazol-2-amine
4-(3-(Furan-2-yl)-1H-pyrazol-4-yl)-N-(pyridin-2-yl)thiazol-2-amine
4-(3-Isobutyl-1H-pyrazol-4-yl)-N-(pyridin-2-yl)thiazol-2-amine
4-(1H-Pyrazol-4-yl)-N-(pyridin-2-yl)-5-(pyrrolidin-1-yl)thiazol-2-amine
5-Fluoro-N-(6-fluoropyridin-2-yl)-4-(1H-pyrazol-4-yl)thiazol-2-amine
5-Fluoro-N-(6-methylpyridin-2-yl)-4-(1H-pyrazol-4-yl)thiazol-2-amine
N⁵,N⁵-Dimethyl-4-(1H-pyrazol-4-yl)-N²-(pyridin-2-yl)thiazole-2,5-diamine
N-(6-Chloropyridin-2-yl)-5-fluoro-4-(1H-pyrazol-4-yl)thiazol-2-amine
N-(6-Iodopyridin-2-yl)-5-methyl-4-(1H-pyrazol-4-yl)thiazol-2-amine
N-(3-Iodopyridin-2-yl)-5-methyl-4-(1H-pyrazol-4-yl)thiazol-2-amine
5-Methyl-4-(1H-pyrazol-4-yl)-N-(pyrimidin-2-yl)thiazol-2-amine
4-(5-Chloro-1H-pyrazol-4-yl)-N-(pyridin-2-yl)thiazol-2-amine
5-(Methoxymethyl)-4-(1H-pyrazol-4-yl)-N-(pyridin-2-yl)thiazol-2-amine
N-(4-Fluoropyridin-2-yl)-5-methyl-4-(1H-pyrazol-4-yl)thiazol-2-amine
5-((Diethylamino)methyl)-4-(1H-pyrazol-4-yl)-N-(pyridin-2-yl)thiazol-2-amine
5-(Morpholinomethyl)-4-(1H-pyrazol-4-yl)-N-(pyridin-2-yl)thiazol-2-amine
5-(Ethoxymethyl)-4-(1H-pyrazol-4-yl)-N-(pyridin-2-yl)thiazol-2-amine
N-(3-Fluoro-6-methylpyridin-2-yl)-5-methyl-4-(1H-pyrazol-4-yl)thiazol-2-amine
N-(5-Methoxypyridin-2-yl)-5-methyl-4-(1H-pyrazol-4-yl)thiazol-2-amine
5-Fluoro-N-(pyrazin-2-yl)-4-(1H-pyrazol-4-yl)thiazol-2-amine
4-(2-(Pyridin-2-ylamino)thiazol-4-yl)-1H-pyrazole-5-carbonitrile
N-(1-Methyl-1H-pyrazol-3-yl)-4-(1H-pyrazol-4-yl)thiazol-2-amine
5-Fluoro-N-(5-fluoropyridin-2-yl)-4-(1H-pyrazol-4-yl)thiazol-2-amine
N-(Pyridin-2-yl)-4-(3-(2,2,2-trifluoroethyl)-1H-pyrazol-4-yl)thiazol-2-amine
N-(4-Methylpyrimidin-2-yl)-4-(1H-pyrazol-4-yl)thiazol-2-amine
5-Methyl-N-(4-methylpyrimidin-2-yl)-4-(1H-pyrazol-4-yl)thiazol-2-amine
5-Methyl-N-(5-methylpyrimidin-2-yl)-4-(1H-pyrazol-4-yl)thiazol-2-amine
4-(3-Fluoro-1H-pyrazol-4-yl)-N-(pyridin-2-yl)thiazol-2-amine
4-(3-Phenyl-1H-pyrazol-4-yl)-N-(pyridin-2-yl)thiazol-2-amine
5-Methyl-4-(1H-pyrazol-4-yl)-N-(pyrimidin-4-yl)thiazol-2-amine
4-(3-(Phenylsulfonyl)-1H-pyrazol-4-yl)-N-(pyridin-2-yl)thiazol-2-amine
4-(3-(Methoxymethyl)-1H-pyrazol-4-yl)-N-(pyridin-2-yl)thiazol-2-amine
N-(2-Methylthiazol-4-yl)-4-(1H-pyrazol-4-yl)thiazol-2-amine
4-(1H-Pyrazol-4-yl)-N-(thiazol-2-yl)thiazol-2-amine
N-(6-(Fluoromethyl)pyridin-2-yl)-5-methyl-4-(1H-pyrazol-4-yl)thiazol-2-amine
N-(6-(Difluoromethyl)pyridin-2-yl)-5-methyl-4-(1H-pyrazol-4-yl)thiazol-2-amine
5-Chloro-4-(1H-pyrazol-4-yl)-N-(pyrimidin-2-yl)thiazol-2-amine
N-(4-Ethylpyrimidin-2-yl)-5-methyl-4-(1H-pyrazol-4-yl)thiazol-2-amine
N-(5-Fluoropyrimidin-2-yl)-5-methyl-4-(1H-pyrazol-4-yl)thiazol-2-amine
4-(5-(Dimethylamino)-1H-pyrazol-4-yl)-N-(pyridin-2-yl)thiazol-2-amine
5-Chloro-N-(4-methylpyrimidin-2-yl)-4-(1H-pyrazol-4-yl)thiazol-2-amine
4-(1H-Pyrazol-4-yl)-N-(pyrimidin-2-yl)-5-(trifluoromethyl)thiazol-2-amine N-(4-Isopropylpyrimidin-2-yl)-5-methyl-4-(1H-pyrazol-4-yl)thiazol-2-amine
N-(4-Methoxypyrimidin-2-yl)-4-(1H-pyrazol-4-yl)thiazol-2-amine
1-(4-(2-(4-Methylpyrimidin-2-ylamino)thiazol-4-yl)-1H-pyrazol-5-yl)ethanone
N-(5-Fluoropyrimidin-2-yl)-4-(1H-pyrazol-4-yl)thiazol-2-amine
4-(5-(Dimethylamino)-1H-pyrazol-4-yl)-N-(4-methylpyrimidin-2-yl)thiazol-2-amine
N-(4-Methylpyrimidin-2-yl)-4-(3-(piperidin-1-yl)-1H-pyrazol-4-yl)thiazol-2-amine
N-(4-Methylpyrimidin-2-yl)-4-(1H-pyrazol-4-yl)-5-(trifluoromethyl)thiazol-2-amine
4-(1H-Pyrazol-4-yl)-N-(pyrimidin-2-yl)thiazol-2-amine
N-(4-Methylpyrimidin-2-yl)-4-(3-morpholino-1H-pyrazol-4-yl)thiazol-2-amine
N-(4-Cyclopropylpyrimidin-2-yl)-5-methyl-4-(1H-pyrazol-4-yl)thiazol-2-amine
4-(5-(Diethylamino)-1H-pyrazol-4-yl)-N-(4-methylpyrimidin-2-yl)thiazol-2-amine
4-(5-(Dimethylamino)-1H-pyrazol-4-yl)-N-(pyrimidin-2-yl)thiazol-2-amine
Cyclopropyl(4-(2-(pyrimidin-2-ylamino)thiazol-4-yl)-1H-pyrazol-5-yl)methanone
1-(4-(2-(Pyrimidin-2-ylamino)thiazol-4-yl)-1H-pyrazol-5-yl)ethanone
N⁵-(2-Methoxyethyl)-N⁵-methyl-4-(1H-pyrazol-4-yl)-N²-(pyrimidin-2-yl)thiazole-2,5-diamine
N-(5-Fluoro-4-methylpyrimidin-2-yl)-4-(1H-pyrazol-4-yl)thiazol-2-amine
N-(5-Fluoro-4-methylpyrimidin-2-yl)-5-methyl-4-(1H-pyrazol-4-yl)thiazol-2-amine
4-(3-(Ethyl(methyl)amino)-1H-pyrazol-4-yl)-N-(pyrimidin-2-yl)thiazol-2-amine
4-(3-(Methylamino)-1H-pyrazol-4-yl)-N-(pyrimidin-2-yl)thiazol-2-amine
4-(5-(4-Fluorophenyl)-1H-pyrazol-4-yl)-N-(pyrimidin-2-yl)thiazol-2-amine
4-(5-(Methoxymethyl)-1H-pyrazol-4-yl)-N-(pyrimidin-2-yl)thiazol-2-amine
5-Cyclobutyl-4-(1H-pyrazol-4-yl)-N-(pyrimidin-2-yl)thiazol-2-amine
N-(Pyrimidin-2-yl)-4-(3-(pyrrolidin-1-yl)-1H-pyrazol-4-yl)thiazol-2-amine
4-(3-((2-Methoxyethyl)(methyl)amino)-1H-pyrazol-4-yl)-N-(pyrimidin-2-yl)thiazol-2-amine
N-(4-Methylpyrimidin-2-yl)-4-(3-(pyrrolidin-1-yl)-1H-pyrazol-4-yl)thiazol-2-amine
N-(4-Methylpyrimidin-2-yl)-4-(3-phenyl-1H-pyrazol-4-yl)thiazol-2-amine
(4-(2-(4-Methylpyrimidin-2-ylamino)thiazol-4-yl)-1H-pyrazol-3-yl)methanol
4-(3-(Methoxymethyl)-1H-pyrazol-4-yl)-N-(4-methylpyrimidin-2-yl)thiazol-2-amine
4-(3-(Ethylamino)-1H-pyrazol-4-yl)-N-(4-methylpyrimidin-2-yl)thiazol-2-amine and
4-(3-(2-Methoxyethyl)-1H-pyrazol-4-yl)-N-(4-methylpyrimidin-2-yl)thiazol-2-amine.

2. A compound according to claim 1 wherein:
A radical is selected from the group of hydrogen, halogen, —CN, —CF₃, and an optionally substituted radical selected from the group of —(C₁-C₆)alkyl, —(C₁-C₆)haloalkyl, —(C₃-C₇)cycloalkyl, —(C₁-C₆)cyanoalkyl, heterocycle, heteroaryl, aryl, —(C₀-C₆)alkyl-OR¹, —NR¹(C₂-C₆)alkylene-OR² and —(C₀-C₆)alkyl-NR¹R²;
R¹ and R² are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₄-C₁₀)alkylene-cycloalkyl, heteroaryl, —(C₁-C₆)alkylene-heteroaryl, aryl, heterocycle, —(C₁-C₆)allylene-heterocycle and —(C₁-C₆)alkylene-aryl;
Any two radicals of R(R¹ or R²) may be taken together to form an optionally substituted 3 to 10 membered carbocyclic or heterocyclic ring;
B radical is selected from the group of hydrogen, halogen, —CN, —CF₃, and an optionally substituted radical selected from the group of —(C₁-C₆)alkyl, —(C₁-C₆)haloalkyl, —(C₃-C₇)cycloalkyl, aryl, —(C₁-C₆)alkylene-aryl, heterocycle, —(C₀-C₆)alkyl-OR⁴, —NR⁴(C₂-C₆)alkylene-OR⁵, —(C₀-C₆)alkyl-NR⁴R⁵, —C(═O)—NR⁴R⁵, —(C₀-C6)alkyl-S(═O)₂—R⁴, —(C₀-C₆)alkyl-C(═O)—R⁴, heteroaryl and aryl-(C₁-C₆)alkylene-heterocycle;
R⁴ and R⁵ are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₄-C₁₀)alkylene-cycloalkyl, heteroaryl, —(C₁-C₆)alkylene-heteroaryl, aryl, heterocycle, —(C₁-C₆)alkylene-heterocycle and —(C₁-C₆)alkylene-aryl; and,
Any two radicals of R (R⁴ or R⁵) may be taken together to form an optionally substituted 3 to 10 membered carbocyclic or heterocyclic ring;
M is an optionally substituted heteroaryl;
provided that according to proviso (i) the compound is not:
4-(3-Methyl-1H-pyrazol-4-yl)-N-(pyridin-2-yl)thiazol-2-amine
N-(Pyridin-2-yl)-4-(3-(trifluoromethyl)-1H-pyrazol-4-yl)thiazol-2-amine
4-(3-Isopropyl-1H-pyrazol-4-yl)-N-(pyridin-2-yl)thiazol-2-amine
5-Methyl-4-(3-methyl-1H-pyrazol-4-yl)-N-(pyridin-2-yl)thiazol-2-amine
5-Methyl-4-(1H-pyrazol-4-yl)-N-(pyridin-2-yl)thiazol-2-amine
5-Methyl-N-(6-methylpyridin-2-yl)-4-(1H-pyrazol-4-yl)thiazol-2-amine
5-Methyl-N-(4-methylpyridin-2-yl)-4-(1H-pyrazol-4-yl)thiazol-2-amine
N-(3,5-Difluoropyridin-2-yl)-5-methyl-4-(1H-pyrazol-4-yl)thiazol-2-amine
4-(3-Methyl-1H-pyrazol-4-yl)-N-(4-methylpyridin-2-yl)thiazol-2-amine
5-Ethyl-4-(1H-pyrazol-4-yl)-N-(pyridin-2-yl)thiazol-2-amine
4-(3-Methyl-1H-pyrazol-4-yl)-N-(6-methylpyridin-2-yl)thiazol-2-amine
N-(5-Fluoropyridin-2-yl)-5-methyl-4-(1H-pyrazol-4-yl)thiazol-2-amine
4-(1H-Pyrazol-4-yl)-N-(pyridin-2-yl)thiazol-2-amine
N-(6-Chloropyridin-2-yl)-5-methyl-4-(1H-pyrazol-4-yl)thiazol-2-amine
5-Chloro-4-(1H-pyrazol-4-yl)-N-(pyridin-2-yl)thiazol-2-amine
N-(6-Fluoropyridin-2-yl)-5-methyl-4-(1H-pyrazol-4-yl)thiazol-2-amine
5-Methyl-N-(pyrazin-2-yl)-4-(1H-pyrazol-4-yl)thiazol-2-amine
N-(3-Fluoropyridin-2-yl)-5-methyl-4-(1H-pyrazol-4-yl)thiazol-2-amine
4-(1H-Pyrazol-4-yl)-2-(pyridin-2-ylamino)thiazol-5-carbonitrile
N-(6-Ethylpyridin-2-yl)-5-methyl-4-(1H-pyrazol-4-yl)thiazol-2-amine
N-(6-Chloropyridin-2-yl)-4-(1H-pyrazol-4-yl)thiazol-2-amine
N-(6-Fluoropyridin-2-yl)-4-(1H-pyrazol-4-yl)thiazol-2-amine
5-Chloro-N-(6-methylpyridin-2-yl)-4-(1H-pyrazol-4-yl)thiazol-2-amine
N-(6-Methylpyridin-2-yl)-4-(1H-pyrazol-4-yl)thiazol-2-amine
4-(1H-Pyrazol-4-yl)-N-(pyridin-2-yl)-5-(trifluoromethyl)thiazol-2-amine
5-Phenyl-4-(1H-pyrazol-4-yl)-N-(pyridin-2-yl)thiazol-2-amine
5-Fluoro-4-(1H-pyrazol-4-yl)-N-(pyridin-2-yl)thiazol-2-amine
(4-(1H-Pyrazol-4-yl)-2-(pyridin-2-ylamino)thiazol-5-yl)methanol
N-(6-Methoxypyridin-2-yl)-5-methyl-4-(1H-pyrazol-4-yl)thiazol-2-amine
4-(3-Cyclopropyl-1H-pyrazol-4-yl)-N-(pyridin-2-yl)thiazol-2-amine and
4-(3-Ethyl-1H-pyrazol-4-yl)-N-(pyridin-2-yl)thiazol-2-amine;
provided that according to proviso (iii) the compound is not:
6-(5-Methyl-4-(1H-pyrazol-4-yl)thiazol-2-ylamino)picolinonitrile
5-Morpholino-4-(1H-pyrazol-4-yl)-N-(pyridin-2-yl)thiazol-2-amine
2-(5-Methyl-4-(1H-pyrazol-4-yl)thiazol-2-ylamino)nicotinonitrile
5-(piperidin-1-yl)-4-(1H-pyrazol-4-yl)-N-(pyridin-2-yl)thiazol-2-amine
4-(3-(Furan-2-yl)-1H-pyrazol-4-yl)-N-(pyridin-2-yl)thiazol-2-amine
4-(3-Isobutyl-1H-pyrazol-4-yl)-N-(pyridin-2-yl)thiazol-2-amine
4-(1H-Pyrazol-4-yl)-N-(pyridin-2-yl)-5-(pyrrolidin-1-yl)thiazol-2-amine
5-Fluoro-N-(6-fluoropyridin-2-yl)-4-(1H-pyrazol-4-yl)thiazol-2-amine
5-Fluoro-N-(6-methylpyridin-2-yl)-4-(1H-pyrazol-4-yl)thiazol-2-amine
N⁵,N⁵-Dimethyl-4-(1H-pyrazol-4-yl)-N²-(pyridin-2-yl)thiazole-2,5-diamine
N-(6-Chloropyridin-2-yl)-5-fluoro-4-(1H-pyrazol-4-yl)thiazol-2-amine
N-(6-Iodopyridin-2-yl)-5-methyl-4-(1H-pyrazol-4-yl)thiazol-2-amine
N-(3-Iodopyridin-2-yl)-5-methyl-4-(1H-pyrazol-4-yl)thiazol-2-amine
5-Methyl-4-(1H-pyrazol-4-yl)-N-(pyrimidin-2-yl)thiazol-2-amine
4-(5-Chloro-1H-pyrazol-4-yl)-N-(pyridin-2-yl)thiazol-2-amine
5-(Methoxymethyl)-4-(1H-pyrazol-4-yl)-N-(pyridin-2-yl)thiazol-2-amine
N-(4-Fluoropyridin-2-yl)-5-methyl-4-(1H-pyrazol-4-yl)thiazol-2-amine
5-((Diethylamino)methyl)-4-(1H-pyrazol-4-yl)-N-(pyridin-2-yl)thiazol-2-amine
5-(Ethoxymethyl)-4-(1H-pyrazol-4-yl)-N-(pyridin-2-yl)thiazol-2-amine
N-(3-Fluoro-6-methylpyridin-2-yl)-5-methyl-4-(1H-pyrazol-4-yl)thiazol-2-amine
N-(5-Methoxypyridin-2-yl)-5-methyl-4-(1H-pyrazol-4-yl)thiazol-2-amine
5-Fluoro-N-(pyrazin-2-yl)-4-(1H-pyrazol-4-yl)thiazol-2-amine
4-(2-(Pyridin-2-ylamino)thiazol-4-yl)-1H-pyrazole-5-carbonitrile
N-(1-Methyl-1H-pyrazol-3-yl)-4-(1H-pyrazol-4-yl)thiazol-2-amine
5-Fluoro-N-(5-fluoropyridin-2-yl)-4-(1H-pyrazol-4-yl)thiazol-2-amine
N-(Pyridin-2-yl)-4-(3-(2,2,2-trifluoroethyl)-1H-pyrazol-4-yl)thiazol-2-amine
N-(4-Methylpyrimidin-2-yl)-4-(1H-pyrazol-4-yl)thiazol-2-amine
5-Methyl-N-(4-methylpyrimidin-2-yl)-4-(1H-pyrazol-4-yl)thiazol-2-amine
5-Methyl-N-(5-methylpyrimidin-2-yl)-4-(1H-pyrazol-4-yl)thiazol-2-amine
4-(3-Fluoro-1H-pyrazol-4-yl)-N-(pyridin-2-yl)thiazol-2-amine
4-(3-Phenyl-1H-pyrazol-4-yl)-N-(pyridin-2-yl)thiazol-2-amine
5-Methyl-4-(1H-pyrazol-4-yl)-N-(pyrimidin-4-yl)thiazol-2-amine
4-(3-(Phenylsulfonyl)-1H-pyrazol-4-yl)-N-(pyridin-2-yl)thiazol-2-amine
4-(3-(Methoxymethyl)-1H-pyrazol-4-yl)-N-(pyridin-2-yl)thiazol-2-amine
N-(2-Methylthiazol-4-yl)-4-(1H-pyrazol-4-yl)thiazol-2-amine
4-(1H-Pyrazol-4-yl)-N-(thiazol-2-yl)thiazol-2-amine
N-(6-(Fluoromethyl)pyridin-2-yl)-5-methyl-4-(1H-pyrazol-4-yl)thiazol-2-amine
N-(6-(Difluoromethyl)pyridin-2-yl)-5-methyl-4-(1H-pyrazol-4-yl)thiazol-2-amine
5-Chloro-4-(1H-pyrazol-4-yl)-N-(pyrimidin-2-yl)thiazol-2-amine
N-(4-Ethylpyrimidin-2-yl)-5-methyl-4-(1H-pyrazol-4-yl)thiazol-2-amine
N-(5-Fluoropyrimidin-2-yl)-5-methyl-4-(1H-pyrazol-4-yl)thiazol-2-amine
4-(5-(Dimethylamino)-1H-pyrazol-4-yl)-N-(pyridin-2-yl)thiazol-2-amine
5-Chloro-N-(4-methylpyrimidin-2-yl)-4-(1H-pyrazol-4-yl)thiazol-2-amine
4-(1H-Pyrazol-4-yl)-N-(pyrimidin-2-yl)-5-(trifluoromethyl)thiazol-2-amine
N-(4-Isopropylpyrimidin-2-yl)-5-methyl-4-(1H-pyrazol-4-yl)thiazol-2-amine
N-(4-Methoxypyrimidin-2-yl)-4-(1H-pyrazol-4-yl)thiazol-2-amine
1-(4-(2-(4-Methylpyrimidin-2-ylamino)thiazol-4-yl)-1H-pyrazol-5-yl)ethanone
N-(5-Fluoropyrimidin-2-yl)-4-(1H-pyrazol-4-yl)thiazol-2-amine
4-(5-(Dimethylamino)-1H-pyrazol-4-yl)-N-(4-methylpyrimidin-2-yl)thiazol-2-amine
N-(4-Methylpyrimidin-2-yl)-4-(3-(piperidin-1-yl)-1H-pyrazol-4-yl)thiazol-2-amine
N-(4-Methylpyrimidin-2-yl)-4-(1H-pyrazol-4-yl)-5-(trifluoromethyl)thiazol-2-amine
4-(1H-Pyrazol-4-yl)-N-(pyrimidin-2-yl)thiazol-2-amine
N-(4-Methylpyrimidin-2-yl)-4-(3-morpholino-1H-pyrazol-4-yl)thiazol-2-amine
N-(4-Cyclopropylpyrimidin-2-yl)-5-methyl-4-(1H-pyrazol-4-yl)thiazol-2-amine
4-(5-(Diethylamino)-1H-pyrazol-4-yl)-N-(4-methylpyrimidin-2-yl)thiazol-2-amine
4-(5-(Dimethylamino)-1H-pyrazol-4-yl)-N-(pyrimidin-2-yl)thiazol-2-amine
Cyclopropyl(4-(2-(pyrimidin-2-ylamino)thiazol-4-yl)-1H-pyrazol-5-yl)methanone
1-(4-(2-(Pyrimidin-2-ylamino)thiazol-4-yl)-1H-pyrazol-5-yl)ethanone
N⁵-(2-Methoxyethyl)-N⁵-methyl-4-(1H-pyrazol-4-yl)-N²-(pyrimidin-2-yl)thiazole-2,5-diamine
N-(5-Fluoro-4-methylpyrimidin-2-yl)-4-(1H-pyrazol-4-yl)thiazol-2-amine
N-(5-Fluoro-4-methylpyrimidin-2-yl)-5-methyl-4-(1H-pyrazol-4-yl)thiazol-2-amine
4-(3-(Ethyl(methyl)amino)-1H-pyrazol-4-yl)-N-(pyrimidin-2-yl)thiazol-2-amine
4-(3-(Methylamino)-1H-pyrazol-4-yl)-N-(pyrimidin-2-yl)thiazol-2-amine
4-(5-(4-Fluorophenyl)-1H-pyrazol-4-yl)-N-(pyrimidin-2-yl)thiazol-2-amine
4-(5-(Methoxymethyl)-1H-pyrazol-4-yl)-N-(pyrimidin-2-yl)thiazol-2-amine
5-Cyclobutyl-4-(1H-pyrazol-4-yl)-N-(pyrimidin-2-yl)thiazol-2-amine
N-(Pyrimidin-2-yl)-4-(3-(pyrrolidin-1-yl)-1H-pyrazol-4-yl)thiazol-2-amine
4-(3-((2-Methoxyethyl)(methyl)amino)-1H-pyrazol-4-yl)-N-(pyrimidin-2-yl)thiazol-2-amine
N-(4-Methylpyrimidin-2-yl)-4-(3-(pyrrolidin-1-yl)-1H-pyrazol-4-yl)thiazol-2-amine
N-(4-Methylpyrimidin-2-yl)-4-(3-phenyl-1H-pyrazol-4-yl)thiazol-2-amine
(4-(2-(4-Methylpyrimidin-2-ylamino)thiazol-4-yl)-1H-pyrazol-3-yl)methanol
4-(3-(Methoxymethyl)-1H-pyrazol-4-yl)-N-(4-methylpyrimidin-2-yl)thiazol-2-amine
4-(3-(Ethylamino)-1H-pyrazol-4-yl)-N-(4-methylpyrimidin-2-yl)thiazol-2-amine and
4-(3-(2-Methoxyethyl)-1H-pyrazol-4-yl)-N-(4-methylpyrimidin-2-yl)thiazol-2-amine.

3. A compound according to claim 2 having the Formula (I) wherein:
M is an optionally substituted pyrimidinyl;
provided that according to proviso (iv) the compound is not:
5-Methyl-441H-pyrazol-4-yl)-N-(pyrimidin-2-yl)thiazol-2-amine
N-(4-Methylpyrimidin-2-yl)-4-(1H-pyrazol-4-yl)thiazol-2-amine
5-Methyl-N-(4-methylpyrimidin-2-yl)-4-(1H-pyrazol-4-yl)thiazol-2-amine
5-Methyl-N-(5-methylpyrimidin-2-yl)-4-(1H-pyrazol-4-yl)thiazol-2-amine
5-Methyl-4-(1H-pyrazol-4-yl)-N-(pyrimidin-4-yl)thiazol-2-amine
5-Chloro-4-(1H-pyrazol-4-yl)-N-(pyrimidin-2-yl)thiazol-2-amine
N-(4-Ethylpyrimidin-2-yl)-5-methyl-4-(1H-pyrazol-4-yl)thiazol-2-amine
N-(5-Fluoropyrimidin-2-yl)-5-methyl-4-(1H-pyrazol-4-yl)thiazol-2-amine
5-Chloro-N-(4-methylpyrimidin-2-yl)-4-(1H-pyrazol-4-yl)thiazol-2-amine
4-(1H-Pyrazol-4-yl)-N-(pyrimidin-2-yl)-5-(trifluoromethyl)thiazol-2-amine
N-(4-Isopropylpyrimidin-2-yl)-5-methyl-4-(1H-pyrazol-4-yl)thiazol-2-amine
N-(4-Methoxypyrimidin-2-yl)-4-(1H-pyrazol-4-yl)thiazol-2-amine
1-(4-(2-(4-Methylpyrimidin-2-ylamino)thiazol-4-yl)-1H-pyrazol-5-yl)ethanone
N-(5-Fluoropyrimidin-2-yl)-4-(1H-pyrazol-4-yl)thiazol-2-amine
4-(5-(Dimethylamino)-1H-pyrazol-4-yl)-N-(4-methylpyrimidin-2-yl)thiazol-2-amine
N-(4-Methylpyrimidin-2-yl)-4-(3-(piperidin-1-yl)-1H-pyrazol-4-yl)thiazol-2-amine
N-(4-Methylpyrimidin-2-yl)-4-(1H-pyrazol-4-yl)-5-(trifluoromethyl)thiazol-2-amine
4-(1H-Pyrazol-4-yl)-N-(pyrimidin-2-yl)thiazol-2-amine
N-(4-Methylpyrimidin-2-yl)-4-(3-morpholino-1H-pyrazol-4-yl)thiazol-2-amine
N-(4-Cyclopropylpyrimidin-2-yl)-5-methyl-4-(1H-pyrazol-4-yl)thiazol-2-amine
4-(5-(Diethylamino)-1H-pyrazol-4-yl)-N-(4-methylpyrimidin-2-yl)thiazol-2-amine
4-(5-(Dimethylamino)-1H-pyrazol-4-yl)-N-(pyrimidin-2-yl)thiazol-2-amine
Cyclopropyl(4-(2-(pyrimidin-2-ylamino)thiazol-4-yl)-1H-pyrazol-5-yl)methanone
1-(4-(2-(Pyrimidin-2-ylamino)thiazol-4-yl)-1H-pyrazol-5-yl)ethanone
N⁵-(2-Methoxyethyl)-N⁵-methyl-4-(1H-pyrazol-4-yl)-N²-(pyrimidin-2-yl)thiazole-2,5-diamine
N-(5-Fluoro-4-methylpyrimidin-2-yl)-4-(1H-pyrazol-4-yl)thiazol-2-amine
N-(5-Fluoro-4-methylpyrimidin-2-yl)-5-methyl-4-(1H-pyrazol-4-yl)thiazol-2-amine
4-(3-(Ethyl(methyl)amino)-1H-pyrazol-4-yl)-N-(pyrimidin-2-yl)thiazol-2-amine
4-(3-(Methylamino)-1H-pyrazol-4-yl)-N-(pyrimidin-2-yl)thiazol-2-amine
4-(5-(4-Fluorophenyl)-1H-pyrazol-4-yl)-N-(pyrimidin-2-yl)thiazol-2-amine
4-(5-(Methoxymethyl)-1H-pyrazol-4-yl)-N-(pyrimidin-2-yl)thiazol-2-amine
5-Cyclobutyl-4-(1H-pyrazol-4-yl)-N-(pyrimidin-2-yl)thiazol-2-amine
N-(Pyrimidin-2-yl)-4-(3-(pyrrolidin-1-yl)-1H-pyrazol-4-yl)thiazol-2-amine
4-(3-((2-Methoxyethyl)(methyl)amino)-1H-pyrazol-4-yl)-N-(pyrimidin-2-yl)thiazol-2-amine
N-(4-Methylpyrimidin-2-yl)-4-(3-(pyrrolidin-1-yl)-1H-pyrazol-4-yl)thiazol-2-amine
N-(4-Methylpyrimidin-2-yl)-4-(3-phenyl-1H-pyrazol-4-yl)thiazol-2-amine
(4-(2-(4-Methylpyrimidin-2-ylamino)thiazol-4-yl)-1H-pyrazol-3-yl)methanol
4-(3-(Methoxymethyl)-1H-pyrazol-4-yl)-N-(4-methylpyrimidin-2-yl)thiazol-2-amine
4-(3-(Ethylamino)-1H-pyrazol-4-yl)-N-(4-methylpyrimidin-2-yl)thiazol-2-amine and
4-(3-(2-Methoxyethyl)-1H-pyrazol-4-yl)-N-(4-methylpyrimidin-2-yl)thiazol-2-amine.

4. A compound according to claim 3 having the Formula (I) wherein:
M is an optionally substituted pyrimidinyl;
A is H;
B radical is selected from the group of phenyl and pyridinyl which can be optionally substituted by hydrogen, halogen, —CN, —OCF₃, —(C₁-C₆)alkyl, —(C₃-C₂)cycloalkyl, —(C₀-C₆)alkyl-O—(C₀-C₆)alkyl, —(C₁-C₆)haloalkylene-O—(C₀-C₆)alkyl, —(C₀-C₆)alkyl-N—((C₀-C₆)alkyl)₂, —(C₀-C₆)alkyl-C(═O)—N((C₀-C₆)alkyl)₂, heterocycle and —(C₁-C₆)alkylene-heterocycle;
provided that according to proviso (v) the compound is not:
4-(5-(4-Fluorophenyl)-1H-pyrazol-4-yl)-N-(pyrimidin-2-yl)thiazol-2-amine and
N-(4-Methylpyrimidin-2-yl)-4-(3-phenyl-1H-pyrazol-4-yl)thiazol-2-amine.

5. A compound according to claim 2 having the Formula (I) wherein:
M is an optionally substituted radical selected from the group of oxadiazolyl, oxazolyl, thiadiazolyl and triazinyl.

6. A compound according to claims 1 to 5, which can exist as optical isomers, wherein said compound is either the racemic mixture or one or both of the individual optical isomers.

7. A compound according to claims 1 to 6, wherein said compound is selected from:
4-(3-(Difluoromethyl)-1H-pyrazol-4-yl)-N-(4-methylpyrimidin-2-yl)thiazol-2-amine
N-(4-Methylpyrimidin-2-yl)-4-(3-(3-(1,1-dioxido-4-thiomorpholinyl)phenyl)-1H-pyrazol-4-yl)thiazol-2-amine
4-(3-(2-Fluorophenyl)-1H-pyrazol-4-yl)-N-(4-methylpyrimidin-2-yl)thiazol-2-amine
3-(4-(2-(4-Methylpyrimidin-2-ylamino)thiazol-4-yl)-1H-pyrazol-3-yl)benzonitrile
4-(3-(5-Fluoro-2-methoxyphenyl)-1H-pyrazol-4-yl)-N-(4-methylpyrimidin-2-yl)thiazol-2-amine
2,2,2-Trifluoro-1-(4-(4-(2-(4-methylpyrimidin-2-ylamino)thiazol-4-yl)-1H-pyrazol-3-yl)phenyl)ethanol
4-(4-(2-(4-Methylpyrimidin-2-ylamino)thiazol-4-yl)-1H-pyrazol-3-yl)isoindolin-1-one
4-(3-(5,6-Dihydro-4H-pyrrolo[1,2-b]pyrazol-3-yl)-1H-pyrazol-4-yl)-N-(4-methylpyrimidin-2-yl)thiazol-2-amine
N-(4-Methylpyrimidin-2-yl)-4-(3-(3-(morpholinomethyl)phenyl)-1H-pyrazol-4-yl)thiazol-2-amine
4-(3-(5-Chloropyridin-3-yl)-1H-pyrazol-4-yl)-N-(4-methylpyrimidin-2-yl)thiazol-2-amine
4-(3-(2-Methoxyphenyl)-1H-pyrazol-4-yl)-N-(4-methylpyrimidin-2-yl)thiazol-2-amine
N-(4-Methylpyrimidin-2-yl)-4-(3-m-tolyl-1H-pyrazol-4-yl)thiazol-2-amine
4-(3-Morpholino-1H-pyrazol-4-yl)-N-(pyridin-2-yl)thiazol-2-amine
2-(5-(4-(2-(4-Methylpyrimidin-2-ylamino)thiazol-4-yl)-1H-pyrazol-3-yl)pyridin-3-yl)propan-2-ol
3-(3-(4-(2-(4-Methylpyrimidin-2-ylamino)thiazol-4-yl)-1H-pyrazol-3-yl)phenyl)propan-1-ol
4-(3-(4-Chlorophenyl)-1H-pyrazol-4-yl)-N-(4-methylpyrimidin-2-yl)thiazol-2-amine
4-(3-(4-Cyclopropylphenyl)-1H-pyrazol-4-yl)-N-(4-methylpyrimidin-2-yl)thiazol-2-amine
N-(4-Methylpyrimidin-2-yl)-4-(3-(pyridin-4-yl)-1H-pyrazol-4-yl)thiazol-2-amine
N-(4-Methylpyrimidin-2-yl)-4-(3-o-tolyl-1H-pyrazol-4-yl)thiazol-2-amine
N-(4-Methylpyrimidin-2-yl)-4-(3-(2-(trifluoromethoxy)phenyl)-1H-pyrazol-4-yl)thiazol-2-amine
4-(3-(2-Chlorophenyl)-1H-pyrazol-4-yl)-N-(4-methylpyrimidin-2-yl)thiazol-2-amine
3-(4-(2-(4-Methylpyrimidin-2-ylamino)thiazol-4-yl)-1H-pyrazol-3-yl)benzamide
4-(3-(2-Ethoxyphenyl)-1H-pyrazol-4-yl)-N-(4-methylpyrimidin-2-yl)thiazol-2-amine
4-(3-(2,4-Difluorophenyl)-1H-pyrazol-4-yl)-N-(4-methylpyrimidin-2-yl)thiazol-2-amine
(4-(2-(4-Methylpyrimidin-2-ylamino)thiazol-4-yl)-1H-pyrazol-3-yl)(phenyl)methanone
(4-(2-(4-Methylpyrimidin-2-ylamino)thiazol-4-yl)-1H-pyrazol-3-yl)(phenyl)methanol
Cyclopropyl(4-(2-(4-methylpyrimidin-2-ylamino)thiazol-4-yl)-1H-pyrazol-5-yl)methanone
4-(5-(Cyclobutoxymethyl)-1H-pyrazol-4-yl)-N-(4-methylpyrimidin-2-yl)thiazol-2-amine
4-(5-(1-Methoxyethyl)-1H-pyrazol-4-yl)-N-(4-methylpyrimidin-2-yl)thiazol-2-amine
4-(3-(3-(Dimethylamino)phenyl)-1H-pyrazol-4-yl)-N-(4-methylpyrimidin-2-yl)thiazol-2-amine
4-(3-(5-Fluoro-2-methoxyphenyl)-1H-pyrazol-4-yl)-N-(pyrimidin-2-yl)thiazol-2-amine
N-Methyl-4-(2-(4-methylpyrimidin-2-ylamino)thiazol-4-yl)-1H-pyrazole-5-carboxamide
1-(3-(4-(2-(4-Methylpyrimidin-2-ylamino)thiazol-4-yl)-1H-pyrazol-3-yl)-5,6-dihydropyridin-1(2H)-yl)ethanone
4-Fluoro-3-(4-(2-(4-methylpyrimidin-2-ylamino)thiazol-4-yl)-1H-pyrazol-5-yl)benzonitrile
2-Fluoro-3-(4-(2-(4-methylpyrimidin-2-ylamino)thiazol-4-yl)-1H-pyrazol-5-yl)benzonitrile
N-(3-Fluoro-6-methylpyridin-2-yl)-4-(3-(methoxymethyl)-1H-pyrazol-4-yl)thiazol-2-amine
N-(5-Fluoropyrimidin-2-yl)-4-(3-(methoxymethyl)-1H-pyrazol-4-yl)thiazol-2-amine
N-(4-Ethylpyrimidin-2-yl)-4-(3-(methoxymethyl)-1H-pyrazol-4-yl)thiazol-2-amine
Methyl 3-(4-(2-(4-methylpyrimidin-2-ylamino)thiazol-4-yl)-1H-pyrazol-3-yl)-2,5-dihydro-1H-pyrrole-1-carboxylate
1-(4-(4-(2-(4-Methylpyrimidin-2-ylamino)thiazol-4-yl)-1H-pyrazol-3-yl)-5,6-dihydropyridin-1(2H)-yl)ethanone
Methyl 4-(4-(2-(4-methylpyrimidin-2-ylamino)thiazol-4-yl)-1H-pyrazol-3-yl)-5,6-dihydropyridine-1(2H)-carboxylate
1-(3-(4-(2-(4-Methylpyrimidin-2-ylamino)thiazol-4-yl)-1H-pyrazol-3-yl)phenyl)pyrrolidin-3-ol
3-(4-(2-(Pyrimidin-2-ylamino)thiazol-4-yl)-1H-pyrazol-3-yl)benzonitrile
2-(5-Methyl-1,2,4-thiadiazol-3-ylamino)-4-(1H-pyrazol-4-yl)thiazole-5-carbonitrile
2-(1,2,4-Triazin-3-ylamino)-4-(1H-pyrazol-4-yl)thiazole-5-carbonitrile
1-(2-(4-Methylpyrimidin-2-ylamino)-4-(1H-pyrazol-4-yl)thiazol-5-yl)ethanone
and a pharmaceutically acceptable acid or base addition salt thereof, a stereochemically isomeric form thereof and an N-oxide form thereof.

8. A compound according to claims 1 to 6, wherein said compound is selected from:
5-Ethoxy-N-(4-methylpyrimidin-2-yl)-4-(1H-pyrazol-4-yl)thiazol-2-amine
and a pharmaceutically acceptable acid or base addition salt thereof, a stereochemically isomeric form thereof and an N-oxide form thereof.

9. A pharmaceutical composition comprising a therapeutically effective amount of a compound according to claims 1 to 8 and a pharmaceutically acceptable carrier and/or excipient.

10. A method of treating or preventing a condition in a mammal, including a human, the treatment or prevention of which is affected or facilitated by the neuromodulatory effect of mGluR4 allosteric modulators, comprising administering to a mammal in need of such treatment or prevention, an effective amount of a compound/composition according to claims 1 to 9.

11. A method of treating or preventing a condition in a mammal, including a human, the treatment or prevention of which is affected or facilitated by the neuromodulatory effect of mGluR4 positive allosteric modulators, comprising administering to a mammal in need of such treatment or prevention, an effective amount of a compound/composition according to claims 1 to 9.

12. A method useful for treating or preventing central nervous system disorders selected from the group consisting of: addiction, tolerance or dependence; affective disorders, such as depression and anxiety; psychiatric disease such as psychotic disorders, attention-deficit/hyperactivity disorder and bipolar disorder; Parkinson's disease, memory impairment, Alzheimer's disease, dementia, delirium tremens, other forms of neurodegeneration, neurotoxicity, and ischemia, comprising administering to a mammalian patient in need of such treatment or prevention, an effective amount of a compound/composition according to claims 1 to 9.

13. A method useful for treating or preventing central nervous system disorders selected from the group consisting of: Parkinson's disease and movement disorders such as bradykinesia, rigidity, dystonia, drug-induced parkinsonism, dyskinesia, tardive dyskinesia, L-DOPA-induced dyskinesia, dopamine agonist-induced dyskinesia, hyperkinetic movement disorders, Gilles de la Tourette syndrome, resting tremor, action tremor, akinesia, akinetic-rigid syndrome, akathisia, athetosis, asterixis, tics, postural instability, postencephalitic parkinsonism, muscle rigidity, chorea and choreaform movements, spasticity, myoclonus, hemiballismus, progressive supranuclear palsy, restless legs syndrome, and periodic limb movement disorder, comprising administering to a mammalian patient in need of such treatment or prevention, an effective amount of a compound/composition according to claims 1 to 9.

14. A method of claim 13 comprising administering to a mammalian patient in need of such treatment or prevention, an effective amount of a compound/composition according to claims 1 to 9 in combination with an agent selected from the group consisting of: levodopa, levodopa with a selective extracerebral decarboxylase inhibitor, carbidopa, entacapone, a COMT inhibitor, a dopamine agonist, an anticholinergic, a cholinergic agonist, a butyrophenone neuroleptic agent, a diphenylbutylpiperidine neuroleptic agent, a heterocyclic dibenzazepine neuroleptic agent, an indolone neuroleptic agent, a phenothiazine neuroleptic agent, a thioxanthene neuroleptic agent, an NMDA receptor antagonist, an MAO-B inhibitor, an mGluR5 antagonist or an A_{2A} antagonist.

15. A method useful for treating or preventing central nervous system disorders selected from the group consisting of: cognitive disorders such as delirium, substance-induced persisting delirium, dementia, dementia due to HIV disease, dementia due to Huntington's disease, dementia due to Parkinson's disease, Parkinsonian-ALS demential complex, dementia of the Alzheimer's type, substance-induced persisting dementia, and mild cognitive impairment, comprising administering to a mammalian patient in need of such treatment or prevention, an effective amount of a compound/composition according to claims 1 to 9.

16. A method useful for treating affective disorders selected from the group consisting of: anxiety, agoraphobia, generalized anxiety disorder (GAD), obsessive-compulsive disorder (OCD), panic disorder, post-traumatic stress disorder (PTSD), social phobia, other phobias, substance-induced anxiety disorder, and acute stress disorder, comprising administering to a mammalian patient in need of such treatment, an effective amount of a compound/composition according to claims 1 to 9.

17. A method useful for treating or preventing central nervous system disorders selected from the group consisting of: mood disorders, bipolar disorders (I & II), cyclothymic disorder, depression, dysthymic disorder, major depressive disorder, and substance-induced mood disorder, comprising administering to a mammalian patient in need of such treatment or prevention, an effective amount of a compound/composition according to claims 1 to 9.

18. A method useful for treating or preventing neurological disorders selected from the group consisting of neurodegeneration, neurotoxicity or ischemia such as stroke, spinal cord injury, cerebral hypoxia, intracranial hematoma, Parkinson's disease, memory impairment, Alzheimer's disease, dementia, and delirium tremens, comprising administering to a mammalian patient in need of such treatment or prevention, an effective amount of a compound/composition according to claims 1 to 9.

19. A method useful for treating or preventing inflammatory central nervous system disorders selected from the group consisting of: multiple sclerosis forms such as benign multiple sclerosis, relapsing-remitting multiple sclerosis, secondary progressive multiple sclerosis, primary progressive multiple sclerosis, and progressive-relapsing multiple sclerosis, comprising administering to a mammalian patient in need of such treatment or prevention, an effective amount of a compound/composition according to claims 1 to 9.

20. A method useful for treating or preventing migraine, comprising administering to a mammalian patient in need of such treatment or prevention, an effective amount of a compound/composition according to claims 1 to 9.

21. A method useful for treating or preventing epilepsy and tremor, temporal lobe epilepsy, epilepsy secondary to another disease or injury such as chronic encephalitis, traumatic brain injury, stroke or ischemia, comprising administering to a mammalian patient in need of such treatment or prevention, an effective amount of a compound/composition according to claims 1 to 9.

22. A method useful for treating or preventing inflammation and/or neurodegeneration resulting from traumatic brain injury, stroke, ischemia, spinal cord injury, cerebral hypoxia or intracranial hematoma, comprising administering to a mammalian patient in need of such treatment or prevention, an effective amount of a compound/composition according to claims 1 to 9.

23. A method useful for treating or preventing sensory, motor or cognitive symptoms resulting from traumatic brain injury, stroke, ischemia, spinal cord injury, cerebral hypoxia or intracranial hematoma, comprising administering to a mammalian patient in need of such treatment or prevention, an effective amount of a compound/composition according to claims 1 to 9.

24. A method useful for treating medulloblastomas, comprising administering to a mammalian patient in need of such treatment, an effective amount of a compound/composition according to claims 1 to 9.

25. A method useful for treating or preventing inflammatory or neuropathic pain, comprising administering to a mammalian patient in need of such treatment or prevention, an effective amount of a compound/composition according to claims 1 to 9.

26. A method useful for treating, preventing, ameliorating, controlling or reducing the risk of various metabolic disorders associated with glutamate dysfunction, comprising administering to a mammalian patient in need of such treatment, prevention, amelioration, control or reduction of risk, an effective amount of a compound/composition according to claims 1 to 9.

27. A method useful for treating or preventing type 2 diabetes, comprising administering to a mammalian patient in need of such treatment or prevention, an effective amount of a compound/composition according to claims 1 to 9.

28. A method useful for treating or preventing diseases or disorders of the retina, retinal degeneration or macular degeneration, comprising administering to a mammalian patient in need of such treatment or prevention, an effective amount of a compound/composition according to claims 1 to 9.

29. A method useful for treating or preventing diseases or disorders of the gastrointestinal tract including gastroesophageal reflux disease (GERD), lower esophageal sphincter diseases or disorders, diseases of gastrointestinal motility, colitis, Crohn's disease or irritable bowel syndrome (IBS), comprising administering to a mammalian patient in need of such treatment or prevention, an effective amount of a compound/composition according to claims 1 to 9.

30. Use of a compound according to claims 1 to 8 in the manufacture of a medicament for a use as defined in any of claims 10 to 29.

31. Use of a compound according to claims 1 to 8 to prepare a tracer for imaging a metabotropic glutamate receptor.

32. Use of a compound according to claims 1 to 8 as a taste agent, flavour agent, flavour enhancing agent or a food or beverage additive.

33. A compound according to claims 1 to 8 or a composition according to claim 9 for a use in a treatment or prevention as defined in any of claims 10 to 15, 17 to 23, 25 and 27 to 29.

34. A compound according to claims 1 to 8 or a composition according to claim 9 for a use as defined in claim 26.

35. A compound according to claims 1 to 8 or a composition according to claim 9 for a use in a treatment as defined in any of claims 16 and 24.
